(19) United States
(12) Patent Application Publication
(10) Pub. No.: US 2025/0283123 A1
(43) Pub. Date: Sep. 11, 2025

(54) ETHANE OR ETHANOL INTO 3-HYDROXYPROPIONATE USING AN ENGINEERED MICROORGANISM

(71) Applicant: INDUSTRIAL MICROBES, INC., Alameda, CA (US)

(72) Inventors: Elizabeth Jane CLARKE, Alameda, CA (US); Noah Charles HELMAN, Alameda, CA (US); Timothy Brian ROTH, Alameda, CA (US); Yuan ZHANG, Alameda, CA (US)

(73) Assignee: INDUSTRIAL MICROBES, INC., Alameda, CA (US)

(21) Appl. No.: 18/272,499

(22) PCT Filed: Jan. 28, 2022
(86) PCT No.: PCT/US2022/014288

§ 371 Date: Jul. 14, 2023

Related U.S. Application Data
(60) Provisional application No. 63/143,553, filed on Jan. 29, 2021.

(51) Int. Cl. C12P 7/625; C12N 1/20; C12N 9/00; C12N 9/02; C12N 15/70; C12P 7/42; C12R 1/19

(52) U.S. Cl. CPC C12P 7/625; C12N 1/20; C12N 9/0073; C12N 9/93; C12N 15/70; C12P 7/42; C12R 2001/19; C12Y 114/13025; C12Y 604/01002

(57) ABSTRACT

Provided are synthetic organisms and methods for the conversion of ethane and related substrates into 3-hydroxypropionate and related products.

### CROSS REFERENCE TO RELATED APPLICATIONS

This application is a 35 U.S.C. 371 National Stage of International Application Number PCT/US2022/014288, Filed Jan. 28, 2022 which claims priority to and the benefit of U.S. provisional patent application No. 63/143,553, filed on Jan. 29, 2021, the entirety of which is hereby incorporated by reference.

### GOVERNMENT INTEREST

This invention was made in part with government support under Grant No. DE-SC0018831 awarded by the Department of Energy. The government has certain rights in the invention.

### SEQUENCE LISTING

The instant application contains a Sequence Listing which has been submitted electronically in ASCII format and is hereby incorporated by reference in its entirety. Said ASCII copy, created on Jan. 26, 2022, is named 1107367_000040 SL.txt and is 148,177 bytes in size.

Provided are synthetic organisms and methods for the conversion of ethane and related substrates into 3-hydroxypropionate and related products.

### BACKGROUND

Microorganisms can be engineered to manipulate the use of raw materials or substrates required and/or the range of products formed. An engineered microorganism can often provide products at a low cost and with a low environmental impact.

Sugars, such as simple sugars, disaccharides, starches, carbohydrates, cellulosic sugars, and sugar alcohols, are often used as a raw material for engineered microorganisms. But, sugar can be expensive. Microorganisms engineered to use raw materials or substrates other than sugar would be useful.

For example, two-carbon compounds, such as ethane, could be used as a raw material for engineered microorganisms. Ethane is plentiful and is significantly less expensive than sugar. And, ethane is expected to remain inexpensive for decades to come. Microorganisms engineered to use ethane rather than sugar would be extremely useful.

As a product, it would be extremely advantageous to be able to make 3-hydroxypropionate (and 3-hydroxypropionic acid) with engineered microorganisms. 3-hydroxypropionate can be easily converted to a range of bulk chemicals, such as acrylic acid, ethyl acrylate, butyl acrylate, 1,3-propanediol (1,3-PD), 3-hydroxypropionaldehyde (3-HPA), and/or malonic acid. Such bulk chemicals can be used to make high-performance plastics, water-soluble paints, coatings, fibers, adhesives, chemicals for industrial water treatment, and super-absorbent polymers for diapers.

It would be useful to have synthetic organisms capable of using an inexpensive substrate, such as ethane, to make a valuable product, such as 3-hydroxypropionate.

### BRIEF DESCRIPTION

The invention is drawn to synthetic microorganisms comprising exogenous polynucleotides that improve the production of a product from a substrate. The substrate may be ethane and the product may be 3-hydroxypropionate or a related product. The exogenous polynucleotide may encode an ethane monooxygenase, an ethylotrophy pathway, and/or a malonyl-CoA-product pathway. Surprisingly, integration of the exogenous polynucleotide substantially improves production of a product in synthetic microorganisms.

A first aspect provides a synthetic microorganism comprising or consisting of at least one exogenous polynucleotide that encodes one or more polypeptides comprising or consisting of an ethane monooxygenase, an ethylotrophy pathway, and/or a malonyl-CoA-product pathway. The at least one exogenous polynucleotide may be inserted or integrated into the genome of the microorganism.

The ethane monooxygenase comprises or consists of an ethane monooxygenase having an amino acid sequence that is more than about 70%, more than about 75%, more than about 80%, more than about 85%, more than about 90%, or more than about 95% identical or identical to any of SEQ ID Nos: 1-16. The ethylotrophy pathway comprises or consists of one or more enzymes having an amino acid sequence that is more than about 70%, more than about 75%, more than about 80%, more than about 85%, more than about 90%, or more than about 95% identical or identical to any of SEQ ID Nos: 22-23. The malonyl-CoA-product pathway comprises or consists of one or more enzymes having an amino acid sequence that is more than about 70%, more than about 75%, more than about 80%, more than about 85%, more than about 90%, or more than about 95% identical or identical to any of SEQ ID Nos: 29-30.

The synthetic microorganism generates a product comprising or consisting of 3-hydroxypropionic acid. The synthetic microorganism may generate a product comprising or consisting of a polymer of 3-hydroxypropionic acid. The synthetic microorganism may generate one or more products comprising or consisting of fatty acids or fatty acid derivatives. The synthetic microorganism may generate a product comprising or consisting of polymer(s) of 3-hydroxy-fatty acids. Finally, the synthetic microorganism may generate a product comprising or consisting of malonic acid.

The synthetic microorganism may further comprise one or more exogenous polynucleotides which overexpress acetyl-CoA carboxylase. The synthetic microorganism may be derived from any microorganism. In some embodiments, the synthetic microorganism is derived from Escherichia coli.

A second aspect provides a method of producing a product, the method comprising or consisting of culturing a synthetic microorganism comprising or consisting of at least one exogenous polynucleotide that encodes one or more polypeptides comprising or consisting of an ethane monooxygenase, an ethylotrophy pathway, and/or a malonyl-CoA-product pathway for a suitable period of time and under conditions sufficient to lead to improved production of the product. The at least one exogenous polynucleotide may be inserted or integrated into the genome of the microorganism.

The ethane monooxygenase comprises or consists of an ethane monooxygenase having an amino acid sequence that is more than about 70%, more than about 75%, more than about 80%, more than about 85%, more than about 90%, or more than about 95% identical or identical to any of SEQ ID Nos: 1-16. The ethylotrophy pathway comprises or consists of one or more enzymes having an amino acid sequence that is more than about 70%, more than about 75%, more than about 80%, more than about 85%, more than about 90%, or more than about 95% identical or identical to any of SEQ ID Nos: 22-23. The malonyl-CoA-product pathway comprises or consists of one or more enzymes having an amino acid sequence that is more than about 70%, more than about 75%, more than about 80%, more than about 85%, more than about 90%, or more than about 95% identical or identical to any of SEQ ID Nos: 29-30.

The synthetic microorganism generates a product comprising or consisting of 3-hydroxypropionic acid. The synthetic microorganism may generate a product comprising or consisting of a polymer of 3-hydroxypropionic acid. The synthetic microorganism may generate one or more products comprising or consisting of fatty acids or fatty acid derivatives. The synthetic microorganism may generate a product comprising or consisting of polymer(s) of 3-hydroxy-fatty acids. Finally, the synthetic microorganism may generate a product comprising or consisting of malonic acid.

The synthetic microorganism may further comprise one or more exogenous polynucleotides which overexpress acetyl-CoA carboxylase. The synthetic microorganism may be derived from any derived from any microorganism. In some embodiments, the synthetic microorganism is derived from Escherichia coli.

The synthetic microorganism is cultured for a suitable period of time and under conditions sufficient to lead to improved production of the product.

A third aspect provides a nucleic acid suitable for integration into a synthetic microorganism, wherein the nucleic acid comprises or consists of at least one polynucleotide that encodes one or more polypeptides comprising or consisting of an ethane monooxygenase, an ethylotrophy pathway, and/or a malonyl-CoA-product pathway. The at least one polynucleotide may be inserted or integrated into the genome of a microorganism.

The ethane monooxygenase comprises or consists of an ethane monooxygenase having an amino acid sequence that is more than about 70%, more than about 75%, more than about 80%, more than about 85%, more than about 90%, or more than about 95% identical or identical to any of SEQ ID Nos: 1-16. The ethylotrophy pathway comprises or consists of one or more enzymes having an amino acid sequence that is more than about 70%, more than about 75%, more than about 80%, more than about 85%, more than about 90%, or more than about 95% identical or identical to any of SEQ ID Nos: 22-23. The malonyl-CoA-product pathway comprises or consists of one or more enzymes having an amino acid sequence that is more than about 70%, more than about 75%, more than about 80%, more than about 85%, more than about 90%, or more than about 95% identical or identical to any of SEQ ID Nos: 29-30.

Any suitable microorganism may be used with the nucleic acid. In some embodiments, the synthetic microorganism is derived from Escherichia coli.

### BRIEF DESCRIPTION OF THE DRAWINGS

“FIG. 1A-C depicts results for experiments in which a culture was split into two vials after which the headspace was injected with either ethane or nitrogen. FIG. 1A depicts the total amount of 3HP produced for cultures of LC805, LC807, and LC809 with starting OD of ˜1.6, 1.4, and 2.3, respectively. FIG. 1B depicts the same data normalized by starting OD. FIG. 1C depicts a repeat experiment with LC 805 and LC809 at two starting ODs.”

FIG. 2 depicts results for an experiment with LC805 and LC809 in which cultures were split into two vials after which the headspace was injected with either ethane or nitrogen. Additionally, duplicate cultures, one with 0.5% ethanol in the seed train and one without ethanol were used. The total amount of 3HP produced by cultures was determined.

FIG. 3 shows a plasmid comparison of the plasmids from the invention.

### DETAILED DESCRIPTION

### A. Definitions

The invention is drawn to synthetic microorganisms comprising exogenous polynucleotides that improve the production of a product from a substrate. The substrate may ethane and the product may be 3-hydroxypropionate or a related product. The exogenous polynucleotide may encode an ethane monooxygenase, an ethylotrophy pathway, and/or a malonyl-CoA-product pathway. Surprisingly, integration of the exogenous polynucleotide substantially improves production of a product in synthetic microorganisms.

As used herein, “amino acid” shall mean those organic compounds containing amine (—NH₂) and carboxyl (—COOH) functional groups, along with a side chain (R group) specific to each amino acid. The key elements of an amino acid are carbon (C), hydrogen (H), oxygen (O), and nitrogen (N), although other elements are found in the side chains of certain amino acids.

As used herein, the terms “chaperone,” “protein folding chaperone” and “folding chaperone” are intended to mean one or more proteins that improve the folding of polypeptide (amino acid) chains into 3-dimensional structures. Protein folding chaperones help their substrates, namely other proteins, to become properly folded and often more highly soluble. Since most proteins must be folded in a particular shape to be functional, the expression of protein folding chaperones can assist in the proper assembly of certain enzymes in a cell and thereby can result in an increase in the enzymatic activity of the substrate proteins.

As used herein, “conservative amino acid substitution(s)” or “conservative substitution(s)” refers to a substitution in which an amino acid residue is substituted by another amino acid residue having a side chain (R group) with similar chemical properties (e.g., charge or hydrophobicity). In general, a conservative amino acid substitution should not substantially change the functional properties of a protein. The following six groups each contain amino acids that are often, depending upon context, considered conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Alanine (A), Valine (V), and 6) Phenylalanine (F), Tyrosine (Y), Tryptophan (W).

As used herein, the term “culturing” is intended to mean the growth or maintenance of a microorganism under laboratory or industrial conditions. The culturing of microorganisms is a standard practice in the field of microbiology. Microorganisms can be cultured using liquid or solid media as a source of nutrients for the microorganisms. In addition, some microorganisms can be cultured in defined media, in which the liquid or solid media are generated by preparation using purified chemical components. The composition of the culture media can be adjusted to suit the microorganism or the industrial purpose for the culture.

As used herein, the term “dehydrogenase” is intended to mean an enzyme belonging to the group of oxidoreductases that oxidizes a substrate by a reduction reaction that removes one or more hydrogen atoms from a substrate to an electron acceptor.

As used herein, the term “enzyme” or “enzymatically” shall refer to biological catalysts. Enzymes accelerate, or catalyze, chemical reactions. Like all catalysts, enzymes increase the rate of reaction by lowering the activation energy.

As used herein, “exogenous” is intended to mean something, such as a gene or polynucleotide, that originates outside of the organism of concern or study. An exogenous polynucleotide, for example, may be introduced into an organism by introduction into the organism of an encoding nucleic acid, such as, for example, by integration into a host chromosome or by introduction of a plasmid. When used in reference to a biosynthetic activity, the term refers to an activity that is introduced into a reference organism, such as a microorganism or synthetic culture as set forth in the invention. As an example, exogenous expression of an encoding nucleic acid can utilize either or both a heterologous or homologous encoding nucleic acid. A nucleic acid need not include all of its relevant or even complete coding regions on a single polymer and the invention provided herein contemplates having complete or partial coding sequences on different polymers.

As used herein, the term “exogenous polynucleotides” is intended to mean polynucleotides that are not derived from naturally occurring polynucleotides in a given organism. Exogenous polynucleotides may be derived from polynucleotides present in a different organism. The exogenous polynucleotides can be introduced into the organism by introduction of an encoding nucleic acid into the host genetic material such as by integration into a host chromosome or as non-chromosomal (episomal) genetic material such as a plasmid. Therefore, the term as it is used in reference to expression of an encoding nucleic acid refers to introduction of the encoding nucleic acid in an expressible form into the microbial organism. When used in reference to a biosynthetic activity, the term refers to an activity that is introduced into the host reference organism. The source can be, for example, a homologous or heterologous encoding nucleic acid that expresses the referenced activity following introduction into the host microbial organism.

The term “heterologous” refers to a molecule or activity derived from a source other than the referenced species. As set forth in the invention a nucleic acid need not include all of its relevant or even complete coding regions on a single polymer and the invention provided herein contemplates having complete or partial coding regions on different polymers.

As used herein, “integrated,” as used in reference to a polynucleotide, refers to an exogenous polynucleotide that has been inserted in a chromosome of an organism, especially a polynucleotide comprising a coding sequence that can be expressed by the organism. In this respect, phrases such as “integrated into the genome of the synthetic microorganism” “integrated into the host genome,” “integrated into a host chromosome,” and the like have equivalent meanings.

As used herein, a “gene” is a sequence of DNA or RNA which codes for a molecule that has a function. The DNA is first copied into RNA. The RNA can be directly functional or be the intermediate template for a protein that performs a function.

As used herein, “modification,” “genetic alteration,” “genetically altered,” “genetic engineering,” “genetically engineered,” “genetic modification,” “genetically modified,” “genetic regulation,” or “genetically regulated” shall be used interchangeably and refer to direct or indirect manipulation of an organism's genome or genes to produce, for example, a desired effect, such as a desired phenotype. Genetic alteration includes a set of technologies that can be used to change genetic makeup, which ultimately could lead to the suppression or enhancement of phenotype or expression of a gene, as used herein. Genetic alteration shall also include the ability to reduce or prevent expression of a gene or genes. Genetic alteration techniques shall include, for example, but are not be limited to, molecular cloning, gene knockouts, gene targeting, mutation, homologous recombination, gene deletion, gene knockdown, gene silencing, gene addition, genome editing, gene attenuation, or any technique that may be used to suppress or alter the expression of a gene and a phenotype as known to one skilled in the art. Gene modification may be performed by any method known to one skilled in the art such as, for example, without limitation, CRISPR.

As used herein, “gene deletion” or “deletion” refers to a mutation or genetic modification in which a sequence of DNA is lost, deleted, or modified. A gene may be deleted to alter an organism's genome or to produce a desired effect or desired phenotype. Gene deletion may be used, for example, without limitation, as a method to suppress, alter, or enhance a particular phenotype.

As used herein, the term “gene knockdown” refers to a technique by which expression of one or more genes are reduced. Reduction can occur by any method known to one skilled in the art such as genetic modification, CRISPR interference, or by treatment with a reagent such as a short DNA or RNA oligonucleotide that has a sequence complimentary to either a gene or an mRNA transcript.

As used herein, the term “gene knockout” refers to a procedure whereby a gene is made inoperative.

As used herein, “gene silencing,” “silencing,” or “silenced” refers to the regulation of a gene, in particular, without limitation, the down regulation of a gene. Specifically, the term refers to the ability to reduce or prevent the expression of a certain gene. Gene silencing can occur at any cellular process, such as, for example, without limitation, during transcription or translation. Any methods of gene silencing well known in the art may be used such as, for example, without limitation, RNA interference and the use of antisense oligonucleotides.

As used herein, the term “homology” or “homologous” refer to the degree of biological shared ancestry in the evolutionary history of life. Homology or homologous may also refer to sequence homology, the biological homology between protein or polynucleotide sequences with respect to shared ancestry as determined by the closeness of nucleotide or protein sequences. Homology among proteins or polynucleotides is typically inferred from their sequence similarity. Alignments of multiple sequences are used to indicate which regions of each sequence are homologous. The term “percent homology” often refers to “sequence similarity.” The percentage of identical residues (percent identity) or the percentage of residues conserved with similar physiochemical properties (percent similarity), e.g. leucine and isoleucine, is usually used to quantify homology. Partial homology can occur where a segment of the compared sequences has a shared origin.

As used herein, the term “identity” refers to a quantitative measurement of the similarity between two sequences (DNA, amino acid, or otherwise).

As used herein, the term “improved production of a product from a substrate” is intended to mean a situation in which a synthetic microorganism or synthetic culture has been modified in some way, such as, for example, without limitation, through genetic modification or integration, so that, under a set of conditions and relative to the original strain, the modified strain produces a product from the substrate or produces a product from the substrate faster than the rate or amount, respectively, from an unmodified microorganism or synthetic culture. A direct comparison of two strains can be made by growing the microorganisms or synthetic cultures under identical conditions and measuring the amount of product produced by each.

As used herein, “ethane monooxygenase” means the class of enzymes and enzyme complexes capable of oxidizing a carbon-hydrogen bond of the ethane molecule to result in a molecule of ethanol. In some embodiments, the ethane monooxygenase comprises or consists of, for example, without limitation, a methane monooxygenase such as, for example, sMMO and/or pMMO, a butane monooxygenase, a propane monooxygenase, an ammonium monooxygenase, p450 (such as, for example, P450 BM3 and/or P450cam), and/or AlkB.

As used herein, “monooxygenase,” “methane monooxygenase enzyme,” “MMO” “MO,” or “soluble diiron monooxygenase” are intended to mean the class of enzymes and enzyme complexes capable of oxidizing a carbon-hydrogen bond of the methane molecule or other short chain alkane to result methanol or another molecule. Naturally occurring methane-consuming microorganisms have evolved at least two classes of monooxygenase enzymes: soluble monooxygenases (“sMMO”) or and particulate monooxygenase (“pMMO”). Any enzyme or enzyme complex of these categories, any mutated enzyme or complex, or any researcher-designed enzyme or enzyme complex that converts methane into methanol would be considered a methane monooxygenase enzyme. Many of these enzymes may also oxidize a wide range of substrates, such as ethane into ethanol, and thus act as an ethane monooxygenase.

As used herein, “ethylotrophy pathway” is intended to mean a metabolic pathway that utilizes reduced carbon substrates containing a single carbon-carbon bond, such as, for example, without limitation, ethane, ethanol, acetaldehyde, acetate, ethylene, and monoethylene glycol.

As used herein, “malonyl-CoA-product pathway” is intended to mean a pathway that uses malonyl-CoA as a reactant or intermediate molecule to produce a product of interest, such as, for example, without limitation, malonic acid, 3-hydroxypropionic acid, fatty acids, 3-hydroxyfatty acids, fatty alcohols, fatty esters, adipic acid, 2-butanone, 3-pentanone, or salts or derivatives thereof (see, for example, https://agilebiofoundry.org/wp-content/uploads/2020/09/ABF-Metabolic-Map-12-for-survey.pdf for products derived from malonyl-CoA as Beachhead Molecule 11, which is incorporated by reference herein).

As used herein, the terms “microbe”, “microbial,” “microbial organism” or “microorganism” are intended to mean any organism that exists as a microscopic cell that is included within the domains of archaea, bacteria, or eukarya. The term is intended to encompass prokaryotic or eukaryotic cells or organisms having a microscopic size and includes bacteria, archaea, and eubacteria of all species as well as eukaryotic microorganisms such as yeast and fungi. The term also includes cell cultures of any species that can be cultured for the production of a product.

As used herein, “naturally occurring” shall refer to microorganisms or cultures normally found in nature.

As used herein, an “operon” shall refer to a functioning unit of genomic DNA containing a cluster of genes under the control of a single promoter. The genes are transcribed together into an mRNA strand and either translated together in the cytoplasm or undergo trans-splicing to create monocistronic mRNAs that are translated separately, i.e. several strands of mRNA that each encode a single gene product. The result of this is that the genes contained in the operon are either expressed together or not at all. Several genes may be co-transcribed to define an operon.

The terms “polynucleotide,” “oligonucleotide,” “nucleotide sequence,” and “nucleic acid sequence” are intended to mean one or more polymers of nucleic acids and include, but are not limited to, coding regions, which are transcribed and translated into a polypeptide such as a chaperone, appropriate regulatory or control sequences, e.g., translational start and stop codons, promoter sequences, ribosome binding sites, polyadenylation signals, transcription factor binding sites, termination sequences, and regulatory domains and enhancers, among others. A polynucleotide, as used herein, need not include all of its relevant or even complete coding regions on a single polymer and the invention provided herein contemplates having complete or partial coding region on different polymers.

As used herein, a “peptide” refers to short chains of amino acid monomers linked by peptide (amide) bonds. Covalent chemical bonds are formed when the carboxyl group of one amino acid reacts with the amino group of another.

As used herein, a “polypeptide” or “protein” is a long, continuous, and unbranched peptide chain. Peptides are normally distinguished from polypeptides and proteins on the basis of size, and as an arbitrary benchmark can be understood to contain approximately 50 or fewer amino acids. Proteins consist of one or more polypeptides arranged in a biologically functional way, often bound to ligands such as coenzymes and cofactors, or to another protein or other macromolecule, such as, for example, DNA or RNA.

Amino acids that have been incorporated into peptides are termed “residues” due to the release of either a hydrogen ion from the amine end or a hydroxyl ion from the carboxyl end, or both, as a water molecule is released during formation of each amide bond. All peptides except cyclic peptides have an N-terminal and C-terminal residue at the ends of the peptide.

As used herein, “product” shall refer to 3-hydroxypropionate and 3-hydroxypropionic acid and related molecules and derivatives. Related molecules include, for example, without limitation, acrylic acid, ethyl acrylate, butyl acrylate, other acrylic acid esters, 1,3-propanediol (1,3-PD), 3-hydroxypropionaldehyde (3-HPA), and malonic acid. Related products also include polymerized forms of 3-hydroxypropionate, polymerized forms of acrylic acid, and polymerized forms of acrylic acid derivatives. Related products further include substances that derived from malonyl-CoA.

As used herein, “promoter” shall refer to a region of DNA that initiates transcription of a particular gene. Promoters are located near the transcription start sites of genes, on the same strand and upstream on the DNA (towards the 5′ region of the sense strand). Promoters can be about 30-1000 base pairs long.

As used herein, the term “substrate” shall refer to a chemical species being used in a chemical reaction. In some embodiments, the substrate is ethane or a related molecule.

As used herein, “sufficient period of time” shall refer to a time period required to grow microorganisms or a synthetic culture to produce a product, such as, for example, a product of interest. In that sense, a sufficient period of time can be the amount of time that enables the microorganisms, or enables the synthetic culture of interest, to produce the product. For example, without limitation, an industrial scale culture may require as little as 5 minutes to begin production of detectable amounts of a product. Some synthetic cultures may be active for weeks.

As used herein, the term “suitable conditions” is intended to mean any set of culturing parameters that provide the microorganism with an environment that enables the culture to consume the available nutrients. In so doing, the microbiological culture may grow and/or produce products, chemicals, or by-products. Culturing parameters may include, but not be limited to, such features as the temperature of the culture media, the dissolved oxygen concentration, the dissolved carbon dioxide concentration, the rate of stirring of the liquid media, the pressure in the vessel, etc.

As used herein, the term “synthetic” is intended to mean a culture or microorganism, for example, without limitation, that has been manipulated into a form not naturally occurring or normally found in nature. For example, a synthetic culture or microorganism shall include, without limitation, a culture or microorganism that has been manipulated to express a polypeptide that is not naturally expressed, or transformed to include a synthetic polynucleotide of interest that is not normally included.

As used herein, the term “synthetic culture” is intended to mean at least one microorganism, or group of microorganisms, that has been manipulated into a form not normally found in nature.

### B. Synthetic Cultures

A first aspect provides a synthetic microorganism comprising or consisting of at least one exogenous polynucleotide that encodes one or more polypeptides comprising or consisting of an ethane monooxygenase, an ethylotrophy pathway, and/or a malonyl-CoA-product pathway. The at least one exogenous polynucleotide may be inserted or integrated into the genome of the microorganism.

The ethane monooxygenase comprises or consists of an ethane monooxygenase having an amino acid sequence that is more than about 70%, more than about 75%, more than about 80%, more than about 85%, more than about 90%, or more than about 95% identical or identical to any of SEQ ID Nos: 1-16. The ethylotrophy pathway comprises or consists of one or more enzymes having an amino acid sequence that is more than about 70%, more than about 75%, more than about 80%, more than about 85%, more than about 90%, or more than about 95% identical or identical to any of SEQ ID Nos: 22-23. The malonyl-CoA-product pathway comprises or consists of one or more enzymes having an amino acid sequence that is more than about 70%, more than about 75%, more than about 80%, more than about 85%, more than about 90%, or more than about 95% identical or identical to any of SEQ ID Nos: 29-30.

The synthetic microorganism generates a product comprising or consisting of 3-hydroxypropionic acid. The synthetic microorganism may generate a product comprising or consisting of a polymer of 3-hydroxypropionic acid. The synthetic microorganism may generate one or more products comprising or consisting of fatty acids or fatty acid derivatives. The synthetic microorganism may generate a product comprising or consisting of polymer(s) of 3-hydroxy-fatty acids. Finally, the synthetic microorganism may generate a product comprising or consisting of malonic acid.

The synthetic microorganism may further comprise one or more exogenous polynucleotides which overexpress acetyl-CoA carboxylase. The synthetic microorganism may be derived from any derived from any microorganism. In some embodiments, the synthetic microorganism is derived from Escherichia coli.

Seed cultures may initially be grown in a rich medium, such as LB. After expansion of the population, the culture is diluted with fresh rich medium containing inducers to turn on expression of ethylotrophy enzymes encoded by exogenous polynucleotides under the control of inducible promoters. This induction medium may also be supplemented with ethanol to induce expression of endogenous enzymes useful for ethylotrophy. The bacterial cells are then separated from the rich medium and resuspended in a minimal medium, for example BEM6, and grown under an ethane containing atmosphere.

In some embodiments, minimal medium is supplemented with ethanol instead of fermenting in the presence of ethane. In such embodiments, the microorganism may not have been modified with an ethane monooxygenase or, if the ethane monooxygenase is under the control of an inducible promoter, the inducer may have been omitted from the induction culture.

### C. Microorganisms

Synthetic microorganisms may be derived from any archaea, bacteria, or eukarya known to one skilled in the art. In some embodiments, the synthetic microorganism is derived from at least one of Escherichia coli, Bacillus subtilis, Bacillus methanolicus, Pseudomonas putida, Saccharomyces cerevisiae, Pichia pastoris, Pichia methanolica, Salmonella enterica, Corynebacterium glutamicum, Klebsiella oxytoca, Anaerobiospirillum succiniciproducens, Actinobacillus succinogenes, Mannheimia succiniciproducens, Rhizobium etli, Gluconobacter oxydans, Zymomonas mobilis, Lactococcus lactis, Lactobacillus plantarum, Streptomyces coelicolor, Clostridium acetobutylicum, Pseudomonas fluorescens, Schizosaccharomyces pombe, Kluyveromyces lactis, Kluyveromyces marxianus, Aspergillus terreus, Aspergillus niger, Yarrowia lipolytica, Hansenula polymorpha, Issatchenkia orientalis, Candida sonorensis, Candida methanosorbosa, and Candida utilis. In some embodiments, the microorganism is derived from Escherichia coli.

In some embodiments, the synthetic organism comprises or consists of one or more microorganisms. In some embodiments, for example, without limitation, conversion of ethane into ethanol is catalyzed in one microorganism and conversion of ethanol into 3HP is catalyzed in a second, genetically distinct microorganism. In some embodiments, conversion of ethane into ethanol and conversion of ethanol into 3HP are both catalyzed in a single microorganism. In some embodiments, a single synthetic microorganism comprises or consists of an ethane monooxygenase, an ethylotrophy pathway, and/or a malonyl-CoA-product pathway. In some embodiments, the single microorganism is Escherichia coli.

### D. Genomic Integration of Exogenous Polynucleotides

In some embodiments, the at least one exogenous polynucleotide is inserted or integrated into the genome of the microorganism. One skilled in the art would have expected that the addition of three exogenous plasmids would show greater expression than two integrated copies. However, counterintuitively, greater production of an end product can be achieved in some instances with a small number of copies of a polypeptide-encoding polynucleotide integrated into the host genome than with the same polynucleotide in a plasmid with higher copy number. For example, as can be seen from the examples, two copies of ethane monooxygenase operons integrated into an E. Coli host genome led to production of more 3HP than plasmid-borne ethane monooxygenase operons as set forth in the examples.

### E. Ethane Monooxygenase

In some embodiments, the at least one exogenous polynucleotide encodes one or more polypeptides comprising or consisting of an ethane monooxygenase. An ethane monooxygenase, as contemplated in the current invention, is an enzyme that can oxidize a two-carbon molecule such as, for example, without limitation, ethane. In some embodiments, the ethane monooxygenase comprises or consists of a methane monooxygenase such as, for example, sMMO and/or pMMO. In some embodiments, an ethane monooxygenase comprises or consists of a butane monooxygenase. In some embodiments, an ethane monooxygenase comprises or consists of a propane monooxygenase. In some embodiments, an ethane monooxygenase comprises or consists of an ammonium monooxygenase. In some embodiments, an ethane monooxygenase comprises or consists of a P450. In some embodiments, an ethane monooxygenase comprises or consists of AlkB.

In some embodiments, the ethane monooxygenase comprises or consists of one or more methane monooxygenase(s). The methane monooxygenases are enzymes complexes capable of oxidizing a carbon-hydrogen bond of the methane molecule and they can also often oxidize ethane to ethanol. Naturally occurring methane-consuming microorganisms have evolved at least two classes of methane monooxygenase enzymes: soluble monooxygenases (sMMO) and particulate monooxygenases (pMMO). Any enzyme or enzyme complex of these categories, any mutated enzyme or complex, or any researcher-designed enzyme or enzyme complex that converts methane into methanol would be considered a methane monooxygenase enzyme. Many of these enzymes are known to also oxidize a wide range of substrates, such as methane to methanol or ethane into ethanol, and thus, are relevant as embodiments of the invention.

In some embodiments, an ethane monooxygenase comprises or consists of an sMMO. The sMMO from Methylococcus capsulatus (Bath) is well-studied. The Methylococcus capsulatus (Bath) can hydroxylate a large number of substrates (See, Petroleum Biotechnology by Vazquez-Duhalt and Quintero-Romero in 2004, which is incorporated by reference in its entirety herein). The sMMO from Methylococcus capsulatus (Bath) is able to hydroxylate dozens of substrates into an even larger number of products, when assayed in vitro. In some embodiments, the ethane monooxygenase comprises or consists of the monooxygenase from Methylococcus capsulatus (Bath).

In some embodiments, the ethane monooxygenase comprises or consists of a methane monooxygenase from one or more Methylomonas methanica, Methylocaldum sp. 175, Methyloferula stellata, Methylocystis LW5, Solimonas aquatica (DSM 25927), Methylovulum miyakonense, Rhodococcus ruber IGEM 231, and/or Conexibacter woesei.

In some embodiments, the ethane monooxygenase comprises or consists of a monooxygenase as set forth in the following table:

| Organism | Gene names | Accession number |
|---|---|---|
| Pseudomonas mendocina | tmoABCDEF | AY552601.1 |
| KR1 | | |
| Methylocella silvestris | Msil1651-1647 | NC_011666.1 |
| BL2 | | |
| Mycobacterium NBB4 | smoXYC1B1Z, groL | CP003054.1 |
| | (Mycch_5901 - | |
| | Mycch_5897, | |
| | Mycch_5390) | |
| Thauera butanivorans | bmoXYBZDC | AAM19732.1, |
| | | AAM19731.1, |
| | | AAM19730.1, |
| | | AAM19729.1, |
| | | AAM19728.1, |
| | | AAM19727.1, |
| | | ABU68845.2 |
| Mycobacterium | mimABCD | CP000480.1 |
| smegmatis mc2-155 | | |
| Gordonia TY-5 | prmABCDG | AB112920.1 |
| Pseudonocardia | WP_037052656.1 to | NZ_{—} |
| autotrophica | WP_037052662.1 | JNYD01000036.1 |
| Amycolatopsis | AMETH_2368-2375 | CP009110.1 |
| methanolica 239 | | |
| Mycobacterium | CYP153A6 (ahpGHI) | AJ783967.1 |
| HXN-1500 | | |
| Bacillus megaterium | P450-BM3 | WP_034650526.1 |
| Pseudomonas putida | P450cam | WP_032492633.1 |
| Methylocella silvestris | mmoXYBZDC | NC_011666.1 |
| BL2 | (Msil1262-Msil1267) | |
| Methylococcus | mmoXYBZDC_G | AF525283.1, |
| capsulatus (Bath) | | M90050.3 |
| Methylosinus | mmoXYBZDC, groEL | X55394.3, |
| trichosporium OB3b | | EF685207.1 |
| Methylococcus | pmoCAB | L40804.2 |
| capsulatus (Bath) | | |
| Methylosinus | pmoCAB | U31650.2 |
| trichosporium OB3b | | |
| Pseudomonas putida | alkBFGHJKLNST | NG_035191.1 |
| (OCT plasmid) | | |
| Rhodococcus corallinus | amoABCD | D37875.1 |
| B-276 | | |

The particulate methane monooxygenase (pMMO) may also be used to oxidize ethane to ethanol. This protein complex is composed of three subunits and resides in the inner membrane of the native organism. In some embodiments, the ethane monooxygenase comprises or consists of a pMMO.

In some embodiments, the ethane monooxygenase comprises or consists of a butane monooxygenase. The butane monooxygenase of Thauera butanivorans is most active on butane, and maintains some activity against shorter alkanes. In some embodiments, the ethane monooxygenase comprises or consists of the butane monooxygenase of Thauera butanivorans (See, Sluis et al, Molecular analysis of the soluble butane monooxygenase from Pseudomonas butanovora, Microbiology, 148, 3617-3629 (2002), which is incorporated by reference in its entirety herein). The monooxygenase from Pseudomonas putida has also shown bacterial oxidation for propane and butane (See, Johnson, E. et al, Propane and n-Butane oxidation by Pseudomonas putida GPo1, Applied and environmental microbiology, 950-952 (2006), which is incorporated by reference in its entirety herein).

In some embodiments, the ethane monooxygenase comprises or consists of a propane monooxygenase. The propane monooxygenase of Pseudonocardia or Mycobacterium may be useful with shorter alkanes (See, for example, Furuya, et al., The mycobacterial iron monooxygenases require a specific chaperon-like protein for function expression in a heterologous host, FEBS Journal, 280, 817-826 (2013), which is incorporated by reference in its entirety herein). In some embodiments, the ethane monooxygenase comprises or consists of the propane monooxygenase from Mycobacterium NBB3 or from Mycobacterium NBB4 or the propane monooxygenase from Pseudonocardia TY-7 (See, for example, T. Kotani et al., Gene structure and regulation of alkane monooxygenase in propane utilizing Mycobacterium sp. TY-6 and Pseudonocardia sp. TY-7, Journal of bioscience and bioengineering, Vol. 102, No. 3, 184-192 (2006); and Martin, K. et al, SmoXYB1C1Z of Mycobacterium sp. Strain NBB4: a soluble methane monooxygenase (sMMO)-like enzyme, active on C2 and C4 alkanes and alkenes, Applied and Environmental Microbiology, p. 5801-5806, Vol. 80, No. 18 (2014), each of which is incorporated by reference in its entirety herein).

In some embodiments, the ethane monooxygenase comprises or consists of the propane monooxygenase from Gordonia (See, for example, T. Kotani et al, Propane monooxygenase and NAD+-dependent secondary alcohol dehydrogenase in propane metabolism by Gordonia sp. Strain TY-5, which is incorporated by reference herein in its entirety). In some embodiments, the ethane monooxygenase comprises or consists of a monooxygenase from any Gram-positive organism.

Ammonium monooxygenase (AMO) can also be useful according to the invention. Ammonia monooxygenase can oxidize ammonium and possibly ethane (See, Tavormina et al., A novel family of functional operons encoding methane/ammonia monooxygenase-related proteins in gammaproteobacterial methanotrophs, Environmental Microbiology reports (2011) 3(1), 91-100, which is incorporated by reference in its entirety herein). In some embodiments, the ethane monooxygenase comprises or consists of an ammonia monooxygenase.

In some embodiments, the ethane monooxygenase comprises or consists of P450. P450 may be engineered, according to the invention, to oxidize ethane (See F. Xu et al., The Heme Monooxygenase Cytochrome P450, 4029-4032, 2005; and P. Meinhold et al., Direct Conversion of Ethane to Ethanol by Engineered Cytochrome, 0017 1765-1768, 2005, each of which is incorporated by reference herein). In some embodiments, the ethane monooxygenase comprises or consists of P450 BM-3 (See, Arnold, F. et al, A Panel of Cytochrome P450 BM3 variants to produce drug metabolites and diversify lead compounds, Chemistry. 2009 Nov. 2; 15(43): 11723-11729, which is incorporated by reference in its entity herein). Cytochrome P450 BM3 is a Prokaryote Cytochrome P450 enzyme originally from Bacillus megaterium, catalyzes the hydroxylation of several long-chain fatty acids at the ω-1 through ω-3 positions. This bacterial enzyme belongs to CYP family CYP102, with the CYP Symbol CYP102A1. This CYP family constitutes a natural fusion between the CYP domain and an NADPH-dependent cytochrome P450 reductase. In some embodiments, the ethane monooxygenase comprises or consists of P450cam (See, for example, Sevrioukova et al., Structural Biology of Redox Partner Interactions in P450cam Monooxygenase: A Fresh Look at an Old System, Arch Biochem Biophys. 2011 Mar. 1; 507 (1): 66-74, which is incorporated by reference herein in its entirety).

In some embodiments, the ethane monooxygenase comprises or consists of a pMMO. The particulate methane monooxygenase (pMMO) may also oxidize ethane to ethanol. This protein complex is composed of three subunits and resides in the inner membrane of the native organism. To successfully express the pMMO in E. coli, correct N-terminal leader sequences must be properly fused to each of the three subunits. In some embodiments, the ethane monooxygenase comprises or consists of the pMMO from Methylococcus capsulatus (See, Elliot, S. et al, Regio- and Stereo selectivity of particulate methane monooxygenase from Methylococcus capsulatus (Bath), J. Am. Chem. Soc. 119, 9949-9955 (1997), which is incorporated by reference in its entirety herein).

AlkB may also be useful as an ethane monooxygenase of the invention. In some embodiments, the ethane monooxygenase comprises or consists of alkB (See, for example, Nie, Y, Diverse alkane hydroxylase genes in microorganisms and environments, Scientific Reports volume 4, Article number: 4968 (2014); and Koch, D. et al., In Vivo evolution of butane oxidation by terminal alkane hydroxylases AlkB and CYP153A6, applied and environmental biology, 337-344, (2009), each of which is incorporated by reference herein in its entirety). The integral-membrane alkane monooxygenase (AlkB)-related alkane hydroxylases are so-far the most commonly found alkane hydroxylases distributed in both Gram-negative and Gram-positive bacteria. In some embodiments, the ethane monooxygenase comprises or consists of alkB.

In some embodiments, the ethane monooxygenase comprises or consists of an ethane monooxygenase having an amino acid sequence that is more than about 70%, more than about 75%, more than about 80%, more than about 85%, more than about 90%, or more than about 95% identical or identical to any of SEQ ID NOs: 1-16.

### F. Ethylotrophy Pathway

In some embodiments, the at least exogenous polynucleotide encodes one or more polypeptides comprising or consisting of an ethylotrophy pathway. An ethylotrophy pathway utilizes reduced carbon substrates containing a single carbon-carbon bond, such as, for example, without limitation, ethane, ethanol, acetaldehyde, acetate, ethylene, and monoethylene glycol. One skilled in the art would understand that any ethylotrophy pathway may be used.

In some embodiments, the ethylotrophy pathway comprises or consists one or more alcohol dehydrogenases. In some embodiments, the one or more polypeptides comprise an alcohol dehydrogenase, for example adhA (EC 1.1.1.1). adhA catalyzes the reaction: ethanol+NAD⁺←→acetaldehyde+NADH. In some embodiments, adhA comprises or consists of an adhA from Corynebacterium glutamicum. In some embodiments, the ethylotrophy pathway comprises or consists of one or more aldehyde dehydrogenases, for example adhH (EC 1.2.1.10). adhH catalyzes the reaction: acetaldehyde+CoA+NAD⁺←Θacetyl-CoA+NADH. In some embodiments, adhH comprises or consists of an adhH from Corynebacterium glutamicum.

In some embodiments, the ethylotrophy pathway comprises or consists of one or more enzymes having an amino acid sequence that is more than about 70%, more than about 75%, more than about 80%, more than about 85%, more than about 90%, or more than about 95% identical or identical to any of SEQ ID NOs: 22-23.

### G. Malonyl-CoA-Product Pathway

In some embodiments, the at least exogenous polynucleotide encodes one or more polypeptides comprising or consisting of a malonyl-CoA-product pathway. A malonyl-CoA-product pathway uses malonyl-CoA as a reactant or intermediate molecule to produce a product of interest, such as, for example, without limitation, malonic acid, 3-hydroxypropionic acid, fatty acids, 3-hydroxyfatty acids, fatty alcohols, fatty esters, adipic acid, 2-butanone, 3-pentanone, or salts or derivatives thereof (see, for example, https://agilebiofoundry.org/wp-content/uploads/2020/09/ABF-Metabolic-Map-12-for-survey.pdf for products derived from malonyl-CoA as Beachhead Molecule 11, which is incorporated by reference in its entirety herein). In some embodiments, the one or more polypeptides comprising or consisting of a malonyl-CoA-product pathway comprise or consist of one or more of mcrC*** and/or mcrN from Chloroflexus aurantiacus (See, Liu et al., “Functional balance between enzymes in malonyl-CoA pathway for 3-hydroxypropionate biosynthesis”, Metabolic Engineering, Vol. 34, p. 104-110, (2016); and Lama et al., Production of a 3-hydroxypropionate acid from acetate using metabolically-engineered and glucose-grown Escherichia coli, Bioresource Technology, Vol. 320, Part A, 124362 (2021), each of which is incorporated by reference in herein in their entirety).

In some embodiments, the malonyl-CoA-product pathway comprises or consists of one or more enzymes having an amino acid sequence that is more than about 70%, more than about 75%, more than about 80%, more than about 85%, more than about 90%, or more than about 95% identical or identical to any of SEQ ID NOs: 29-30.

### H. Chaperones

Protein folding chaperones are proteins that improve the folding of polypeptide (amino acid) chains into 3-dimensional structures. Protein folding chaperones help their substrates, namely other proteins, to become properly folded and often more highly soluble. Since most proteins must be folded in a particular shape to be functional, the expression of protein folding chaperones can assist in the proper assembly of certain enzymes in a cell and thereby can result in an increase in the enzymatic activity of the substrate proteins.

In some embodiments, the at least one exogenous polynucleotide comprises or consists of one or more modifications. In some embodiments, the one or more modifications comprises or consists of polynucleotides encoding, and capable of expressing, one or more chaperone proteins. In some embodiment, the one or more chaperones comprises or consists of groEL and/or groES. In some embodiments, the groEL and/or groES are E. coli groEL and/or groES, M. capsulatus groEL and/or groES, or both. In some embodiments, the one or more chaperones comprise one or more polypeptides, each of the one or more polypeptides having an amino acid sequence, the amino acid sequence being more than about 70%, more than about 75%, more than about 80%, more than about 85%, more than about 90%, or more than about 95% identical or identical to any of one of SEQ ID NOs: 36-39, respectively.

### I. Method of Producing a Product

A second aspect provides a method of producing a product, the method comprising or consisting of culturing a synthetic microorganism comprising or consisting of at least one exogenous polynucleotide that encodes one or more polypeptides comprising or consisting of an ethane monooxygenase, an ethylotrophy pathway, and/or a malonyl-CoA-product pathway for a suitable period of time and under conditions sufficient to lead to improved production of the product. The at least one exogenous polynucleotide may be inserted or integrated into the genome of the microorganism.

The ethane monooxygenase comprises or consists of an ethane monooxygenase having an amino acid sequence that is more about 70%, more than about 75%, more than about 80%, more than about 85%, more than about 90%, or more than about 95% identical or identical to any of SEQ ID Nos: 1-16. The ethylotrophy pathway comprises or consists of one or more enzymes having an amino acid sequence that is more than about 70%, more than about 75%, more than about 80%, more than about 85%, more than about 90%, or more than about 95% identical or identical to any of SEQ ID Nos: 22-23. The malonyl-CoA-product pathway comprises or consists of one or more enzymes having an amino acid sequence that is more than about 70%, more than about 75%, more than about 80%, more than about 85%, more than about 90%, or more than about 95% identical or identical to any of SEQ ID Nos: 29-30.

### J. Product(s)

Synthetic organisms may make valuable products from substrates as set forth herein. The synthetic microorganism generates a product comprising or consisting of 3-hydroxypropionic acid. The synthetic microorganism may generate a product comprising or consisting of a polymer of 3-hydroxypropionic acid. The synthetic microorganism may generate one or more products comprising or consisting of fatty acids or fatty acid derivatives. The synthetic microorganism may generate a product comprising or consisting of polymer(s) of 3-hydroxy-fatty acids. Finally, the synthetic microorganism may generate a product comprising or consisting of malonic acid.

### K. Expression of Nucleic Acid(s)

Expression of one or more exogenous nucleic acids in a microorganism or synthetic culture can be accomplished by introducing into a microorganism or culture a nucleic acid comprising a nucleotide sequence encoding the one or more polypeptides under the control of regulatory elements that permit expression in the microorganism or culture.

Nucleic acids encoding the one or more polypeptides can be introduced into a microorganism or culture by any method known to one of skill in the art without limitation (See, for example, Hinnen et al. (1978) Proc. Natl. Acad. Sci. USA 75:1292-3; Cregg et al. (1985) Mol. Cell. Biol. 5:3376-3385; Goeddel et al. eds, 1990, Methods in Enzymology, vol. 185, Academic Press, Inc., CA; Krieger, 1990, Gene Transfer and Expression—A Laboratory Manual, Stockton Press, NY; Sambrook et al., 1989, Molecular Cloning—A Laboratory Manual, Cold Spring Harbor Laboratory, NY; and Ausubel et al., eds., Current Edition, Current Protocols in Molecular Biology, Greene Publishing Associates and Wiley Interscience, NY, each of which is incorporated by reference in its entirety herein). Exemplary techniques include, but are not limited to, spheroplasting, electroporation, PEG 1000 mediated transformation, and lithium acetate- or lithium chloride-mediated transformation. In some embodiments, the nucleic acid is an extrachromosomal plasmid. In some embodiments, the nucleic acid is a chromosomal integration vector that can integrate the nucleotide sequence into the chromosome of the microorganism or culture.

### L. Modification

Expression of genes and genomes may be modified. In some embodiments, expression of the one of more exogenous or endogenous nucleic acids is modified. For example, the copy number of an enzyme or one or more polypeptides in a microorganism or culture may be altered by modifying the transcription of the gene that encodes the enzyme or one or more polypeptides. This can be achieved, for example, by modifying the copy number of the nucleotide sequence encoding the enzyme or one or more polypeptides (e.g., by using a higher or lower copy number expression vector comprising the nucleotide sequence, or by introducing additional copies of the nucleotide sequence into the genome of the microorganism or culture, or by introducing additional nucleotide sequences into the genome of the microorganism or culture that express the same or similar polypeptide, or by genetically modifying or deleting or disrupting the nucleotide sequence in the genome of the microorganism or culture), by changing the order of coding sequences on a polycistronic mRNA of an operon, or by breaking up an operon into individual genes, each with its own control elements. The strength of the promoter, enhancer, or operator to which the nucleotide sequence is operably linked may also be manipulated or increased or decreased or different promoters, enhancers, or operators may be introduced.

Counterintuitively, greater production of an end product can be achieved in some instances with a small number of copies of a polypeptide-encoding polynucleotide integrated into the host genome than with the same polynucleotide in a plasmid with higher copy number. For example, as can be seen from the examples, two copies of ethane monooxygenase operons integrated into an E. Coli host genome led to production of more 3HP than plasmid-borne methane monooxygenase operons as set forth in the examples.

Alternatively, or in addition, the copy number of the one or more polypeptides may be altered by modifying the level of translation of an mRNA that encodes the one or more polypeptides. This can be achieved, for example, by modifying the stability of the mRNA, modifying the sequence of the ribosome binding site, modifying the distance or sequence between the ribosome binding site and the start codon of the enzyme coding sequence, modifying the entire intercistronic region located upstream of or adjacent to the 5′ side of the start codon of the enzyme coding region, stabilizing the 3′-end of the mRNA transcript using hairpins and specialized sequences, modifying the codon usage of an enzyme, altering expression of rare codon tRNAs used in the biosynthesis of the enzyme, and/or increasing the stability of an enzyme, as, for example, via mutation of its coding sequence.

Expression of exogenous or endogenous nucleic acids may be modified or regulated by targeting particular genes. For example, without limitation, in some embodiments of the methods described herein, a microorganism, culture, or synthetic microorganism is contacted with one or more nucleases capable of cleaving, i.e., causing a break at a designated region within a selected site. In some embodiments, the break is a single-stranded break, that is, one but not both strands of the target site is cleaved. In some embodiments, the break is a double-stranded break. In some embodiments, a break-inducing agent is used. A break-inducing agent is any agent that recognizes and/or binds to a specific polynucleotide recognition sequence to produce a break at or near a recognition sequence. Examples of break-inducing agents include, but are not limited to, endonucleases, site-specific recombinases, transposases, topoisomerases, and zinc finger nucleases, and include modified derivatives, variants, and fragments thereof.

In some embodiments, a recognition sequence within a selected target site can be endogenous or exogenous to a microorganism, culture, or synthetic microorganism's genome. When the recognition site is an endogenous or exogenous sequence, it may be a recognition sequence recognized by a naturally occurring, or native break-inducing agent. Alternatively, an endogenous or exogenous recognition site could be recognized and/or bound by a modified or engineered break-inducing agent designed or selected to specifically recognize the endogenous or exogenous recognition sequence to produce a break. In some embodiments, the modified break-inducing agent is derived from a native, naturally occurring break-inducing agent. In other embodiments, the modified break-inducing agent is artificially created or synthesized. Methods for selecting such modified or engineered break-inducing agents are known in the art.

In some embodiments, the one or more nucleases is a CRISPR/Cas-derived RNA-guided endonuclease. CRISPR may be used to recognize, genetically modify, and/or silence genetic elements at the RNA or DNA level or to express heterologous or homologous genes. CRISPR may also be used to regulate endogenous or exogenous nucleic acids. Any CRISPR/Cas system known in the art finds use as a nuclease in the methods and compositions provided herein. CRISPR systems that find use in the methods and compositions provided herein also include those described in International Publication Numbers WO 2013/142578 A1, WO 2013/098244 A1 and Nucleic Acids Res (2017) 45 (1): 496-508, the contents of which are hereby incorporated in their entireties.

In some embodiments, the one or more nucleases is a TAL-effector DNA binding domain-nuclease fusion protein (TALEN). TAL effectors of plant pathogenic bacteria in the genus Xanthomonas play important roles in disease, or trigger defense, by binding host DNA and activating effector-specific host genes. (See, e.g., Gu et al. (2005) Nature 435:1122-5; Yang et al., (2006) Proc. Natl. Acad. Sci. USA 103:10503-8; Kay et al., (2007) Science 318:648-51; Sugio et al., (2007) Proc. Natl. Acad. Sci. USA 104:10720-5; Romer et al., (2007) Science 318:645-8; Boch et al., (2009) Science 326 (5959): 1509-12; and Moscou and Bogdanove, (2009) 326 (5959): 1501, each of which is incorporated by reference in their entirety). A TAL effector comprises a DNA binding domain that interacts with DNA in a sequence-specific manner through one or more tandem repeat domains. The repeated sequence typically comprises 34 amino acids, and the repeats are typically 91-100% homologous with each other. Polymorphism of the repeats is usually located at positions 12 and 13, and there appears to be a one-to-one correspondence between the identity of repeat variable-diresidues at positions 12 and 13 with the identity of the contiguous nucleotides in the TAL-effector's target sequence.

The TAL-effector DNA binding domain may be engineered to bind to a desired sequence and fused to a nuclease domain, e.g., from a type II restriction endonuclease, typically a nonspecific cleavage domain from a type II restriction endonuclease such as FokI (See, e.g., Kim et al. (1996) Proc. Natl. Acad. Sci. USA 93:1156-1160, which is incorporated by reference in its entirety herein). Other useful endonucleases may include, for example, HhaI, HindIII, Nod, BbvCI, EcoRI, BglI, and AlwI. Thus, in some embodiments, the TALEN comprises a TAL effector domain comprising a plurality of TAL effector repeat sequences that, in combination, bind to a specific nucleotide sequence in a target DNA sequence, such that the TALEN cleaves target DNA within or adjacent to the specific nucleotide sequence. TALENS useful for the methods provided herein include those described in WO10/079430 and U.S. Patent Application Publication No. 2011/0145940, which is incorporated by reference herein in its entirety.

In some embodiments, the one or more of the nucleases is a zinc-finger nuclease (ZFN). ZFNs are engineered break-inducing agents comprised of a zinc finger DNA binding domain and a break-inducing agent domain. Engineered ZFNs consist of two zinc finger arrays (ZFAs), each of which is fused to a single subunit of a non-specific endonuclease, such as the nuclease domain from the FokI enzyme, which becomes active upon dimerization.

Useful zinc-finger nucleases include those that are known and those that are engineered to have specificity for one or more sites. Zinc finger domains are amenable for designing polypeptides which specifically bind a selected polynucleotide recognition sequence. Thus, they are amenable to modifying or regulating expression by targeting particular genes.

In some embodiments, the activity of one or more genes native to the microorganism, culture, or synthetic microorganism is modified. The activity of one or more genes native to the microorganism or synthetic culture can be modified in a number of other ways, including, but not limited to, gene silencing or any other form of genetic modification, expressing a modified form of the polypeptides or one or more polypeptides that exhibits increased or decreased solubility in the microorganism, culture, or synthetic microorganism, expressing an altered form of the polypeptides or one or more polypeptides that lacks a domain through which the activity of a polypeptide is inhibited, expressing a modified form of the polypeptides that has a higher or lower k_{cat} or a lower or higher Kₘ for a substrate, or expressing an altered form of the one or more polypeptides or protein product of one or more genes native to the microorganism, culture, or synthetic microorganism that is more or less affected by feed-back or feed-forward regulation by another molecule in the pathway.

### M. Nucleic Acid(s)

A third aspect provides a nucleic acid suitable for integration into a microorganism, wherein the nucleic acid comprises or consists of at least one polynucleotide that encodes one or more polypeptides comprising or consisting of an ethane monooxygenase, an ethylotrophy pathway, and/or a malonyl-CoA-product pathway. The at least one polynucleotide may be inserted or integrated into the genome of a microorganism.

The ethane monooxygenase comprises or consists of an ethane monooxygenase having an amino acid sequence that is more than about 70%, more than about 75%, more than about 80%, more than about 85%, more than about 90%, or more than about 95% identical or identical to any of SEQ ID Nos: 1-16. The ethylotrophy pathway comprises or consists of one or more enzymes having an amino acid sequence that is more than about 70%, more than about 75%, more than about 80%, more than about 85%, more than about 90%, or more than about 95% identical or identical to any of SEQ ID Nos: 22-23. The malonyl-CoA-product pathway comprises or consists of one or more enzymes having an amino acid sequence that is more than about 70%, more than about 75%, more than about 80%, more than about 85%, more than about 90%, or more than about 95% identical or identical to any of SEQ ID Nos: 29-30.

Any suitable microorganism may be used with the nucleic acid. In some embodiments, the synthetic microorganism is derived from Escherichia coli.

In some embodiments, the one or more polypeptides or one or more genes native to the microorganism, culture, or synthetic microorganism are modified. It will be recognized by one skilled in the art that absolute identity to the one or more polypeptides or one or more genes native to the microorganism, culture, or synthetic microorganism is not strictly necessary. For example, changes in a particular gene or polynucleotide comprising a sequence encoding a polypeptide or the one or more polypeptides can be performed and screened for activity. Such modified or mutated polynucleotides and polypeptides can be screened for expression or function using methods known in the art.

Those of skill in the art will recognize that, due to the degenerate nature of the genetic code, a variety of polynucleotides differing in their nucleotide sequences can be used to encode one or more genes native to the microorganism, culture, or synthetic microorganism or one or more polypeptides of the disclosure. Due to the inherent degeneracy of the genetic code, other polynucleotides, which encode substantially the same or functionally equivalent polypeptides, can also be used. The disclosure includes polynucleotides of any sequence that encode the amino acid sequences of the polypeptides and proteins of the one or more polypeptides utilized in the methods of the disclosure.

In similar fashion, a polypeptide can typically tolerate one or more amino acid substitutions, deletions, and insertions in its amino acid sequence without loss or significant loss of a desired activity. The disclosure includes such one or more polypeptides with different amino acid sequences than the specific proteins described herein so long as the modified or variant polypeptides have an activity that is identical or similar to the referenced polypeptide. Accordingly, the amino acid sequences encoded by the polynucleotide sequences shown herein merely illustrate embodiments of the disclosure.

The disclosure also includes one or more polypeptides with different amino acid sequences than the specific proteins described herein if the modified or variant polypeptides have an activity that is desirable yet different from referenced polypeptide. In some embodiments, an enzyme may be altered by modifying the gene that encodes the enzyme so that the expressed protein is more or less active than the wild type version. As an example, any of the ethane monooxygenases, an ethylotrophy pathway polypeptides, and/or a malonyl-CoA-product pathway polypeptides may be more or less active according to substitutions.

As will be understood by those of skill in the art, it can be advantageous to modify a coding sequence to enhance expression in a particular host, such as, without limitation, Escherichia coli. The genetic code is redundant with 64 possible codons, but most organisms typically use a subset of these codons. Codons can be substituted, without any resultant change to the amino acid sequence of the corresponding protein, to increase or decrease the translation rate of the sequence, in a process sometimes called “codon optimization”.

Optimized coding sequences can be prepared, for example, to increase the rate of translation or to produce recombinant RNA transcripts having desirable properties, such as a longer half-life, as compared with transcripts produced from a non-optimized sequence. Translation stop codons can also be modified to reflect host preference.

In addition, homologs of enzymes or the one or more polypeptides or the proteins encoded by the one or more genes native to the microorganism, culture, or synthetic microorganism useful for the compositions and methods provided herein are encompassed by the disclosure. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences.

It is recognized that residue positions that are not identical often differ by conservative amino acid substitutions. In cases where two or more amino acid sequences differ from each other by conservative substitutions, the percent sequence identity or degree of homology may practically be adjusted upwards to correct for the conservative nature of the substitution. Means for making this adjustment are well known to those of skill in the art (See, e.g., Pearson W. R., 1994, Methods in Mol Biol 25:365-89, which is incorporated in its entirety herein).

Sequence homology and sequence identity for polypeptides is typically measured using sequence analysis software. A typical algorithm used to compare a molecular sequence to a database containing a large number of sequences from different organisms is the computer program BLAST. When searching a database containing sequences from a large number of different organisms, it is typical to compare amino acid sequences.

Furthermore, any of the one or more genes native to the microorganism, culture, or synthetic microorganism or genes encoding the enzymes or one or more polypeptides or genes native to the microorganism, culture, or synthetic microorganism (or any others mentioned herein (or any of the regulatory elements that control or modulate expression thereof)) may be optimized by genetic/protein engineering techniques, such as directed evolution or rational mutagenesis, which are known to those of ordinary skill in the art. Such action allows those of ordinary skill in the art to optimize the enzymes for expression and activity in yeast, bacteria, or any other suitable cell or organism.

For example, amino acid sequence variants of the one or more polypeptides can be prepared by mutations in the DNA. Methods for mutagenesis and nucleotide sequence alterations include, for example, Kunkel, (1985) Proc Natl Acad Sci USA 82:488-92; Kunkel, et al., (1987) Meth Enzymol 154:367-82; U.S. Pat. No. 4,873,192; Walker and Gaastra, eds. (1983) Techniques in Molecular Biology (MacMillan Publishing Company, New York) and the references cited therein. Guidance regarding amino acid substitutions not likely to affect biological activity of the protein is found, for example, in the model of Dayhoff, et al., (1978) Atlas of Protein Sequence and Structure (Natl Biomed Res Found, Washington, D.C.), each of which is incorporated by reference in their entirety.

Techniques known to those skilled in the art may be suitable to identify additional homologous (or otherwise analogous) genes and homologous (or otherwise analogous) enzymes. Generally, analogous genes and/or analogous enzymes can be identified by functional analysis and will have functional similarities. As an example, to identify homologous or analogous biosynthetic pathway genes, proteins, or enzymes, techniques may include, but are not limited to, cloning a gene by PCR using primers based on a published sequence of a gene/enzyme of interest or by degenerate PCR using degenerate primers designed to amplify a conserved region among a gene of interest.

Further, one skilled in the art can use techniques to identify homologous or analogous genes, proteins, or enzymes with functional homology or similarity. Techniques include examining a cell or cell culture for the catalytic activity of an enzyme through in vitro enzyme assays for the activity (e.g. as described herein or in Kiritani, K., Branched-Chain Amino Acids Methods Enzymology, 1970), then isolating the enzyme with the activity through purification, determining the protein sequence of the enzyme through techniques such as Edman degradation, design of PCR primers to the likely nucleic acid sequence, amplification of the DNA sequence through PCR, and cloning of the nucleic acid sequence. To identify homologous or similar genes and/or homologous or similar proteins, analogous genes and/or analogous proteins, techniques also include comparison of data concerning a candidate gene or enzyme with databases such as BRENDA, KEGG, or MetaCYC. The candidate gene or proteins may be identified within the above-mentioned databases in accordance with the teachings herein.

In some embodiments, the microorganism, culture, or synthetic microorganism expressing the one or more polypeptides has one or more genes native to the microorganism, culture, or synthetic microorganism that have been genetically modified, deleted, or whose expression has been reduced or eliminated. In some embodiments, the araBAD genes have been deleted.

Reduction or elimination of expression may occur through any method known to one skilled in the art and all ways of genetically modifying, deleting, and/or of reducing or eliminating expression of genes native to the microorganism, culture, or synthetic microorganism are provided herein. In particular, one skilled in the art will understand that any form of genetic alteration or genetic engineering or genetic modification, such as those set forth above related to expression, may be used as an alternative to deletion. In some embodiments, other forms of genetic modification that may be used as an alternative to deletion include, for example, without limitation, gene knockouts, mutation, gene targeting, homologous recombination, gene knockdown, gene silencing, gene addition, molecular cloning, gene attenuation, genome editing, CRISPR interference or any technique that may be used to suppress or alter or enhance a particular phenotype.

In some embodiments, the one or more genes native to the microorganism, culture, or synthetic microorganism can be altered in other ways, including, but not limited to, expressing a modified form of a polypeptide where the modified form of the polypeptide exhibits increased or decreased solubility in the microorganism or synthetic culture, expressing an altered form of a polypeptide that lacks a domain through which activity is inhibited, or expressing an altered form of a polypeptide that is more or less affected by feed-back or feed-forward regulation by another molecule in a pathway expressed in the microorganism, culture, or synthetic microorganism. In some embodiments, the strength of the promoter, enhancer, or operator to which the nucleotide sequence for the one or more genes native to the microorganism, culture, or synthetic microorganism is operably linked may also be manipulated, decreased, or increased or different promoters, enhancers, or operators may be introduced.

### S. Sequences

TABLE S

| SEQ | | | |
|---|---|---|---|
| ID | | Region and/or | |
| NO | Molecule | Designation | Sequence |
| 1. | mmoX | M. capsulatus | MALSTATKAATDALAANRAPTSGNAQEVHRWLQSFNW |
| | | (Bath) | DFKNNRTKYATKYKMANETKEQFKLIAKEYARMEAVK |
| | | | DERQFGSLQDALTRLNAGVRVHPKWNETMKVVSNFLE |
| | | | VGEYNAIAATGMLWDSAQAAEQKNGYLAQVLDEIRHT |
| | | | HQCAYVNYYFAKNGQDPAGHNDARRTRTIGPLWKGMK |
| | | | RVFSDGFISGDAVECSLNLQLVGEACFTNPLIVAVTE |
| | | | WAAANGDEITPTVFLSIETDELRHMANGYQTVVSIAN |
| | | | DPASAKYLNTDLNNAFWTQQKYFTPVLGMLFEYGSKF |
| | | | KVEPWVKTWNRWVYEDWGGIWIGRLGKYGVESPRSLK |
| | | | DAKQDAYWAHHDLYLLAYALWPGGFFRLALPDQEEME |
| | | | WFEANYPGWYDHYGKIYEEWRARGCEDPSSGFIPLMW |
| | | | FIENNHPIYIDRVSQVPFCPSLAKGASTLRVHEYNGQ |
| | | | MHTFSDQWGERMWLAEPERYECQNIFEQYEGRELSEV |
| | | | IAELHGLRSDGKTLIAQPHVRGDKLWTLDDIKRLNCV |
| | | | FKNPVKAFN* |
| 2. | mmoY | M. capsulatus | MSMLGERRRGLTDPEMAAVILKALPEAPLDGNNKMGY |
| | | (Bath) | FVTPRWKRLTEYEALTVYAQPNADWIAGGLDWGDWTQ |
| | | | KFHGGRPSWGNETTELRTVDWFKHRDPLRRWHAPYVK |
| | | | DKAEEWRYTDRFLQGYSADGQIRAMNPTWRDEFINRY |
| | | | WGAFLFNEYGLFNAHSQGAREALSDVTRVSLAFWGFD |
| | | | KIDIAQMIQLERGFLAKIVPGFDESTAVPKAEWTNGE |
| | | | VYKSARLAVEGLWQEVFDWNESAFSVHAVYDALFGOF |
| | | | VRREFFQRLAPRFGDNLTPFFINQAQTYFQIAKQGVQ |
| | | | DLYYNCLGDDPEFSDYNRTVMRNWTGKWLEPTIAALR |
| | | | DFMGLFAKLPAGTTDKEEITASLYRVVDDWIEDYASR |
| | | | IDFKADRDQIVKAVLAGLK* |
| 3. | mmoB | M. capsulatus | MSVNSNAYDAGIMGLKGKDFADQFFADENQVVHESDT |
| | | (Bath) | VVLVLKKSDEINTFIEEILLTDYKKNVNPTVNVEDRA |
| | | | GYWWIKANGKIEVDCDEISELLGRQFNVYDFLVDVSS |
| | | | TIGRAYTLGNKFTITSELMGLDRKLEDSHA* |
| 4. | mmoZ | M. capsulatus | MAKLGIHSNDTRDAWVNKIAQLNTLEKAAEMLKQFRM |
| | | (Bath) | DHTTPFRNSYELDNDYLWIEAKLEEKVAVLKAEAFNE |
| | | | VDFRHKTAFGEDAKSVLDGTVAKMNAAKDKWEAEKIH |
| | | | IGFRQAYKPPIMPVNYFLDGERQLGTRLMELRNLNYY |
| | | | DTPLEELRKQRGVRVVHLQSPH* |
| 5. | mmoD | M. capsulatus | MVESAFQPFSGDADEWFEEPRPQAGFFPSADWHLLKR |
| | | (Bath) | DETYAAYAKDLDFMWRWVIVREERIVQEGCSISLESS |
| | | | IRAVTHVLNYFGMTEQRAPAEDRTGGVQH* |
| 6. | mmoC | M. capsulatus | MQRVHTITAVTEDGESLRFECRSDEDVITAALRQNIF |
| | | (Bath) | LMSSCREGGCATCKALCSEGDYDLKGCSVQALPPEEE |
| | | | EEGLVLLCRTYPKTDLEIELPYTHCRISFGEVGSFEA |
| | | | EVVGLNWVSSNTVQFLLQKRPDECGNRGVKFEPGQFM |
| | | | DLTIPGTDVSRSYSPANLPNPEGRLEFLIRVLPEGRF |
| | | | SDYLRNDARVGQVLSVKGPLGVFGLKERGMAPRYFVA |
| | | | GGTGLAPVVSMVRQMQEWTAPNETRIYFGVNTEPELF |
| | | | YIDELKSLERSMRNLTVKACVWHPSGDWEGEQGSPID |
| | | | ALREDLESSDANPDIYLCGPPGMIDAACELVRSRGIP |
| | | | GEQVFFEKFLPSGAA* |
| 7. | pmoC/ | M. capsulatus | MAATTIGGAAAAEAPLLDKKWLTFALAIYTVFYLWVR |
| | ammonia | (Bath) | WYEGVYGWSAGLDSFAPEFETYWMNFLYTEIVLEIVT |
| | monooxygenase | | ASILWGYLWKTRDRNLAALTPREELRRNFTHLVWLVA |
| | (pMMO) | | YAWAIYWGASYFTEQDGTWHQTIVRDTDFTPSHIIEF |
| | | | YLSYPIYIITGFAAFIYAKTRLPFFAKGISLPYLVLV |
| | | | VGPFMILPNVGLNEWGHTFWFMEELFVAPLHYGFVIF |
| | | | GWLALAVMGTLTQTFYSFAQGGLGQSLCEAVDEGLIA |
| | | | K |
| 8. | P450 | Mycobacterium sp. | MTEMTVAASDATNAAYGMALEDIDVSNPVLFRDNTWH |
| | CYP153A6 | HXN-1500 | PYFKRLREEDPVHYCKSSMFGPYWSVTKYRDIMAVET |
| | | | NPKVFSSEAKSGGITIMDDNAAASLPMFIAMDPPKHD |
| | | | VQRKTVSPIVAPENLATMESVIRQRTADLLDGLPINE |
| | | | EFDWVHRVSIELTTKMLATLFDFPWDDRAKLTRWSDV |
| | | | TTALPGGGIIDSEEQRMAELMECATYFTELWNQRVNA |
| | | | EPKNDLISMMAHSESTRH |
| | | | MAPEEYLGNIVLLIVGGNDTTRNSMTGGVLALNEFPD |
| | | | EYRKLSANPALISSMVSEIIRWQTPLSHMRRTALEDI |
| | | | EFGGKHIRQGDKVVMWYVSGNRDPEAIDNPDTFIIDR |
| | | | AKPRQHLSFGFGIHRCVGNRLAELQLNILWEEILKRW |
| | | | PDPLQIQVLQEPTRVLSPFVKGYESLPVRINA |
| 9. | P450cam | Pseudomonas | MTTETIQSNANLAPLPPHVPEHLVFDFDMYNPSNLSA |
| | | putida | GVQEAWAVLQESNVPDLVWTRONGGHWIATRGQLIRE |
| | | | AYEDYR |
| | | | HFSSECPFIPREAGEAYDFIPTSMDPPEQRQFRALAN |
| | | | QVVGMPVVDKLENRIQELACSLIESLRPQGQCNFTED |
| | | | YAEPFPIRIFMLLAGLPEEDIPHLKYLTDQMTRPDGS |
| | | | MTFAEAKEALYDYLIPIIEQRROKPGTDAISIVANGQ |
| | | | VNGRPITSDEAKRMCGLLLVGGLDTVVNFLSFSMEFL |
| | | | AKSPEHRQELIERPERIPAACEELLRRFSLVADGRIL |
| | | | TSDYEFHGVQLKKGDQILLPQMLSGLDERENACPMHV |
| | | | DFSROKVSHTTFGHGSHLCLGQHLARREIIVTLKEWL |
| | | | TRIPDFSIAPGAQIQHKSGIVSGVQALPLVWDPATTK |
| | | | AV |
| 10.1 | P450cam w/ | Pseudomonas | MTTETIQSNANLAPLPPHVPEHLVFDFDMYNPSNLSA |
| | mutations | putida | GVQEAWAVLQESNVPDLVWTRONGGHWIATRGQLIRE |
| | for | | AYEDYRHFSSECPWIPREAGEAFDFIPLSMDPPEQRQ |
| | ethane | | FRALANQVVGMPVVDKLENRIQELACSLIESLRPQGQ |
| | | | CNFTEDYAEPFPIRIFMLLAGLPEEDIPHLKYLTDQM |
| | | | MRPDGSMTFAEAKEALYDYLIPIIEQRROKPGTDAIS |
| | | | IVANGQVNGRPITSDEAKRMCGMLLLAGLDTVVNFLS |
| | | | FSMEFLAKSPEHRQELIERPERIPAACEELLRRFSMV |
| | | | ADGRILTSDYEFHGVQLKKGDQILLPQMLSGLDEREN |
| | | | ACPMHVDFSRQKVSHTTFGHGSHLCPGQHLARREIIV |
| | | | TLKEWLTRIPDFSIAPGAQIQHKSGIVSGVQALPLVW |
| | | | DPATTKAV* |
| 11. | alkB | Pseudomonas | MLEKHRVLDSAPEYVDKKKYLWILSTLWPATPMIGIW |
| | | putida | LANETGWGIFYGLVLLVWYGALPLLDAMFGEDFNNPP |
| | | | EEVVPKLEKERYYRVLTYLTVPMHYAALIVSAWWVGT |
| | | | QPMSWLEIGALALSLGIVNGLALNTGHELGHKKETFD |
| | | | RWMAKIVLAVVGYGHFFIEHNKGHHRDVATPMDPATS |
| | | | RMGESIYKFSIREIPGAFIRAWGLEEQRLSRRGQSVW |
| | | | SFDNEILQPMIITVILYAVLLALFGPKMLVFLPIQMA |
| | | | FGWWQLTSANYIEHYGLLRQKMEDGRYEHQKPHHSWN |
| | | | SNHIVSNLVLFHLQRHSDHHAHPTRSYQSLRDFPGLP |
| | | | ALPTGYPGAFLMAMIPQWFRSVMDPKVVDWAGGDLNK |
| | | | IQIDDSMRETYLKKFGTSSAGHSSSTSAVA S |
| 12. | Propane | Gordonia TY-5 | MSRQSLTKAHAKITELSWEPTFATPATRFGTDYTFEK |
| | monooxygenase | prmA | APKKDPLKQIMRSYFPMEEEKDNRVYGAMDGAIRGNM |
| | | | FRQVQERWLEWOKLFLSIIPFPEISAARAMPMAIDAV |
| | | | PNPEIHNGLAVOMIDEVRHSTIQMNLKKLYMNNYIDP |
| | | | AGFDITEKAFANNYAGTIGRQFGEGFITGDAITAANI |
| | | | YLTVVAETAFTNTLFVAMPDEAAANGDYLLPTVFHSV |
| | | | QSDESRHISNGYSILLMALADERNRPLLERDLRYAWW |
| | | | NNHCVVDAAIGTFIEYGTKDRRKDRESYAEMWRRWIY |
| | | | DDYYRSYLLPLEKYGLTIPHDLVEEAWNRIVDKHYVH |
| | | | EVARFFATGWPVNYWRIDAMTDTDFEWFEEKYPGWYN |
| | | | KFGKWWENYNRLAYPGKNKPIAFEDVDYEYPHRCWTC |
| | | | MVPCLIREDMVTDKVDGQWRTYCSETCAWTDKVAFRP |
| | | | EYEGRPTPNMGRLTGFREWETLHHGKDLADIITDLGY |
| | | | VRDDGKTLIPQPHLDLDPKKMWTLDDVRGIPFGSPNV |
| | | | ALNEMSDDEREAHIAAYMANKNGAVTV |
| 13. | Propane | Pseudonocardia sp. | MSRQSMSKAHKKITELSWEPTFATPAKRFGTDYTFDN |
| | monooxygenase | TY-7 prmA | APKKDPLKQILRSYFPMEEEKDSRVFGAMDGAIRGNM |
| | | | FRQVQERWMEWQKLFLSIIPFPEISAARAMPMAIDAV |
| | | | PNPEIHNGLAVQMIDEVRHSTIQMNLKRLYMNYYIDP |
| | | | AGFDMTEKAFANNYAGTIGRQFGEGFITGDAITAANI |
| | | | YLTVVAETAFTNTLFVAMPSEAAANGDYLLPTVFHSV |
| | | | QSDESRHISNGYSILLMALSDEDNROLLERDLRYAWW |
| | | | NNHRVVDAAIGTFIEYGTKDRRKDRESYAEMWRRWIY |
| | | | DDYYRAYLIPLEKYGLVIPHDLIEESWKQIWEKGYVH |
| | | | EVAQFFATGWLANYWRIDSMTDEDFEWFEYKYPGWYD |
| | | | KYGKWWENYNRLSKPNGHNPIVFEDVDYVYPARCWTC |
| | | | MSPCWSVRTLVTAEVDGQHRTYCHEVCRWTDVRGFPS |
| | | | DVPGRETPNMGRLVGKREWETLYHGWNWADVVSDMGF |
| | | | VRDDGKTMTPKPHLDLDPKKMWTLDHMRRCPPLQSPN |
| | | | VLFNEMSDAERAAYVADYNKOGPAGRPAPQS |
| 14. | Propane | Mycobacterium | MTSTLPGKVSGHPSAPHASVSGQEVHSWLMDLGWDSD |
| | monooxygenase | NBB4 | TIRGKYPTKYKFDPNAREQFKLVARDYGRMEGEKDDR |
| | | | QYGSLLDSLARLKAPTRVEPRWAEVMKLLAGALELGE |
| | | | YNAIAGSAVLADTTRSPELRNGYLMQVEDEVRHTTQT |
| | | | HYLAKYYAGQYYDPAGFTDMRKWRYINPLFPPTMQAF |
| | | | GENFCAGDPVFASLNLQLVAEACFTNPLIVAMTEWSA |
| | | | ANGDEITPTIFLSIQSDEMRHMANGYQTIVSVAHDAD |
| | | | NMQYLQTDLENAFWLQHRFATPIVGAGFEYGAVNKLE |
| | | | PWAKVWDRWVYEDWGGIWLGRLEKFGVKSPANLADAK |
| | | | RQAYWGHHYTYAVAYAVWPLLGFRMDPPNARDMEWFE |
| | | | NNYPGWHSEVGHMWDSWREMGLADPANHTLPGOLVSD |
| | | | AKVPIYFCRVCNFPVIIPTLTGALDDVRILELNGRKH |
| | | | PLCSSWCERMFLKEPERYQGENLWEKFDGWNIADVVH |
| | | | AAGAVRSDGKTLLAQPHLNSERMWTLDDLRACHAVIR |
| | | | DPLKTGGVWLETV |
| 15. | Butane | Thauera | MSANMAVKQALKANPVPSSVDPQEVHKWLQDFTWDFK |
| | monooxygenase | butanivorans | GKTAKYPTKYEMDVNTREQFKLTAKEYARMESIKEER |
| | | | QYGTLLDGLDRLDAGNKVHPKWGEVMKLVSNFLETGE |
| | | | YGAIAGSALLWDTAQSPEQRNGYLAQVIDEIRHVNQT |
| | | | AYVNYYYGKHYYDPAGHTNMRQLRAINPLYPGVKRAF |
| | | | GEGFLAGDAVESSINLQLVGEACFTNPLIVSLTEWAA |
| | | | ANGDEITPTVFLSIETDELRHMANGYQTIVSIMNNPE |
| | | | TMKYLQTDLDNAFWTQHKFLTPFVGVALEYGSKYKVE |
| | | | PWAKSWNRWVYEDWAGIWLGRLQQFGVKTPKCLPDAK |
| | | | KDAVWAHHDLALLALALWPLTGIRMELPDSLAMEWFE |
| | | | ANYPGWYNHYGKIYEEWRAAGFEDPKSGFCGALWLME |
| | | | RGHGIFVDHASGLPFCPSLAKSSIKPRFTEYNGKRYA |
| | | | FAEPYGERQWLLEPERYEFQNFFEQFEGWELSDLVKA |
| | | | AGGVRSDGKTLIAQPHLRDTDMWTLDDLKRINLTIPD |
| | | | PMKILNWQPVAQ |
| 16. | AMO | Methylacidiphilum | MAQATTQTVSISIPERSAFSWKNLWIALAIITAFELA |
| | | infernorum | VNVYERTFAIAKGLDYFTPQYQTYWMSILYTELILEP |
| | | | TTLIALCSWLWVTRDRAMENLAPAEELKRYWNLGLFV |
| | | | VVYTVLLYWGASYYTEQDGTWHQTVIRDTDFTPSHII |
| | | | EFYQSYPIYIVAGVGSMVYAMTRLPQYAKAFSVPYAV |
| | | | LVGSPLMIFPNVGLNEFGHTRWFMEELFVAPLHWGFV |
| | | | MFGWGALAILGTWLQICPRVVELIKOVYYGKPATAPA |
| | | | VVLNDPEKAADPALCEI |
| 17. | | | |
| 18. | | | |
| 19. | | | |
| 20. | | | |
| 21 | | | |
| 22 | adhA | C. glutamicum | MTTAAPQEFTAAVVEKFGHDVTVKDIDLPKPGPHQAL |
| | | | VKVLTSGICHTDLHALEGDWPVKPEPPFVPGHEGVGE |
| | | | VVELGPGEHDVKVGDIVGNAWLWSACGTCEYCITGRE |
| | | | TQCNEAEYGGYTONGSFGQYMLVDTRYAARIPDGVDY |
| | | | LEAAPILCAGVTVYKALKVSETRPGQFMVISGVGGLG |
| | | | HIAVQYAAAMGMRVIAVDIADDKLELARKHGAEFTVN |
| | | | ARNEDSGEAVQKYTNGGAHGVLVTAVHEAAFGOALDM |
| | | | ARRAGTIVFNGLPPGEFPASVFNIVFKGLTIRGSLVG |
| | | | TRQDLAEALDFFARGLIKPTVSECSLDEVNGVLDRMR |
| | | | NGKIDGRVAIRF* |
| 23. | adhH | C. glutamicum | MTVYANPGTEGSIVNYEKRYENYIGGKWVPPVEGQYL |
| | | | ENISPVTGEVFCEVARGTAADVELALDAAHAAADAWG |
| | | | KTSVAERALILHRIADRMEEHLEEIAVAETWENGKAV |
| | | | RETLAADIPLAIDHFRYFAGAIRAQEDRSSQIDHNTV |
| | | | AYHFNEPIGVVGQIIPWNFPILMATWKLAPALAAGNA |
| | | | IVMKPAEQTPASILYLINIIGDLIPEGVLNIVNGLGG |
| | | | EAGAALSGSNRIGKIAFTGSTEVGKLINRAASDKIIP |
| | | | VTLELGGKSPSIFFSDVLSQDDAFAEKAVEGFAMFAL |
| | | | NQGEVCTCPSRALVHESIADEFLELGVKRVQNIKLGN |
| | | | PLDTETMMGAQASQEQMDKISSYLKIGPEEGAQTLTG |
| | | | GKVNKVDGMENGYYIEPTVFRGTNDMRIFREEIFGPV |
| | | | LSVATFSDFDEAIRIANDTNYGLGAGVWSRDQNTIYR |
| | | | AGRAIQAGRVWVNQYHNYPAHSAFGGYKESGIGRENH |
| | | | LMMLNHYQQTKNLLVSYDPNPTGLF* |
| 24. | | | |
| 25. | | | |
| 26 | | | |
| 27. | | | |
| 28. | | | |
| 29. | mcrC*** | C. aurantiacus | MSATTGARSASVGWAESLIGLHLGKVALITGGSAGIG |
| | | | GQIGRLLALSGARVMLAARDRHKLEQMOAMIQSELAE |
| | | | VGYTDVEDRVHIAPGCDVSSEAQLADLVERTLSAFGT |
| | | | VDYLINNAGIAGVEEMVIDMPVEGWRHTLFANLISNY |
| | | | SLMRKLAPLMKKQGSGYILNVSSYFGGEKDAAIPYPN |
| | | | RADYAVSKAGORAMAEVFARFLGPEIQINAIAPGPVE |
| | | | GDRLRGTGERPGLFARRARLILENKRLNELHAALIAA |
| | | | ARTDERSMHELVELLLPNDVAALEONPAAPTALRELA |
| | | | RRFRSEGDPAASSSSALLNRSIAAKLLARLHNGGYVL |
| | | | PADIFANLPNPPDPFFTRAQIDREARKVRDGIMGMLY |
| | | | LQRMPTEFDVAMATVYYLADRVVSGETFHPSGGLRYE |
| | | | RTPTGGELFGLPSPERLAELVGSTVYLIGEHLTEHLN |
| | | | LLARAYLERYGARQVVMIVETETGAETMRRLLHDHVE |
| | | | AGRLMTIVAGDQIEAAIDQAITRYGRPGPVVCTPFRP |
| | | | LPTVPLVGRKDSDWSTVLSEAEFAELCEHQLTHHFRV |
| | | | ARWIALSDGARLALVTPETTATSTTEQFALANFIKTT |
| | | | LHAFTATIGVESERTAQRILINQVDLTRRARAEEPRD |
| | | | PHERQQELERFIEAVLLVTAPLPPEADTRYAGRIHRG |
| | | | RAITV* |
| 30 | mcrN | C. aurantiacus | MSGTGRLAGKIALITGGAGNIGSELTRRFLAEGATVI |
| | | | ISGRNRAKLTALAERMQAEAGVPAKRIDLEVMDGSDP |
| | | | VAVRAGIEAIVARHGQIDILVNNAGSAGAQRRLAEIP |
| | | | LTEAELGPGAEETLHASIANLLGMGWHLMRIAAPHMP |
| | | | VGSAVINVSTIFSRAEYYGRIPYVTPKAALNALSQLA |
| | | | ARELGARGIRVNTIFPGPIESDRIRTVFQRMDQLKGR |
| | | | PEGDTAHHFLNTMRLCRANDQGALERRFPSVGDVADA |
| | | | AVFLASAESAALSGETIEVTHGMELPACSETSLLART |
| | | | DLRTIDASGRTTLICAGDQIEEVMALTGMLRTCGSEV |
| | | | IIGFRSAAALAQFEQAVNESRRLAGADFTPPIALPLD |
| | | | PRDPATIDAVFDWGAGENTGGIHAAVILPATSHEPAP |
| | | | CVIEVDDERVLNFLADEITGTIVIASRLARYWQSQRL |
| | | | TPGARARGPRVIFLSNGADONGNVYGRIQSAAIGOLI |
| | | | RVWRHEAELDYQRASAAGDHVLPPVWANQIVRFANRS |
| | | | LEGLEFACAWTAQLLHSQRHINEITLNIPANI* |
| 31. | | | |
| 32. | | | |
| 33. | | | |
| 34. | | | |
| 35. | | | |
| 36. | groES | E. coli | MNIRPLHDRVIVKRKEVETKSAGGIVLTGSAAAKSTR |
| | | | GEVLAVGNGRILENGEVKPLDVKVGDIVIFNDGYGVK |
| | | | SEKIDNEEVLIMSESDILAIVEA* |
| 37. | groEL | E. Coli | MAAKDVKFGNDARVKMLRGVNVLADAVKVTLGPKGRN |
| | | | VVLDKSFGAPTITKDGVSVAREIELEDKFENMGAQMV |
| | | | KEVASKANDAAGDGTTTATVLAQAIITEGLKAVAAGM |
| | | | NPMDLKRGIDKAVTAAVEELKALSVPCSDSKAIAQVG |
| | | | TISANSDETVGKLIAEAMDKVGKEGVITVEDGTGLQD |
| | | | ELDVVEGMQFDRGYLSPYFINKPETGAVELESPFILL |
| | | | ADKKISNIREMLPVLEAVAKAGKPLLIIAEDVEGEAL |
| | | | ATLVVNTMRGIVKVAAVKAPGFGDRRKAMLQDIATLT |
| | | | GGTVISEEIGMELEKATLEDLGQAKRVVINKDTTTII |
| | | | DGVGEEAAIQGRVAQIROQIEEATSDYDREKLQERVA |
| | | | KLAGGVAVIKVGAATEVEMKEKKARVEDALHATRAAV |
| | | | EEGVVAGGGVALIRVASKLADLRGQNEDQNVGIKVAL |
| | | | RAMEAPLRQIVLNCGEEPSVVANTVKGGDGNYGYNAA |
| | | | TEEYGNMIDMGILDPTKVTRSALQYAASVAGLMITTE |
| | | | CMVTDLPKNDAADLGAAGGMGGMM* |
| 38. | groES | M. capsulatus | VKIRPLHDRVIIKRLEEERTSAGGIVIPDSAAEKPMR |
| | | (Bath) | GEILAVGNGKVLDNGEVRALQVKVGDKVLFGKYAGTE |
| | | | VKVDGEDVVVMREDDILAVLES* |
| 39. | groEL-2* | M. capsulatus | MAKEVVYRGSARQRMMQGIEILARAAIPTLGATGPSV |
| | | (Bath) | MIQHRADGLPPISTRDGVTVANSIVLKDRVANLGARL |
| | | | LRDVAGTMSREAGDGTTTAIVLARHIAREMFKSLAVG |
| | | | ADPIALKRGIDRAVARVSEDIGARAWRGDKESVILGV |
| | | | AAVATKGEPGVGRLLLEALDAVGVHGAVSIELGORRE |
| | | | DLLDVVDGYRWEKGYLSPYFVTDRARELAELEDVYLL |
| | | | MTDREVVDFIDLVPLLEAVTEAGGSLLIAADRVHEKA |
| | | | LAGLLLNHVRGVFKAVAVTAPGFGDKRPNRLLDLAAL |
| | | | TGGRAVLEAQGDRLDRVTLADLGRVRRAVVSADDTAL |
| | | | LGIPGTEASRARLEGLRLEAEQYRALKPGQGSATGRL |
| | | | HELEEIEARIVGLSGKSAVYRVGGVTDVEMKERMVRI |
| | | | EGAYRSVVSALEEGVLPGGGVGFLGSMPVLAELEARD |
| | | | ADEARGIGIVRSALTEPLRIIGENSGLSGEAVVAKVM |
| | | | DHANPGWGYDQESGSFCDLHARGIWDAAKVLRLALEK |
| | | | AASVAGTFLTTEAVVLEIPDTDAFAGFSAEWAAATRE |
| | | | DPRV* |
| 40. | | | |
| 41. | | | |
| 42. | | | |
| 43. | | | |
| 44. | pNH284 | | TTGTTTCATCAAGCCTTACGGTCACCGTAACCAGCAA |
| | | | ATCAATATCACTGTGTGGCTTCAGGCCGCCATCCACT |
| | | | GCGGAGCCGTACAAATGTACGGCCAGCAACGTCGGTT |
| | | | CGAGATGGCGCTCGATGACGCCAACTACCTCTGATAG |
| | | | TTGAGTCGATACTTCGGCGATCACCGCTTCCCTCATA |
| | | | CTCTTCCTTTTTCAATATTATTGAAGCATTTATCAGG |
| | | | GTTATTGTCTCATGAGCGGATACATATTTGAATGTAT |
| | | | TTAGAAAAATAAACAAATAGCTAGCTCACTCGGTCGC |
| | | | TACGCTCCGGGCGTGAGACTGCGGCGGGCGCTGCGGA |
| | | | CACATACAAAGTTACCCACAGATTCCGTGGATAAGCA |
| | | | GGGGACTAACATGTGAGGCAAAACAGCAGGGCCGCGC |
| | | | CGGTGGCGTTTTTCCATAGGCTCCGCCCTCCTGCCAG |
| | | | AGTTCACATAAACAGACGCTTTTCCGGTGCATCTGTG |
| | | | GGAGCCGTGAGGCTCAACCATGAATCTGACAGTACGG |
| | | | GCGAAACCCGACAGGACTTAAAGATCCCCACCGTTTC |
| | | | CGGCtGGTCGCTCCCTCTTGCGCTCTCCTGTTCCGAC |
| | | | CCTGCCGTTTACCGGATACCTGTTCCGCCTTTCTCCC |
| | | | TTACGGGAAGTGTGGCGCTTTCTCATAGCTCACACAC |
| | | | TGGTATCTCGGCTCGGTGTAGGTCGTTCGCTCCAAGC |
| | | | TGGGCTGTAAGCAAGAACTCCCCGTTCAGCCCGACTG |
| | | | CTGCGCCTTATCCGGTAACTGTTCACTTGAGTCCAAC |
| | | | CCGGAAAAGCACGGTAAAACGCCACTGGCAGCAGCCA |
| | | | TTGGTAACTGGGAGTTCGCAGAGGATTTGTTTAGCTA |
| | | | AACACGCGGTTGCTCTTGAAGTGTGCGCCAAAGTCCG |
| | | | GCTACACTGGAAGGACAGATTTGGTTGCTGTGCTCTG |
| | | | CGAAAGCCAGTTACCACGGTTAAGCAGTTCCCCAACT |
| | | | GACTTAACCTTCGATCAAACCACCTCCCaatGTGGTT |
| | | | TTTTCGTTTACAGGGCAAAAGATTACGCGCAGAAAAA |
| | | | AAGGATCTCAAGAAGATCCTTTGATCTTTTCTACTGA |
| | | | ACCGCTCTAGATTTCAGTGCAATTTATCTCTTCAAAT |
| | | | GTAGCACCTGAAGTCAGCCCCATACGATATAAGTTGT |
| | | | AATTCTCATGTTAGTCATGCCCCGCGCCCACCGGAAG |
| | | | GAGCTGACTGGGTTGAAGGCTCTCAAGGGCATCGGTC |
| | | | GAGATCCCGGTGCCTAATGAGTGAGCTAACTTTTGAC |
| | | | gGCTAGCTCAGTCCTAGGGAtaATGCTAGCaccagcc |
| | | | TCGAgggaaaccacgtaagctccggcgtTTAAAcacc |
| | | | cataacagatacggactttctcaaaggagagttatca |
| | | | gTGAAAATCCGCCCGTTACATGACCGTGTCATCATCA |
| | | | AACGCTTGGAAGAAGAGCGTACCTCGGCGGGCGGGAT |
| | | | TGTCATTCCAGATAGCGCaGCTGAAAAACCGATGCGT |
| | | | GGTGAAATCCTGGCAGTGGGCAATGGAAAAGTGCTTG |
| | | | ATAATGGAGAGGTACGTGCTTTACAGGTGAAAGTGGG |
| | | | TGATAAAGTGCTCTTTGGGAAATACGCGGGTACGGAG |
| | | | GTTAAAGTAGATGGGGAAGATGTTGTTGTCATGCGTG |
| | | | AAGATGACATTCTGGCTGTGTTAGAATCTTAATCCGC |
| | | | GCACGACACTGAACATACGAATTTAAGGAATAAAGAT |
| | | | AATGGCGAAAGAAGTTGTGTATCGTGGTAGTGCGCGC |
| | | | CAGCGTATGATGCAGGGTATTGAAATTCTCGCTCGCG |
| | | | CCGCTATTCCAACGCTGGGGGCAACCGGCCCGAGCGT |
| | | | CATGATTCAACATCGCGCCGATGGTCTGCCACCCATT |
| | | | TCTACACGCGATGGCGTTACCGTAGCGAATTCTATTG |
| | | | TTTTAAAAGACCGTGTCGCGAACCTGGGTGCCCGCCT |
| | | | GCTGCGCGACGTAGCCGGTACAATGAGCCGTGAAGCC |
| | | | GGCGACGGCACGACGACTGCGATCGTATTGGCCCGCC |
| | | | ACATCGCCCGTGAGATGTTTAAATCGCTGGCCGTGGG |
| | | | TGCAGATCCGATCGCGCTGAAACGTGGTATCGATCGC |
| | | | GCCGTTGCTCGTGTGTCCGAAGATATTGGGGCGCGTG |
| | | | CGTGGCGTGGCGATAAAGAAAGCGTGATCCTGGGTGT |
| | | | CGCTGCTGTGGCGACGAAAGGCGAACCGGGCGTTGGC |
| | | | CGTCTGCTGCTGGAGGCTCTCGATGCAGTGGGTGTTC |
| | | | ACGGTGCCGTTTCTATCGAACTGGGCCAACGTCGTGA |
| | | | AGATCTGCTGGACGTCGTCGATGGCTATCGCTGGGAA |
| | | | AAAGGTTATTTATCTCCCTACTTTGTCACGGACCGTG |
| | | | CCCGCGAACTCGCGGAACTGGAGGATGTCTACCTGCT |
| | | | CATGACCGACCGCGAAGTGGTTGACTTCATCGACCTT |
| | | | GTACCTCTGCTGGAGGCCGTGACGGAAGCAGGAGGCT |
| | | | CCCTGCTGATTGCCGCGGATCGTGTGCACGAAAAGGC |
| | | | CTTAGCGGGGCTGCTTCTGAATCACGTGCGCGGTGTC |
| | | | TTCAAGGCCGTGGCCGTAACCGCTCCGGGTTTTGGCG |
| | | | ACAAACGCCCGAACCGTTTACTTGACCTGGCCGCGTT |
| | | | AACCGGCGGTCGTGCCGTGCTCGAAGCTCAAGGCGAC |
| | | | CGTCTGGACCGTGTTACCCTCGCGGATCTGGGCCGTG |
| | | | TGCGCCGTGCCGTGGTGTCGGCAGATGATACCGCGCT |
| | | | GCTTGGCATCCCGGGCACCGAAGCTAGCCGTGCACGC |
| | | | CTCGAAGGTCTGCGTTTAGAAGCAGAgCAGTACCGTG |
| | | | CGCTGAAACCAGGGCAGGGTTCTGCCACCGGGCGCCT |
| | | | GCACGAACTTGAAGAAATTGAAGCGCGCATTGTGGGT |
| | | | CTgTCCGGAAAGAGCGCCGTTTATCGCGTCGGAGGTG |
| | | | TGACCGATGTGGAAATGAAAGAGCGCATGGTTCGCAT |
| | | | CGAAggaGCTTACCGTTCGGTGGTAAGTGCGCTGGAG |
| | | | GAAGGCGTGCTCCCTGGCGGTGGTGTCGGCTTTCTGG |
| | | | GTAGTATGCCGGTGCTTGCGGAATTGGAGGCCCGCGA |
| | | | CGCAGATGAAGCTCGCGGGATTGGGATTGTACGCAGC |
| | | | GCCTTAACGGAGCCTCTTCGTATTATCGGCGAAAATA |
| | | | GTGGCTTGAGCGGTGAAGCCGTTGTTGCCAAAGTCAT |
| | | | GGATCATGCCAACCCGGGATGGGGTTACGACCAGGAG |
| | | | TCTGGCTCTTTTTGCGACCTGCATGCGCGTGGGATCT |
| | | | GGGATGCTGCTAAAGTGTTACGTCTCGCGTTGGAGAA |
| | | | GGCAGCCTCTGTTGCTGGGACCTTTCTGACAACCGAA |
| | | | GCTGTTGTTCTCGAAATTCCGGATACAGATGCGTTCG |
| | | | CAGGGTTCAGTGCAGAATGGGCTGCCGCCACGCGCGA |
| | | | AGATCCGCGCGTATGAgtttaaacgcggccgcAATTT |
| | | | GAACGcacccataacagatacggactttctcaaagga |
| | | | gagttatcaATGAATATTCGTCCATTGCATGATCGCG |
| | | | TGATCGTCAAGCGTAAAGAAGTTGAAACTAAATCTGC |
| | | | TGGCGGCATCGTTCTGACCGGCTCTGCAGCGGCTAAA |
| | | | TCCACCCGCGGCGAAGTGCTGGCTGTCGGCAATGGCC |
| | | | GTATCCTTGAAAATGGCGAAGTGAAGCCGCTGGATGT |
| | | | GAAAGTTGGCGACATCGTTATTTTCAACGATGGCTAC |
| | | | GGTGTGAAATCTGAGAAGATCGACAATGAAGAAGTGT |
| | | | TGATCATGTCCGAAAGCGACATTCTGGCAATTGTTGA |
| | | | AGCGTAATCCGCGCACGACACTGAACATACGAATTTA |
| | | | AGGAATAAAGATAatgGCAGCTAAAGACGTAAAATTC |
| | | | GGTAACGACGCTCGTGTGAAAATGCTGCGCGGCGTAA |
| | | | ACGTACTGGCAGATGCAGTGAAAGTTACCCTCGGTCC |
| | | | AAAAGGCCGTAACGTAGTTCTGGATAAATCTTTCGGT |
| | | | GCACCGACCATCACCAAAGATGGTGTTTCCGTTGCTC |
| | | | GTGAAATCGAACTGGAAGACAAGTTCGAAAATATGGG |
| | | | TGCGCAGATGGTGAAAGAAGTTGCCTCTAAAGCAAAC |
| | | | GACGCTGCAGGCGACGGTACCACCACTGCAACCGTAC |
| | | | TGGCTCAGGCTATCATCACTGAAGGTCTGAAAGCTGT |
| | | | TGCTGCGGGCATGAACCCGATGGACCTGAAACGTGGT |
| | | | ATCGACAAAGCGGTTACCGCTGCAGTTGAAGAACTGA |
| | | | AAGCGCTGTCCGTACCATGCTCTGACTCTAAAGCGAT |
| | | | TGCTCAGGTTGGTACCATCTCCGCTAACTCCGACGAA |
| | | | ACCGTAGGTAAACTGATCGCTGAAGCGATGGACAAAG |
| | | | TCGGTAAAGAAGGCGTTATCACCGTTGAAGACGGTAC |
| | | | CGGTCTGCAGGACGAACTGGACGTGGTTGAAGGTATG |
| | | | CAGTTCGACCGTGGCTACCTGTCTCCTTACTTCATCA |
| | | | ACAAGCCGGAAACTGGCGCAGTAGAACTGGAAAGCCC |
| | | | GTTCATCCTGCTGGCTGACAAGAAAATCTCCAACATC |
| | | | CGCGAAATGCTGCCGGTTCTGGAAGCTGTTGCCAAAG |
| | | | CAGGCAAACCGCTGCTGATCATCGCTGAAGATGTAGA |
| | | | AGGCGAAGCGCTGGCAACTCTGGTTGTTAACACCATG |
| | | | CGTGGCATCGTGAAAGTCGCTGCGGTTAAAGCACCGG |
| | | | GCTTCGGCGATCGTCGTAAAGCTATGCTGCAGGATAT |
| | | | CGCAACCCTGACTGGCGGTACCGTGATCTCTGAAGAG |
| | | | ATCGGTATGGAGCTGGAAAAAGCAACCCTGGAAGACC |
| | | | TGGGTCAGGCTAAACGTGTTGTGATCAACAAAGACAC |
| | | | CACCACTATCATCGATGGCGTGGGTGAAGAAGCTGCA |
| | | | ATCCAGGGCCGTGTTGCTCAGATCCGTCAGCAGATTG |
| | | | AAGAAGCAACTTCTGACTACGACCGTGAAAAACTGCA |
| | | | GGAACGCGTAGCGAAACTGGCAGGCGGCGTTGCAGTT |
| | | | ATCAAAGTGGGTGCTGCTACCGAAGTTGAAATGAAAG |
| | | | AGAAAAAAGCACGCGTTGAAGATGCCCTGCACGCGAC |
| | | | CCGTGCTGCGGTAGAAGAAGGCGTGGTTGCTGGTGGT |
| | | | GGTGTTGCGCTGATCCGCGTAGCGTCTAAACTGGCTG |
| | | | ACCTGCGTGGTCAGAACGAAGACCAGAACGTGGGTAT |
| | | | CAAAGTTGCACTGCGTGCAATGGAAGCTCCGCTGCGT |
| | | | CAGATCGTATTGAACTGCGGCGAAGAACCGTCTGTTG |
| | | | TTGCTAACACCGTTAAAGGCGGCGACGGCAACTACGG |
| | | | TTACAACGCAGCAACCGAAGAATACGGCAACATGATC |
| | | | GACATGGGTATCCTGGATCCAACCAAAGTAACTCGTT |
| | | | CTGCTCTGCAGTACGCAGCTTCTGTGGCTGGCCTGAT |
| | | | GATCACCACCGAATGCATGGTTACCGACCTGCCGAAA |
| | | | AACGATGCAGCTGACTTAGGCGCTGCTGGCGGTATGG |
| | | | GCGGCATGATGtaagtttaaacgcggccgcAATTTGA |
| | | | ACGCCAGCACATGGACTCtcgaGTCTACTAGCGCAGC |
| | | | TTAATTAACCTAGGCTGCTGCCACCGCTGAGCAATAA |
| | | | CTAGCATAACCCCTTGGGGCCTCTAAACGGGTCTTGA |
| | | | GGGGTTTTTTGCTGAAACCTCAGGCATTTGAGAAGCA |
| | | | CACGGTCACACTGCTTCCGGTAGTCAATAAACCGGTA |
| | | | AACCAGCAATAGACATAAGCGGTGCATAATGTGCCTG |
| | | | TCAAATGGACGAAGCAGGGATTCTGCAAACCCTATGC |
| | | | TACTCCGTCAAGCCGTCAATTGTCTGATTCGTTACCA |
| | | | ATTATGACAACTTGACGGCTACATCATTCACTTTTTC |
| | | | TTCACAACCGGCACGGAACTCGCTCGGGCTGGCCCCG |
| | | | GTGCATTTTTTAAATACCCGCGAGAAATAGAGTTGAT |
| | | | CGTCAAAACCAACATTGCGACCGACGGTGGCGATAGG |
| | | | CATCCGGGTGGTGCTCAAAAGCAGCTTCGCCTGGCTG |
| | | | ATACGTTGGTCCTCGCGCCAGCTTAAGACGCTAATCC |
| | | | CTAACTGCTGGCGGAAAAGATGTGACAGACGCGACGG |
| | | | CGACAAGCAAACATGCTGTGCGACGCTGGCGATATCA |
| | | | AAATTGCTGTCTGCCAGGTGATCGCTGATGTACTGAC |
| | | | AAGCCTCGCGTACCCGATTATCCATCGGTGGATGGAG |
| | | | CGACTCGTTAATCGCTTCCATGCGCCGCAGTAACAAT |
| | | | TGCTCAAGCAGATTTATCGCCAGCAGCTCCGAATAGC |
| | | | GCCCTTCCCCTTGCCCGGCGTTAATGATTTGCCCAAA |
| | | | CAGGTCGCTGAAATGCGGCTGGTGCGCTTCATCCGGG |
| | | | CGAAAGAACCCCGTATTGGCAAATATTGACGGCCAGT |
| | | | TAAGCCATTCATGCCAGTAGGCGCGCGGACGAAAGTA |
| | | | AACCCACTGGTGATACCATTCGCGAGCCTCCGGATGA |
| | | | CGACCGTAGTGATGAATCTCTCCTGGCGGGAACAGCA |
| | | | AAATATCACCCGGTCGGCAAACAAATTCTCGTCCCTG |
| | | | ATTTTTCACCACCCCCTGACCGCGAATGGTGAGATTG |
| | | | AGAATATAACCTTTCATTCCCAGCGGTCGGTCGATAA |
| | | | AAAAATCGAGATAACCGTTGGCCTCAATCGGCGTTAA |
| | | | ACCCGCCACCAGATGGGCATTAAACGAGTATCCCGGC |
| | | | AGCAGGGGATCATTTTGCGCTTCAGCCATACTTTTCA |
| | | | TACTCCCGCCATTCAGAGAAGAAACCAATTGTCCATA |
| | | | TTGCATCAGACATTGCCGTCACTGCGTCTTTTACTGG |
| | | | CTCTTCTCGCTAACCAAACCGGTAACCCCGCTTATTA |
| | | | AAAGCATTCTGTAACAAAGCGGGACCAAAGCCATGAC |
| | | | AAAAACGCGTAACAAAAGTGTCTATAATCACGGCAGA |
| | | | AAAGTCCACATTGATTATTTGCACGGCGTCACACTTT |
| | | | GCTATGCCATAGCATTTTTATCCATAAGATTAGCGGA |
| | | | TCCTACCTGACGCTTTTTATCGCAACTCTggACaaTg |
| | | | TCTCCATACCCGTTTTTTTGGGcgacctcgtcggagg |
| | | | ttgtatgtccggtgttccgtgacgtcatcgggcattc |
| | | | atcattcatagaatgtgttacggaggaaacaagtaat |
| | | | ggcacttagcaccgcaaccaaggccgcgacggacgcg |
| | | | ctggctgccaatcgggcacccaccagcgGgaatgcac |
| | | | aggaagtgcaccgttggctccagagcttcaactggga |
| | | | tttcaagaacaaccggaccaagtacgccaccaagtac |
| | | | aagatggcgaacgagaccaaggaacagttcaagctga |
| | | | tcgccaaggaatatgcgcgcatggaggcagtcaagga |
| | | | cgaaaggcagttcggtagcctgcaggatgcgctgacc |
| | | | cgcctcaacgccggtgttcgcgttcatccgaagtgga |
| | | | acgagaccatgaaagtggtttcgaacttcctggaagt |
| | | | gggcgaatacaacgccatcgccgctaccgggatgctg |
| | | | tgggattccgcccaggcggcggaacagaagaacggct |
| | | | atctggcccaggtgttggatgaaatccgccacaccca |
| | | | ccagtgtgcctacgtcaactactacttcgcgaagaac |
| | | | ggccaggacccggccggtcacaacgatgctcgccgca |
| | | | cccgtaccatcggtccgctgtggaagggcatgaagcg |
| | | | cgtgttttccgacggcttcatttccggcgacgccgtg |
| | | | gaatgctccctcaacctgcagctggtgggtgaggcct |
| | | | gcttcaccaatccgctgatcgtcgcagtgaccgaatg |
| | | | ggctgccgccaacggcgatgaaatcaccccgacggtg |
| | | | ttcctgtcgatcgagaccgacgaactgcgccacatgg |
| | | | ccaacggttaccagaccgtcgtttccatcgccaacga |
| | | | tccggcttccgccaagtatctcaacacggacctgaac |
| | | | aacgccttctggacccagcagaagtacttcacgccgg |
| | | | tgttgggcatgctgttcgagtatggctccaagttcaa |
| | | | ggtcgagccgtgggtcaagacgtggaaccgctgggtg |
| | | | tacgaggactggggcggcatctggatcggccgtctgg |
| | | | gcaagtacggggtggagtcgccgcgcagcctcaagga |
| | | | cgccaagcaggacgcttactgggctcaccacgacctg |
| | | | tatctgctggcttatgcgctgtggccgGGcggcttct |
| | | | tccgtctggcgctgccggatcaggaagaaatggagtg |
| | | | gttcgaggccaactaccccggctggtacgaccactac |
| | | | ggcaagatctacgaggaatggcgcgcccgcggttgcg |
| | | | aggatccgtcctcgggcttcatcccgctgatgtggtt |
| | | | catcgaaaacaaccatcccatctacatcgatcgcgtg |
| | | | tcgcaagtgccgttctgcccgagcttggccaagggcg |
| | | | ccagcaccctgcgcgtgcacgagtacaacggccagat |
| | | | gcacaccttcagcgaccagtggggcgagcgcatgtgg |
| | | | ctggccgagccggagcgctacgagtgccagaacatct |
| | | | tcgaacagtacgaaggacgcgaactgtcggaagtgat |
| | | | cgccgaactgcacgggctgcgcagtgatggcaagacc |
| | | | ctgatcgcccagccgcatgtccgtggcgacaagctgt |
| | | | ggacgttggacgatatcaaacgcctgaactgcgtctt |
| | | | caagaacccggtgaaggcattcaattgaaacgggtgt |
| | | | cgggctccgtcacagggcggggcccgacgcacgatcg |
| | | | ttcgatcaacctcaaaccaaaaaggaacatcgatatg |
| | | | agcatgttaggagaaagacgccgcggtctgaccgatc |
| | | | cggaaatggcggccgtcattttgaaggcgcttcctga |
| | | | agctccgctggacggcaacaacaagatgggttatttc |
| | | | gtcaccccccgctggaaacgcttgacggaatatgaag |
| | | | ccctgaccgtttatgcgcagcccaacgccgactggat |
| | | | cgccggcggcctggactggggcgactggacccagaaa |
| | | | ttccacggcggccgcccttcctggggcaacgagacca |
| | | | cggagctgcgcaccgtcgactggttcaagcaccgtga |
| | | | cccgctccgccgttggcatgcgccgtacgtcaaggac |
| | | | aaggccgaggaatggcgctacaccgaccgcttcctgc |
| | | | agggttactccgccgacggtcagatccgggcgatgaa |
| | | | cccgacctggcgggacgagttcatcaaccggtattgg |
| | | | ggcgccttcctgttcaacgaatacggattgttcaacg |
| | | | ctcattcgcagggcgcccgggaggcgctgtcggacgt |
| | | | aacccgcgtcagcctggctttctggggcttcgacaag |
| | | | atcgacatcgcccagatgatccaactcgaacggggtt |
| | | | tcctcgccaagatcgtacccggtttcgacgagtccac |
| | | | agcggtgccgaaggccgaatggacgaacggggaggtc |
| | | | tacaagagcgcccgtctggccgtggaagggctgtggc |
| | | | aggaggtgttcgactggaacgagagcgctttctcggt |
| | | | gcacgccgtctatgacgcgctgttcggtcagttcgtc |
| | | | cgccgcgagttctttcagcggctggctccccgcttcg |
| | | | gcgacaatctgacgccattcttcatcaaccaggccca |
| | | | gacatacttccagatcgccaagcagggcgtacaggat |
| | | | ctgtattacaactgtctgggtgacgatccggagttca |
| | | | gcgattacaaccgtaccgtgatgcgcaactggaccgg |
| | | | caagtggctggagcccacgatcgccgctctgcgcgac |
| | | | ttcatggggctgtttgcgaagctgccggcgggcacca |
| | | | ctgacaaggaagaaatcaccgcgtccctgtaccgggt |
| | | | ggtcgacgactggatcgaggactacgccagcaggatc |
| | | | gacttcaaggcggaccgcgatcagatcgttaaagcgg |
| | | | ttctggcaggattgaaataatagaggaactattacga |
| | | | tgagcgtaaacagcaacgcatacgacgccggcatcat |
| | | | gggcctgaaaggcaaggacttcgccgatcagttcttt |
| | | | gccgacgaaaaccaagtggtccatgaaagcgacacgg |
| | | | tcgttctggtcctcaagaagtcggacgagatcaatac |
| | | | ctttatcgaggagatccttctgacggactacaagaag |
| | | | aacgtcaatccgacggtaaacgtggaagaccgcgcgg |
| | | | gttactggtggatcaaggccaacggcaagatcgaggt |
| | | | cgattgcgacgagatttccgagctgttggggcggcag |
| | | | ttcaacgtctacgacttcctcgtcgacgtttcctcca |
| | | | ccatcggccgggcctataccctgggcaacaagttcac |
| | | | cattaccagtgagctgatgggcctggaccgcaagctc |
| | | | gaagactCtcacgcttaaggagaatgacatggcgaaa |
| | | | ctgggtatacacagcaacgacacccgcgacgcctggg |
| | | | tgaacaagatcgcgcagctcaacaccctggaaaaagc |
| | | | ggccgagatgctgaagcagttccggatggaccacacc |
| | | | acgccgttccgcaacagctacgaactggacaacgact |
| | | | acctctggatcgaggccaagctcgaagagaaggtcgc |
| | | | cgtcctcaaggcaGAAgccttcaacgaggtggacttc |
| | | | cgtcataagaccgctttcggcgaggatgccaagtccg |
| | | | ttctggacggcaccgtcgcgaagatgaacgcggccaa |
| | | | ggacaagtgggaggcggagaagatccatatcggtttc |
| | | | cgccaggcctacaagccgccgatcatgccggtgaact |
| | | | atttcctggacggcgagcgTcagttggggacccggct |
| | | | gatggaactgcgcaacctcaactactacgacacgccg |
| | | | ctggaagaactgcgcaaacagcgcggtgtgcgggtgg |
| | | | tgcatctgcagtcgccgcactgaagggaggaagtctc |
| | | | gccctggacgcgacggcatcgccgtgaagtccagggg |
| | | | gcagggatgccgttccgggTcggcaggctggcccgga |
| | | | atctctggttttcagggggcgtgccggtccacggctc |
| | | | ccccctccatctttcgtaaggaaatcaccatggtcga |
| | | | atcggcatttcagccattttcgggcgacgcagacgaa |
| | | | tggttcgaggaaccacggccccaggccggtttcttcc |
| | | | cttccgcggactggcatctgctcaaacgggacgagac |
| | | | ctacgcagcctatgccaaggatctcgatttcatgtgg |
| | | | cggtgggtcatcgtccgggaagaaaggatcgtccagg |
| | | | agggttgctcgatcagcctggagtcgtcgatccgcgc |
| | | | cgtgacgcacgtactgaattattttggtatgaccgaa |
| | | | caacgcgccccggcagaggaccggaccggcggagttc |
| | | | aacattgaacaggtaagtttatgcagcgagttcacac |
| | | | tatcacggcggtgacggaggatggcgaatcgctccgc |
| | | | ttcgaatgccgttcggacgaggacgtcatcaccgccg |
| | | | ccctgcgccagaacatctttctgatgtcgtcctgccg |
| | | | ggagggcggctgtgcgacctgcaaggccttgtgcagc |
| | | | gaaggggactacgacctcaagggctgcagcgttcagg |
| | | | cgctgccgccggaagaggaggaggaagggttggtgtt |
| | | | gttgtgccggacctacccgaagaccgacctggaaatc |
| | | | gaactgccctatacccattgccgcatcagttttggtg |
| | | | aggtcggcagtttcgaggcggaggtcgtcggcctcaa |
| | | | ctgggtttcgagcaacaccgtccagtttcttttgcag |
| | | | aagcggcccgacgagtgcggcaaccgtggcgtgaaat |
| | | | tcgaacccggtcagttcatggacctgaccatccccgg |
| | | | caccgatgtctcccgctcctactcgccggcgaacctt |
| | | | cctaatcccgaaggccgcctggagttcctgatccgcg |
| | | | tgttaccggagggacggttttcggactacctgcgcaa |
| | | | tgacgcgcgtgtcggacaggtcctctcggtcaaaggg |
| | | | ccactgggcgtgttcggtctcaaggagcggggcatgg |
| | | | cgccgcgctatttcgtggccggcggcaccgggttggc |
| | | | gccggtggtctcgatggtgcggcagatgcaggagtgg |
| | | | accgcgccgaacgagacccgcatctatttcggtgtga |
| | | | acaccgagccggaattgttctacatcgacgagctcaa |
| | | | atccctggaacgatcgatgcgcaatctcaccgtgaag |
| | | | gcctgtgtctggcacccgagcggggactgggaaggcg |
| | | | agcagggctcgcccatcgatgcgttgcgggaagacct |
| | | | ggagtcctccgacgccaacccggacatttatttgtgc |
| | | | ggtccgccgggcatgatcgatgccgcctgcgagctgg |
| | | | tacgcagccgcggtatccccggcgaacaggtcttctt |
| | | | cgaaaaattcctgccgtccggggcggcctgaaccggg |
| | | | gaagtaccgtgaccaccgagcagttcccgccccaatt |
| | | | cctgcgtgaaatgatcgagcagctggacgccagcatc |
| | | | caggagctcgcacgcaaggaaaagggacttgcggcat |
| | | | ccctgggcacgggccgggtcgccgagctcaaggaata |
| | | | ctgggaccacgttGTTACAACCAATTAACCAATTCTG |
| | | | ACTATTTAACGACCCTGCCCTGAACCGACGACCGGGT |
| | | | CATCGTGGCCGGATCTTGCGGCCCCTCGGCTTGAACG |
| | | | AATTGTTAGACATTATTTGCCGACTACCTTGGTGATC |
| | | | TCGCCTTTCACGTAGTGGACAAATTCTTCCAACTGAT |
| | | | CTGCGCGCGAGGCCAAGCGATCTTCTTCTTGTCCAAG |
| | | | ATAAGCCTGTCTAGCTTCAAGTATGACGGGCTGATAC |
| | | | TGGGCCGGCAGGCGCTCCATTGCCCAGTCGGCAGCGA |
| | | | CATCCTTCGGCGCGATTTTGCCGGTTACTGCGCTGTA |
| | | | CCAAATGCGGGACAACGTAAGCACTACATTTCGCTCA |
| | | | TCGCCAGCCCAGTCGGGCGGCGAGTTCCATAGCGTTA |
| | | | AGGTTTCATTTAGCGCCTCAAATAGATCCTGTTCAGG |
| | | | AACCGGATCAAAGAGTTCCTCCGCCGCTGGACCTACC |
| | | | AAGGCAACGCTATGTTCTCTTGCTTTTGTCAGCAAGA |
| | | | TAGCCAGATCAATGTCGATCGTGGCTGGCTCGAAGAT |
| | | | ACCTGCAAGAATGTCATTGCGCTGCCATTCTCCAAAT |
| | | | TGCAGTTCGCGCTTAGCTGGATAACGCCACGGAATGA |
| | | | TGTCGTCGTGCACAACAATGGTGACTTCTACAGCGCG |
| | | | GAGAATCTCGCTCTCTCCAGGGGAAGCCGAAGTTTCC |
| | | | AAAAGGTCGTTGATCAAAGCTCGCCGCG |
| 45. | pYZ40 | | CGGCGGGCGCTGCGGACACATACAAAGTTACCCACAG |
| | | | ATTCCGTGGATAAGCAGGGGACTAACATGTGAGGCAA |
| | | | AACAGCAGGGCCGCGCCGGTGGCGTTTTTCCATAGGC |
| | | | TCCGCCCTCCTGCCAGAGTTCACATAAACAGACGCTT |
| | | | TTCCGGTGCATCTGTGGGAGCCGTGAGGCTCAACCAT |
| | | | GAATCTGACAGTACGGGCGAAACCCGACAGGACTTAA |
| | | | AGATCCCCACCGTTTCCGGCTGGTCGCTCCCTCTTGC |
| | | | GCTCTCCTGTTCCGACCCTGCCGTTTACCGGATACCT |
| | | | GTTCCGCCTTTCTCCCTTACGGGAAGTGTGGCGCTTT |
| | | | CTCATAGCTCACACACTGGTATCTCGGCTCGGTGTAG |
| | | | GTCGTTCGCTCCAAGCTGGGCTGTAAGCAAGAACTCC |
| | | | CCGTTCAGCCCGACTGCTGCGCCTTATCCGGTAACTG |
| | | | TTCACTTGAGTCCAACCCGGAAAAGCACGGTAAAACG |
| | | | CCACTGGCAGCAGCCATTGGTAACTGGGAGTTCGCAG |
| | | | AGGATTTGTTTAGCTAAACACGCGGTTGCTCTTGAAG |
| | | | TGTGCGCCAAAGTCCGGCTACACTGGAAGGACAGATT |
| | | | TGGTTGCTGTGCTCTGCGAAAGCCAGTTACCACGGTT |
| | | | AAGCAGTTCCCCAACTGACTTAACCTTCGATCAAACC |
| | | | ACCTCCCAATGTGGTTTTTTCGTTTACAGGGCAAAAG |
| | | | ATTACGCGCAGAAAAAAAGGATCTCAAGAAGATCCTT |
| | | | TGATCTTTTCTACTGAACCGCTCTAGATTTCAGTGCA |
| | | | ATTTATCTCTTCAAATGTAGCACCTGAAGTCAGCCCC |
| | | | ATACGATATAAGTTGTAATTCTCATGTTAGTCATGCC |
| | | | CCGCGCCCACCGGAAGGAGCTGACTGGGTTGAAGGCT |
| | | | CTCAAGGGCATCGGTCGAGATCCCGGTGCCTAATGAG |
| | | | TGAGCTAACTTTTGACGGCTAGCTCAGTCCTAGGGAT |
| | | | AATGCTAGCACCAGCCTCGAGGGAAACCACGTAAGCT |
| | | | CCGGCGTTTAAACACCCATAACAGATACGGACTTTCT |
| | | | CAAAGGAGAGTTATCAGTGAAAATCCGCCCGTTACAT |
| | | | GACCGTGTCATCATCAAACGCTTGGAAGAAGAGCGTA |
| | | | CCTCGGCGGGCGGGATTGTCATTCCAGATAGCGCAGC |
| | | | TGAAAAACCGATGCGTGGTGAAATCCTGGCAGTGGGC |
| | | | AATGGAAAAGTGCTTGATAATGGAGAGGTACGTGCTT |
| | | | TACAGGTGAAAGTGGGTGATAAAGTGCTCTTTGGGAA |
| | | | ATACGCGGGTACGGAGGTTAAAGTAGATGGGGAAGAT |
| | | | GTTGTTGTCATGCGTGAAGATGACATTCTGGCTGTGT |
| | | | TAGAATCTTAATCCGCGCACGACACTGAACATACGAA |
| | | | TTTAAGGAATAAAGATAATGGCGAAAGAAGTTGTGTA |
| | | | TCGTGGTAGTGCGCGCCAGCGTATGATGCAGGGTATT |
| | | | GAAATTCTCGCTCGCGCCGCTATTCCAACGCTGGGGG |
| | | | CAACCGGCCCGAGCGTCATGATTCAACATCGCGCCGA |
| | | | TGGTCTGCCACCCATTTCTACACGCGATGGCGTTACC |
| | | | GTAGCGAATTCTATTGTTTTAAAAGACCGTGTCGCGA |
| | | | ACCTGGGTGCCCGCCTGCTGCGCGACGTAGCCGGTAC |
| | | | AATGAGCCGTGAAGCCGGCGACGGCACGACGACTGCG |
| | | | ATCGTATTGGCCCGCCACATCGCCCGTGAGATGTTTA |
| | | | AATCGCTGGCCGTGGGTGCAGATCCGATCGCGCTGAA |
| | | | ACGTGGTATCGATCGCGCCGTTGCTCGTGTGTCCGAA |
| | | | GATATTGGGGCGCGTGCGTGGCGTGGCGATAAAGAAA |
| | | | GCGTGATCCTGGGTGTCGCTGCTGTGGCGACGAAAGG |
| | | | CGAACCGGGCGTTGGCCGTCTGCTGCTGGAGGCTCTC |
| | | | GATGCAGTGGGTGTTCACGGTGCCGTTTCTATCGAAC |
| | | | TGGGCCAACGTCGTGAAGATCTGCTGGACGTCGTCGA |
| | | | TGGCTATCGCTGGGAAAAAGGTTATTTATCTCCCTAC |
| | | | TTTGTCACGGACCGTGCCCGCGAACTCGCGGAACTGG |
| | | | AGGATGTCTACCTGCTCATGACCGACCGCGAAGTGGT |
| | | | TGACTTCATCGACCTTGTACCTCTGCTGGAGGCCGTG |
| | | | ACGGAAGCAGGAGGCTCCCTGCTGATTGCCGCGGATC |
| | | | GTGTGCACGAAAAGGCCTTAGCGGGGCTGCTTCTGAA |
| | | | TCACGTGCGCGGTGTCTTCAAGGCCGTGGCCGTAACC |
| | | | GCTCCGGGTTTTGGCGACAAACGCCCGAACCGTTTAC |
| | | | TTGACCTGGCCGCGTTAACCGGCGGTCGTGCCGTGCT |
| | | | CGAAGCTCAAGGCGACCGTCTGGACCGTGTTACCCTC |
| | | | GCGGATCTGGGCCGTGTGCGCCGTGCCGTGGTGTCGG |
| | | | CAGATGATACCGCGCTGCTTGGCATCCCGGGCACCGA |
| | | | AGCTAGCCGTGCACGCCTCGAAGGTCTGCGTTTAGAA |
| | | | GCAGAGCAGTACCGTGCGCTGAAACCAGGGCAGGGTT |
| | | | CTGCCACCGGGCGCCTGCACGAACTTGAAGAAATTGA |
| | | | AGCGCGCATTGTGGGTCTGTCCGGAAAGAGCGCCGTT |
| | | | TATCGCGTCGGAGGTGTGACCGATGTGGAAATGAAAG |
| | | | AGCGCATGGTTCGCATCGAAGGAGCTTACCGTTCGGT |
| | | | GGTAAGTGCGCTGGAGGAAGGCGTGCTCCCTGGCGGT |
| | | | GGTGTCGGCTTTCTGGGTAGTATGCCGGTGCTTGCGG |
| | | | AATTGGAGGCCCGCGACGCAGATGAAGCTCGCGGGAT |
| | | | TGGGATTGTACGCAGCGCCTTAACGGAGCCTCTTCGT |
| | | | ATTATCGGCGAAAATAGTGGCTTGAGCGGTGAAGCCG |
| | | | TTGTTGCCAAAGTCATGGATCATGCCAACCCGGGATG |
| | | | GGGTTACGACCAGGAGTCTGGCTCTTTTTGCGACCTG |
| | | | CATGCGCGTGGGATCTGGGATGCTGCTAAAGTGTTAC |
| | | | GTCTCGCGTTGGAGAAGGCAGCCTCTGTTGCTGGGAC |
| | | | CTTTCTGACAACCGAAGCTGTTGTTCTCGAAATTCCG |
| | | | GATACAGATGCGTTCGCAGGGTTCAGTGCAGAATGGG |
| | | | CTGCCGCCACGCGCGAAGATCCGCGCGTATGAGTTTA |
| | | | AACGCGGCCGCAATTTGAACGCACCCATAACAGATAC |
| | | | GGACTTTCTCAAAGGAGAGTTATCAATGAATATTCGT |
| | | | CCATTGCATGATCGCGTGATCGTCAAGCGTAAAGAAG |
| | | | TTGAAACTAAATCTGCTGGCGGCATCGTTCTGACCGG |
| | | | CTCTGCAGCGGCTAAATCCACCCGCGGCGAAGTGCTG |
| | | | GCTGTCGGCAATGGCCGTATCCTTGAAAATGGCGAAG |
| | | | TGAAGCCGCTGGATGTGAAAGTTGGCGACATCGTTAT |
| | | | TTTCAACGATGGCTACGGTGTGAAATCTGAGAAGATC |
| | | | GACAATGAAGAAGTGTTGATCATGTCCGAAAGCGACA |
| | | | TTCTGGCAATTGTTGAAGCGTAATCCGCGCACGACAC |
| | | | TGAACATACGAATTTAAGGAATAAAGATAATGGCAGC |
| | | | TAAAGACGTAAAATTCGGTAACGACGCTCGTGTGAAA |
| | | | ATGCTGCGCGGCGTAAACGTACTGGCAGATGCAGTGA |
| | | | AAGTTACCCTCGGTCCAAAAGGCCGTAACGTAGTTCT |
| | | | GGATAAATCTTTCGGTGCACCGACCATCACCAAAGAT |
| | | | GGTGTTTCCGTTGCTCGTGAAATCGAACTGGAAGACA |
| | | | AGTTCGAAAATATGGGTGCGCAGATGGTGAAAGAAGT |
| | | | TGCCTCTAAAGCAAACGACGCTGCAGGCGACGGTACC |
| | | | ACCACTGCAACCGTACTGGCTCAGGCTATCATCACTG |
| | | | AAGGTCTGAAAGCTGTTGCTGCGGGCATGAACCCGAT |
| | | | GGACCTGAAACGTGGTATCGACAAAGCGGTTACCGCT |
| | | | GCAGTTGAAGAACTGAAAGCGCTGTCCGTACCATGCT |
| | | | CTGACTCTAAAGCGATTGCTCAGGTTGGTACCATCTC |
| | | | CGCTAACTCCGACGAAACCGTAGGTAAACTGATCGCT |
| | | | GAAGCGATGGACAAAGTCGGTAAAGAAGGCGTTATCA |
| | | | CCGTTGAAGACGGTACCGGTCTGCAGGACGAACTGGA |
| | | | CGTGGTTGAAGGTATGCAGTTCGACCGTGGCTACCTG |
| | | | TCTCCTTACTTCATCAACAAGCCGGAAACTGGCGCAG |
| | | | TAGAACTGGAAAGCCCGTTCATCCTGCTGGCTGACAA |
| | | | GAAAATCTCCAACATCCGCGAAATGCTGCCGGTTCTG |
| | | | GAAGCTGTTGCCAAAGCAGGCAAACCGCTGCTGATCA |
| | | | TCGCTGAAGATGTAGAAGGCGAAGCGCTGGCAACTCT |
| | | | GGTTGTTAACACCATGCGTGGCATCGTGAAAGTCGCT |
| | | | GCGGTTAAAGCACCGGGCTTCGGCGATCGTCGTAAAG |
| | | | CTATGCTGCAGGATATCGCAACCCTGACTGGCGGTAC |
| | | | CGTGATCTCTGAAGAGATCGGTATGGAGCTGGAAAAA |
| | | | GCAACCCTGGAAGACCTGGGTCAGGCTAAACGTGTTG |
| | | | TGATCAACAAAGACACCACCACTATCATCGATGGCGT |
| | | | GGGTGAAGAAGCTGCAATCCAGGGCCGTGTTGCTCAG |
| | | | ATCCGTCAGCAGATTGAAGAAGCAACTTCTGACTACG |
| | | | ACCGTGAAAAACTGCAGGAACGCGTAGCGAAACTGGC |
| | | | AGGCGGCGTTGCAGTTATCAAAGTGGGTGCTGCTACC |
| | | | GAAGTTGAAATGAAAGAGAAAAAAGCACGCGTTGAAG |
| | | | ATGCCCTGCACGCGACCCGTGCTGCGGTAGAAGAAGG |
| | | | CGTGGTTGCTGGTGGTGGTGTTGCGCTGATCCGCGTA |
| | | | GCGTCTAAACTGGCTGACCTGCGTGGTCAGAACGAAG |
| | | | ACCAGAACGTGGGTATCAAAGTTGCACTGCGTGCAAT |
| | | | GGAAGCTCCGCTGCGTCAGATCGTATTGAACTGCGGC |
| | | | GAAGAACCGTCTGTTGTTGCTAACACCGTTAAAGGCG |
| | | | GCGACGGCAACTACGGTTACAACGCAGCAACCGAAGA |
| | | | ATACGGCAACATGATCGACATGGGTATCCTGGATCCA |
| | | | ACCAAAGTAACTCGTTCTGCTCTGCAGTACGCAGCTT |
| | | | CTGTGGCTGGCCTGATGATCACCACCGAATGCATGGT |
| | | | TACCGACCTGCCGAAAAACGATGCAGCTGACTTAGGC |
| | | | GCTGCTGGCGGTATGGGCGGCATGATGTAAGTTTAAA |
| | | | CGCGGCCGCAATTTGAACGCCAGCACATGGACTCTCG |
| | | | AGTCTACTAGCGCAGCTTAATTAACCTAGGCTGCTGC |
| | | | CACCGCTGAGCAATAACTAGCATAACCCCTTGGGGCC |
| | | | TCTAAACGGGTCTTGAGGGGTTTTTTGCTGAAACCTC |
| | | | AGGCATTTGAGAAGCACACGGTCACACTGCTTCCGGT |
| | | | AGTCAATAAACCAGCTCAAGGAATACTGGGACCACGT |
| | | | TGTTACAACCAATTAACCAATTCTGACTATTTAACGA |
| | | | CCCTGCCCTGAACCGACGACCGGGTCATCGTGGCCGG |
| | | | ATCTTGCGGCCCCTCGGCTTGAACGAATTGTTAGACA |
| | | | TTATTTGCCGACTACCTTGGTGATCTCGCCTTTCACG |
| | | | TAGTGGACAAATTCTTCCAACTGATCTGCGCGCGAGG |
| | | | CCAAGCGATCTTCTTCTTGTCCAAGATAAGCCTGTCT |
| | | | AGCTTCAAGTATGACGGGCTGATACTGGGCCGGCAGG |
| | | | CGCTCCATTGCCCAGTCGGCAGCGACATCCTTCGGCG |
| | | | CGATTTTGCCGGTTACTGCGCTGTACCAAATGCGGGA |
| | | | CAACGTAAGCACTACATTTCGCTCATCGCCAGCCCAG |
| | | | TCGGGCGGCGAGTTCCATAGCGTTAAGGTTTCATTTA |
| | | | GCGCCTCAAATAGATCCTGTTCAGGAACCGGATCAAA |
| | | | GAGTTCCTCCGCCGCTGGACCTACCAAGGCAACGCTA |
| | | | TGTTCTCTTGCTTTTGTCAGCAAGATAGCCAGATCAA |
| | | | TGTCGATCGTGGCTGGCTCGAAGATACCTGCAAGAAT |
| | | | GTCATTGCGCTGCCATTCTCCAAATTGCAGTTCGCGC |
| | | | TTAGCTGGATAACGCCACGGAATGATGTCGTCGTGCA |
| | | | CAACAATGGTGACTTCTACAGCGCGGAGAATCTCGCT |
| | | | CTCTCCAGGGGAAGCCGAAGTTTCCAAAAGGTCGTTG |
| | | | ATCAAAGCTCGCCGCGTTGTTTCATCAAGCCTTACGG |
| | | | TCACCGTAACCAGCAAATCAATATCACTGTGTGGCTT |
| | | | CAGGCCGCCATCCACTGCGGAGCCGTACAAATGTACG |
| | | | GCCAGCAACGTCGGTTCGAGATGGCGCTCGATGACGC |
| | | | CAACTACCTCTGATAGTTGAGTCGATACTTCGGCGAT |
| | | | CACCGCTTCCCTCATACTCTTCCTTTTTCAATATTAT |
| | | | TGAAGCATTTATCAGGGTTATTGTCTCATGAGCGGAT |
| | | | ACATATTTGAATGTATTTAGAAAAATAAACAAATAGC |
| | | | TAGCTCACTCGGTCGCTACGCTCCGGGCGTGAGACTG |
| 46. | pNH296 | | CCGACACCATCGAATGGTGCAAAACCTTTCGCGGTAT |
| | | | GGCATGATAGCGCCCGGAAGAGAGTCAATTCAGGGTG |
| | | | GTGAATGTGAAACCAGTAACGTTATACGATGTCGCAG |
| | | | AGTATGCCGGTGTCTCTTATCAGACCGTTTCCCGCGT |
| | | | GGTGAACCAGGCCAGCCACGTTTCTGCGAAAACGCGG |
| | | | GAAAAAGTGGAAGCGGCGATGGCGGAGCTGAATTACA |
| | | | TTCCCAACCGCGTGGCACAACAACTGGGGGCAAACA |
| | | | GTCGTTGCTGATTGGCGTTGCCACCTCCAGTCTGGCC |
| | | | CTGCACGCGCCGTCGCAAATTGTCGCGGCGATTAAAT |
| | | | CTCGCGCCGATCAACTGGGTGCCAGCGTGGTGGTGTC |
| | | | GATGGTAGAACGAAGCGGCGTCGAAGCCTGTAAAGCG |
| | | | GCGGTGCACAATCTTCTCGCGCAACGCGTCAGTGGGC |
| | | | TGATCATTAACTATCCGCTGGATGACCAGGATGCCAT |
| | | | TGCTGTGGAAGCTGCCTGCACTAATGTTCCGGCGTTA |
| | | | TTTCTTGATGTCTCTGACCAGACACCCATCAACAGTA |
| | | | TTATTTTCTCCCATGAAGACGGTACGCGACTGGGCGT |
| | | | GGAGCATCTGGTCGCATTGGGTCACCAGCAAATCGCG |
| | | | CTGTTAGCGGGCCCATTAAGTTCTGTCTCGGCGCGTC |
| | | | TGCGTCTGGCTGGCTGGCATAAATATCTCACTCGCAA |
| | | | TCAAATTCAGCCGATAGCGGAACGGGAAGGCGACTGG |
| | | | AGTGCCATGTCCGGTTTTCAACAAACCATGCAAATGC |
| | | | TGAATGAGGGCATCGTTCCCACTGCGATGCTGGTTGC |
| | | | CAACGATCAGATGGCGCTGGGCGCAATGCGCGCCATT |
| | | | ACCGAGTCCGGGCTGCGCGTTGGTGCGGATATTTCGG |
| | | | TAGTGGGATACGACGATACCGAAGACAGCTCATGTTA |
| | | | TATCCCGCCGTTAACCACCATCAAACAGGATTTTCGC |
| | | | CTGCTGGGGCAAACCAGCGTGGACCGCTTGCTGCAAC |
| | | | TCTCTCAGGGCCAGGCGGTGAAGGGCAATCAGCTGTT |
| | | | GCCCGTCTCACTGGTGAAAAGAAAAACCACCCTGGCG |
| | | | CCCAATACGCAAACCGCCTCTCCCCGCGCGTTGGCCG |
| | | | ATTCATTAATGCAGCTGGCACGACAGGTTTCCCGACT |
| | | | GGAAAGCGGGCAGTGAGCGCAACGCAATTAATGTAAG |
| | | | TTAGCTCACTCATTAGGCACAATTCTCATGTTTGACA |
| | | | GCTTATCATCGACTGCACGGTGCACCAATGCTTCTGG |
| | | | CGTCAGGCAGCCATCGGAAGCTGTGGTATGGCTGTGC |
| | | | AGGTCGTAAATCACTGCATAATTCGTGTCGCTCAAGG |
| | | | CGCACTCCCGTTCTGGATAATGTTTTTTGCGCCGACA |
| | | | TCATAACGGTTCTGGCAAATATTCTGAAATGAGCTGT |
| | | | TGACAATTAATCATCGGCTCGTATAATGTGTGGAATT |
| | | | GTGAGCGGATAACAATTTCACACAGGAAACAGCCAGT |
| | | | CCGTTTAGGTGTTTTCACGAGCAATTGACCAACAAGG |
| | | | ACGTCAATAGAGTTAAGGAGGGAGGGGATGACCACTG |
| | | | CTGCACCCCAAGAATTTACCGCTGCTGTTGTTGAAAA |
| | | | ATTCGGTCATGACGTGACCGTGAAGGATATTGACCTT |
| | | | CCAAAGCCAGGGCCACACCAGGCATTGGTGAAGGTAC |
| | | | TCACCTCCGGCATCTGCCACACCGACCTCCACGCCTT |
| | | | GGAGGGCGATTGGCCAGTAAAGCCGGAACCACCATTC |
| | | | GTACCAGGACACGAAGGTGTAGGTGAAGTTGTTGAGC |
| | | | TCGGACCAGGTGAACACGATGTGAAGGTCGGCGATAT |
| | | | TGTCGGCAATGCGTGGCTCTGGTCAGCGTGTGGCACC |
| | | | TGCGAATACTGCATCACCGGCAGGGAAACTCAGTGCA |
| | | | ACGAAGCTGAGTATGGTGGCTACACCCAAAATGGATC |
| | | | CTTCGGCCAGTACATGCTGGTGGATACCCGTTACGCC |
| | | | GCTCGCATCCCAGACGGCGTGGACTACCTCGAAGCAG |
| | | | CACCAATTCTGTGTGCAGGCGTGACTGTCTACAAGGC |
| | | | ACTCAAAGTCTCTGAAACCCGCCCGGGCCAATTCATG |
| | | | GTGATCTCCGGTGTCGGCGGACTTGGCCACATCGCAG |
| | | | TCCAATACGCAGCGGCGATGGGCATGCGTGTCATTGC |
| | | | GGTAGATATTGCCGATGACAAGCTGGAACTTGCCCGT |
| | | | AAGCACGGTGCGGAATTTACCGTGAATGCGCGTAATG |
| | | | AAGATTCAGGCGAAGCTGTACAGAAGTACACCAACGG |
| | | | TGGCGCACACGGCGTGCTTGTGACTGCAGTTCACGAG |
| | | | GCAGCATTCGGCCAGGCACTGGATATGGCTCGACGTG |
| | | | CAGGAACAATTGTGTTCAACGGTCTGCCACCGGGAGA |
| | | | GTTCCCAGCATCCGTGTTCAACATCGTATTCAAGGGC |
| | | | CTGACCATCCGTGGATCCCTCGTGGGAACCCGCCAAG |
| | | | ACTTGGCCGAAGCGCTCGATTTCTTTGCACGCGGACT |
| | | | AATCAAGCCAACCGTGAGTGAGTGCTCCCTCGATGAG |
| | | | GTCAATGGTGTGCTTGACCGCATGCGAAACGGCAAGA |
| | | | TCGATGGTCGTGTGGCGATTCGTTTCTAACATGCTAA |
| | | | GGTGCTGGCTGCATGCTAAGTTGATACGCCTGCGACA |
| | | | AATTTTTCTAGGAGCGTTAGTATGACTGTCTACGCAA |
| | | | ATCCAGGAACCGAAGGCTCGATCGTTAACTATGAAAA |
| | | | GCGCTACGAGAACTACATTGGTGGCAAGTGGGTTCCA |
| | | | CCGGTAGAGGGCCAGTACCTTGAGAACATTTCACCTG |
| | | | TCACTGGTGAAGTTTTCTGTGAGGTCGCACGTGGCAC |
| | | | CGCAGCGGACGTGGAGCTTGCACTGGATGCTGCACAT |
| | | | GCAGCCGCTGATGCGTGGGGCAAGACTTCTGTCGCTG |
| | | | AACGTGCTCTGATCCTGCACCGCATTGCGGACCGCAT |
| | | | GGAAGAGCACCTGGAAGAAATCGCAGTTGCAGAAACC |
| | | | TGGGAGAACGGCAAGGCAGTCCGTGAGACTCTTGCTG |
| | | | CAGATATCCCACTGGCAATCGACCACTTCCGCTACTT |
| | | | TGCTGGCGCGATCCGTGCTCAGGAAGATCGTTCCTCA |
| | | | CAGATCGACCACAACACTGTTGCTTACCACTTCAACG |
| | | | AGCCAATCGGTGTTGTTGGTCAGATCATTCCTTGGAA |
| | | | CTTCCCAATCCTCATGGCTACCTGGAAGCTCGCACCG |
| | | | GCACTTGCTGCAGGTAACGCGATCGTCATGAAGCCAG |
| | | | CTGAGCAGACCCCAGCATCCATTTTGTATCTGATTAA |
| | | | CATCATCGGCGATCTCATCCCAGAGGGCGTCCTCAAC |
| | | | ATCGTCAACGGACTCGGCGGTGAAGCAGGCGCTGCAC |
| | | | TGTCCGGCTCTAATCGGATTGGCAAGATTGCTTTCAC |
| | | | CGGTTCCACCGAGGTCGGCAAGCTGATCAACCGCGCT |
| | | | GCATCCGACAAGATCATTCCTGTCACCCTGGAGCTCG |
| | | | GCGGTAAGTCCCCATCCATCTTCTTCTCCGATGTTCT |
| | | | GTCACAGGATGACGCCTTCGCAGAGAAGGCAGTTGAA |
| | | | GGCTTCGCGATGTTCGCCCTCAATCAGGGTGAAGTTT |
| | | | GTACCTGTCCTTCCCGTGCACTTGTTCATGAGTCCAT |
| | | | CGCTGATGAATTCCTCGAGCTTGGCGTGAAGCGAGTT |
| | | | CAGAACATCAAGCTGGGTAACCCACTTGATACTGAAA |
| | | | CCATGATGGGTGCTCAGGCGTCCCAGGAGCAGATGGA |
| | | | CAAGATCTCCTCCTACCTGAAGATCGGCCCAGAAGAA |
| | | | GGCGCTCAAACCCTCACTGGTGGCAAGGTCAACAAGG |
| | | | TTGATGGCATGGAGAACGGTTACTACATTGAGCCAAC |
| | | | CGTTTTCCGCGGCACCAACGACATGAGGATCTTCCGC |
| | | | GAGGAAATCTTCGGACCAGTCCTTTCTGTTGCTACCT |
| | | | TCAGCGACTTCGATGAGGCCATCCGTATTGCAAACGA |
| | | | CACCAACTACGGCCTCGGCGCTGGTGTCTGGAGCCGT |
| | | | GACCAAAACACCATTTATCGTGCAGGTCGCGCAATCC |
| | | | AGGCTGGTCGAGTTTGGGTCAACCAGTACCACAACTA |
| | | | CCCAGCGCACTCCGCTTTCGGTGGATACAAGGAGTCC |
| | | | GGCATCGGCCGTGAGAACCACCTCATGATGCTGAACC |
| | | | ACTACCAGCAGACCAAGAACCTGTTGGTCTCCTACGA |
| | | | TCCAAACCCAACCGGACTGTTCTGAGTTTGTCGGTGA |
| | | | ACGCTCTCCTGAGTAGGACAAATCCGCCGGGAGCGGA |
| | | | TTTGAACGTTGCGAAGCAACGGCCCGGAGGGTGGCGG |
| | | | GCAGGACGCCCGCCATAAACTGCCAGGCATCAAATTA |
| | | | AGCAGAAGGCCATCCTGACGGATGGCCTTTTTGCGTT |
| | | | TCTACAAACTCTTTCGGTCCGTTGTTTATTTTTCTAA |
| | | | ATACATTCAAATATGTATCCGCTCATGAGACAATAAC |
| | | | CCTGATAAATGCTTCAATAATATTGAAAAAGGAAGAG |
| | | | TATGAGTATTCAACATTTCCGTGTCGCCCTTATTCCC |
| | | | TTTTTTGCGGCATTTTGCCTTCCTGTTTTTGCTCACC |
| | | | CAGAAACGCTGGTGAAAGTAAAAGATGCTGAAGATCA |
| | | | GTTGGGTGCACGAGTGGGTTACATCGAACTGGATCTC |
| | | | AACAGCGGTAAGATCCTTGAGAGTTTTCGCCCCGAAG |
| | | | AACGTTTCCCAATGATGAGCACTTTTAAAGTTCTGCT |
| | | | ATGTGGCGCGGTATTATCCCGTGTTGACGCCGGGCAA |
| | | | GAGCAACTCGGTCGCCGCATACACTATTCTCAGAATG |
| | | | ACTTGGTTGAGTACTCACCAGTCACAGAAAAGCATCT |
| | | | TACGGATGGCATGACAGTAAGAGAATTATGCAGTGCT |
| | | | GCCATAACCATGAGTGATAACACTGCGGCCAACTTAC |
| | | | TTCTGACAACGATCGGAGGACCGAAGGAGCTAACCGC |
| | | | TTTTTTGCACAACATGGGGGATCATGTAACTCGCCTT |
| | | | GATCGTTGGGAACCGGAGCTGAATGAAGCCATACCAA |
| | | | ACGACGAGCGTGACACCACGATGCCTGTAGCAATGGC |
| | | | AACAACGTTGCGCAAACTATTAACTGGCGAACTACTT |
| | | | ACTCTAGCTTCCCGGCAACAATTAATAGACTGGATGG |
| | | | AGGCGGATAAAGTTGCAGGACCACTTCTGCGCTCGGC |
| | | | CCTTCCGGCTGGCTGGTTTATTGCTGATAAATCTGGA |
| | | | GCCGGTGAGCGTGGGTCTCGCGGTATCATTGCAGCAC |
| | | | TGGGGCCAGATGGTAAGCCCTCCCGTATCGTAGTTAT |
| | | | CTACACGACGGGGAGTCAGGCAACTATGGATGAACGA |
| | | | AATAGACAGATCGCTGAGATAGGTGCCTCACTGATTA |
| | | | AGCATTGGTAACTGTCAGACCAAGTTTACTCATATAT |
| | | | ACTTTAGATTGATTTCCTTAGGACTGAGCGTCAACCC |
| | | | CGTAGAAAAGATCAAAGGATCTTCTTGAGATCCTTTT |
| | | | TTTCTGCGCGTAATCTGCTGCTTGCAAACAAAAAAAC |
| | | | CACCGCTACCAGCGGTGGTTTGTTTGCCGGATCAAGA |
| | | | GCTACCAACTCTTTTTCCGAAGGTAACTGGCTTCAGC |
| | | | AGAGCGCAGATACCAAATACTGTCCTTCTAGTGTAGC |
| | | | CGTAGTTAGGCCACCACTTCAAGAACTCTGTAGCACC |
| | | | GCCTACATACCTCGCTCTGCTAATCCTGTTACCAGTG |
| | | | GCTGCTGCCAGTGGCGATAAGTCGTGTCTTACCGGGT |
| | | | TGGACTCAAGACGATAGTTACCGGATAAGGCGCAGCG |
| | | | GTCGGGCTGAACGGGGGGTTCGTGCACACAGCCCAGC |
| | | | TTGGAGCGAACGACCTACACCGAACTGAGATACCTAC |
| | | | AGCGTGAGCTATGAGAAAGCGCCACGCTTCCCGAAGG |
| | | | GAGAAAGGCGGACAGGTATCCGGTAAGCGGCAGGGTC |
| | | | GGAACAGGAGAGCGCACGAGGGAGCTTCCAGGGGGAA |
| | | | ACGCCTGGTATCTTTATAGTCCTGTCGGGTTTCGCCA |
| | | | CCTCTGACTTGAGCGTCGATTTTTGTGATGCTCGTCA |
| | | | GGGGGGCGGAGCCTATGGAAAAACGCCAGCAACGCGG |
| | | | CCTTTTTACGGTTCCTGGCCTTTTGCTGGCCTTTTGC |
| | | | TCACATGTTCTTTCCTGCGTTATCCCCTGATTCTGTG |
| | | | GATAACCGTATTACCGCCTTTGAGTGAGCTGATACCG |
| | | | CTCGCCGCAGCCGAACGACCGAGCGCAGCGAGTCAGT |
| | | | GAGCGAGGAAGCGGAAGAGCGCCTGATGCGGTATTTT |
| | | | CTCCTTACGCATCTGTGCGGTATTTCACACCGCATAT |
| | | | AAGGTGCACTGTGACTGGGTCATGGCTGCGCCCCGAC |
| | | | ACCCGCCAACACCCGCTGACGCGCCCTGACGGGCTTG |
| | | | TCTGCTCCCGGCATCCGCTTACAGACAAGCTGTGACC |
| | | | GTCTCCGGGAGCTGCATGTGTCAGAGGTTTTCACCGT |
| | | | CATCACCGAAACGCGCGAGGCAGCTGCGGTAAAGCTC |
| | | | ATCAGCGTGGTCGTGCAGCGATTCACAGATGTCTGCC |
| | | | TGTTCATCCGCGTCCAGCTCGTTGAGTTTCTCCAGAA |
| | | | GCGTTAATGTCTGGCTTCTGATAAAGCGGGCCATGTT |
| | | | AAGGGCGGTTTTTTCCTGTTTGGTCACTGATGCCTCC |
| | | | GTGTAAGGGGGATTTCTGTTCATGGGGGTAATGATAC |
| | | | CGATGAAACGAGAGAGGATGCTCACGATACGGGTTAC |
| | | | TGATGATGAACATGCCCGGTTACTGGAACGTTGTGAG |
| | | | GGTAAACAACTGGCGGTATGGATGCGGCGGGACCAGA |
| | | | GAAAAATCACTCAGGGTCAATGCCAGCGCTTCGTTAA |
| | | | TACAGATGTAGGTGTTCCACAGGGTAGCCAGCAGCAT |
| | | | CCTGCGATGCAGATCCGGAACATAATGGTGCAGGGCG |
| | | | CTGACTTCCGCGTTTCCAGACTTTACGAAACACGGAA |
| | | | ACCGAAGACCATTCATGTTGTTGCTCAGGTCGCAGAC |
| | | | GTTTTGCAGCAGCAGTCGCTTCACGTTCGCTCGCGTA |
| | | | TCGGTGATTCATTCTGCTAACCAGTAAGGCAACCCCG |
| | | | CCAGCCTAGCCGGGTCCTCAACGACAGGAGCACGATC |
| | | | ATGCGCACCCGTGGCCAGGACCCAACGCTGCCCGAAA |
| | | | TT |
| 47. | pTRIM20 | site 1 (IS7) | GCCATATTCAACGGGAAACGTCTTGCTCGAGGCCGCG |
| | | | ATTAAATTCCAACATGGATGCTGATTTATATGGGTAT |
| | | | AAATGGGCTCGCGATAATGTCGGGCAATCAGGTGCGA |
| | | | CAATCTATCGATTGTATGGGAAGCCCGATGCGCCAGA |
| | | | GTTGTTTCTGAAACATGGCAAAGGTAGCGTTGCCAAT |
| | | | GATGTTACAGATGAGATGGTCAGACTAAACTGGCTGA |
| | | | CGGAATTTATGCCTCTTCCGACCATCAAGCATTTTAT |
| | | | CCGTACTCCTGATGATGCATGGTTACTCACCACTGCG |
| | | | ATCCCCGGGAAAACAGCATTCCAGGTATTAGAAGAAT |
| | | | ATCCTGATTCAGGTGAAAATATTGTTGATGCGCTGGC |
| | | | AGTGTTCCTGCGCCGGTTGCATTCGATTCCTGTTTGT |
| | | | AATTGTCCTTTTAACAGCGATCGCGTATTTCGTCTCG |
| | | | CTCAGGCGCAATCACGAATGAATAACGGTTTGGTTGA |
| | | | TGCGAGTGATTTTGATGACGAGCGTAATGGCTGGCCT |
| | | | GTTGAACAAGTCTGGAAAGAAATGCATAAGCTTTTGC |
| | | | CATTCTCACCGGATTCAGTCGTCACTCATGGTGATTT |
| | | | CTCACTTGATAACCTTATTTTTGACGAGGGGAAATTA |
| | | | ATAGGTTGTATTGATGTTGGACGAGTCGGAATCGCAG |
| | | | ACCGATACCAGGATCTTGCCATCCTATGGAACTGCCT |
| | | | CGGTGAGTTTTCTCCTTCATTACAGAAACGGCTTTTT |
| | | | CAAAAATATGGTATTGATAATCCTGATATGAATAAAT |
| | | | TGCAGTTTCATTTGATGCTCGATGAGTTTTTCTAATA |
| | | | AAAAATGCCCTCTTGGGTTATCAAGAGGGTCATTATA |
| | | | TTTCGCAAAAAACCCCGCTTCGGCGGGGTTTTTTCGC |
| | | | TTGACGGCTAGCTCAGTCCTAGGTACAGTGCTAGCAA |
| | | | TTAAAGGAGGCCTAATGACCACTGCTGCACCCCAAGA |
| | | | ATTTACTGCTGCTGTTGTTGAAAAATTCGGTCATGAC |
| | | | GTGACCGTGAAGGATATTGACCTTCCAAAGCCAGGGC |
| | | | CACACCAGGCATTGGTGAAGGTACTCACCTCCGGCAT |
| | | | CTGCCACACCGACCTCCACGCCTTGGAGGGCGATTGG |
| | | | CCAGTAAAGCCGGAACCACCATTCGTACCAGGACACG |
| | | | AAGGTGTAGGTGAAGTTGTTGAGCTCGGACCAGGTGA |
| | | | ACACGATGTGAAGGTCGGCGATATTGTCGGCAATGCG |
| | | | TGGCTCTGGTCAGCGTGTGGCACCTGCGAATACTGCA |
| | | | TCACCGGCAGGGAAACTCAGTGCAACGAAGCTGAGTA |
| | | | TGGTGGCTACACCCAAAATGGATCCTTCGGCCAGTAC |
| | | | ATGCTGGTGGATACCCGTTACGCCGCTCGCATCCCAG |
| | | | ACGGCGTGGACTACCTCGAAGCAGCACCAATTCTGTG |
| | | | TGCAGGCGTGACTGTCTACAAGGCACTCAAAGTCTCT |
| | | | GAAACCCGCCCGGGCCAATTCATGGTGATCTCCGGTG |
| | | | TCGGCGGACTTGGCCACATCGCAGTCCAATACGCAGC |
| | | | GGCGATGGGCATGCGTGTCATTGCGGTAGATATTGCC |
| | | | GATGACAAGCTGGAACTTGCCCGTAAGCACGGTGCGG |
| | | | AATTTACCGTGAATGCGCGTAATGAAGATTCAGGCGA |
| | | | AGCTGTACAGAAGTACACCAACGGTGGCGCACACGGC |
| | | | GTGCTTGTGACTGCAGTTCACGAGGCAGCATTCGGCC |
| | | | AGGCACTGGATATGGCTCGACGTGCAGGAACAATTGT |
| | | | GTTCAACGGTCTGCCACCGGGAGAGTTCCCAGCATCC |
| | | | GTGTTCAACATCGTATTCAAGGGCCTGACCATCCGTG |
| | | | GATCCCTCGTGGGAACCCGCCAAGACTTGGCCGAAGC |
| | | | GCTCGATTTCTTTGCACGCGGACTAATCAAGCCAACC |
| | | | GTGAGTGAGTGCTCCCTCGATGAGGTCAATGGTGTGC |
| | | | TTGACCGCATGCGAAACGGCAAGATTGATGGTCGTGT |
| | | | GGCAATTCGCTACTAAGCATGGACGAGCTGTACAAGT |
| | | | AAGACTGCTAAAGCGTCAAAAGGCCGGATTTTCCGGC |
| | | | CTTTTTTATTACTAAACGCAAAAAGGCCATCCGTCAG |
| | | | GATGGCCTTCTGCTTAATTTGATGCCTGGCAGTTTAT |
| | | | GGCGGGCGTCCTGCCCGCCACCCTCCGGGCCGTTGCT |
| | | | TCGCAACGTTCAAATCCGCTCCCGGCGGATTTGTCCT |
| | | | ACTCAGGAGAGCGCTCCTAAGTCATTTAGATATTGGC |
| | | | TGGGATGTTCAGGGTGATTTCGTTAATATGGCGTTGG |
| | | | GAGTGTAAAAGTTGAGCGGTCCATGCGCAGGCGAACT |
| | | | CCAAACCCTCCAGCGAGCGGTTTGCAAAACGTACGAT |
| | | | TTGGTTTGCCCATACGGGCGGCAGAACATGGTCGCCC |
| | | | GCGGCAGACGCGCGTTGATAGTCCAGCTCGGCTTCGT |
| | | | GGCGCCAAACACGAATTAATTGACCGATGGCTGCCGA |
| | | | CTGAATGCGGCCATAAACATTTCCGTTCTGATCTGCC |
| | | | CCATTAGACAGAAAGATCACACGTGGTCCGCGGGCGC |
| | | | GCGCCCCAGGCGTCAGGCGTTGTGACTGCCAGTAGCG |
| | | | AGCAAGACGGCTAGCAATGACAATCGTCCCGGTGATT |
| | | | TCGTCCGCAAGAAAGTTCAGCACACGTTCGTCATCTA |
| | | | CTTCAATTACACACGGTGCGGGCTCATGGGATGTCGC |
| | | | GGGCAGGATAACCGCCGCATGAATACCCCCAGTGTTT |
| | | | TCTCCAGCTCCCCAATCGAATACAGCGTCGATCGTAG |
| | | | CAGGGTCACGCGGATCAAGAGGTAAGGCGATCGGCGG |
| | | | GGTAAAGTCTGCGCCAGCTAAACGACGCGACTCATTT |
| | | | ACTGCCTGTTCAAACTGTGCTAATGCGGCGGCGCTAC |
| | | | GAAAACCAATAATTACCTCCGATCCGCATGTGCGCAG |
| | | | CATACCCGTTAAAGCCATTACCTCCTCAATCTGATCC |
| | | | CCGGCACAGATAAGGGTGGTACGGCCAGAGGCATCGA |
| | | | TAGTGCGCAGATCAGTGCGCGCTAACAGACTCGTTTC |
| | | | CGAACAGGCCGGCAGCTCCATTCCATGGGTCACCTCA |
| | | | ATCGTTTCCCCAGATAATGCTGCAGACTCAGCAGAAG |
| | | | CCAAGAAGACCGCGGCATCGGCGACATCGCCGACGGA |
| | | | AGGGAAGCGGCGTTCTAATGCCCCTTGGTCGTTTGCG |
| | | | CGACACAGGCGCATCGTGTTCAAGAAATGGTGCGCCG |
| | | | TGTCACCCTCAGGACGGCCCTTCAGCTGATCCATACG |
| | | | TTGAAATACAGTGCGAATGCGGTCCGATTCGATGGGA |
| | | | CCCGGAAAGATTGTATTTACGCGAATTCCACGCGCGC |
| | | | CCAGTTCGCGCGCAGCCAGCTGGCTTAACGCATTCAG |
| | | | TGCGGCTTTGGGGGTCACATATGGGATACGTCCGTAG |
| | | | TATTCGGCACGAGAAAAGATGGTGGACACATTGATAA |
| | | | CAGCTGATCCAACGGGCATGTGCGGGGCCGCAATGCG |
| | | | CATCAGATGCCAGCCCATTCCAAGAAGATTGGCGATA |
| | | | CTAGCGTGCAATGTTTCCTCTGCTCCGGGACCCAATT |
| | | | CTGCTTCAGTTAAAGGGATTTCCGCTAAACGACGCTG |
| | | | AGCACCAGCGCTACCGGCGTTGTTGACAAGAATGTCA |
| | | | ATCTGGCCGTGGCGAGCAACAATAGCCTCAATGCCTG |
| | | | CGCGCACGGCAACTGGGTCGCTTCCGTCCATAACTTC |
| | | | CAAGTCAATGCGTTTAGCGGGGACGCCGGCCTCAGCT |
| | | | TGCATACGCTCGGCCAGCGCAGTCAGCTTAGCACGAT |
| | | | TACGTCCAGAGATAATGACTGTCGCACCCTCTGCCAG |
| | | | GAAGCGACGGGTTAATTCGGAGCCAATATTGCCGGCA |
| | | | CCTCCCGTAATCAAGGCAATTTTTCCCGCCAAGCGCC |
| | | | CTGTTCCTGACATTGAGTACCTCCTTTCTTAATTATA |
| | | | CCGTAATCGCGCGACCGCGGTGGATACGTCCGGCATA |
| | | | ACGAGTGTCCGCTTCGGGTGGCAACGGTGCGGTAACT |
| | | | AACAGCACGGCTTCGATGAAGCGCTCCAATTCCTGTT |
| | | | GACGTTCGTGAGGGTCACGCGGCTCCTCTGCGCGAGC |
| | | | ACGACGAGTTAAATCGACTTGATTGATCAGGATGCGC |
| | | | TGCGCTGTGCGTTCAGACTCTACACCGATCGTTGCGG |
| | | | TGAAAGCATGAAGCGTTGTCTTAATGAAGTTGGCCAA |
| | | | GGCGAACTGCTCGGTTGTGCTAGTTGCCGTAGTCTCG |
| | | | GGCGTGACTAAGGCAAGACGTGCACCGTCAGACAGGG |
| | | | CGATCCAACGTGCTACACGGAAGTGATGCGTTAACTG |
| | | | ATGCTCACATAACTCAGCGAATTCTGCCTCGCTCAAG |
| | | | ACGGTAGACCAATCGCTGTCCTTGCGGCCTACAAGAG |
| | | | GAACAGTGGGCAATGGACGAAAAGGGGTACAGACTAC |
| | | | GGGACCTGGACGTCCGTAACGAGTAATCGCTTGGTCA |
| | | | ATCGCGGCCTCGATCTGGTCACCAGCCACAATCGTCA |
| | | | TCAAGCGTCCTGCCTCTACGTGGTCGTGCAGTAAACG |
| | | | ACGCATGGTTTCAGCTCCAGTTTCCGTTTCTACGATC |
| | | | ATAACTACCTGGCGTGCCCCGTAGCGCTCTAAGTAAG |
| | | | CGCGTGCAAGCAAGTTTAAATGCTCAGTTAAATGTTC |
| | | | ACCGATCAGATAGACTGTGGAGCCGACAAGCTCTGCT |
| | | | AAACGTTCCGGCGAAGGCAGGCCAAACAATTCTCCCC |
| | | | CTGTCGGGGTGCGTTCGTAGCGCAAACCTCCAGACGG |
| | | | ATGGAAAGTCTCGCCTGAGACAACACGGTCCGCCAAA |
| | | | TAATAAACCGTCGCCATCGCAACATCAAATTCCGTAG |
| | | | GCATGCGCTGAAGATAAAGCATTCCCATAATTCCATC |
| | | | GCGTACCTTACGAGCCTCGCGGTCGATCTGGGCGCGG |
| | | | GTGAAAAAAGGGTCTGGTGGGTTCGGCAAATTGGCAA |
| | | | AAATGTCTGCTGGCAGAACATATCCACCATTGTGCAA |
| | | | ACGAGCAAGTAATTTCGCCGCAATGGAGCGATTTAAT |
| | | | AAAGCACTGGACGAAGACGCTGCAGGGTCTCCCTCAC |
| | | | TGCGGAAGCGACGCGCCAGCTCACGAAGTGCGGTAGG |
| | | | GGCAGCGGGGTTTTGTTCAAGCGCCGCGACGTCATTC |
| | | | GGCAACAACAATTCAACCAACTCATGCATCGAACGTT |
| | | | CATCGGTACGAGCTGCCGCGATTAACGCAGCATGAAG |
| | | | CTCATTTAAACGCTTGTTCTCCAAGATAAGGCGTGCA |
| | | | CGGCGAGCGAACAATCCAGGACGCTCACCAGTTCCGC |
| | | | GCAGACGATCTCCCTCTACGGGTCCAGGTGCAATAGC |
| | | | ATTGATCTGAATCTCGGGTCCCAAAAAGCGCGCAAAG |
| | | | ACTTCAGCCATAGCGCGCTGGCCGGCTTTACTAACAG |
| | | | CATAGTCGGCACGGTTGGGATACGGAATTGCTGCGTC |
| | | | TTTCTCCCCTCCGAAATACGAGCTTACGTTGAGGATG |
| | | | TACCCCGACCCCTGCTTCTTCATCAGTGGTGCCAGCT |
| | | | TACGCATAAGGCTGTAGTTCGAGATGAGGTTTGCGAA |
| | | | CAGCGTATGACGCCAACCCTCCACGGGCATGTCGATA |
| | | | ACCATCTCCTCTACACCCGCAATGCCGGCGTTATTGA |
| | | | TTAAATAGTCAACGGTACCGAAAGCTGACAATGTGCG |
| | | | TTCAACCAAGTCTGCAAGTTGGGCCTCACTTGAAACG |
| | | | TCGCACCCTGGTGCGATATGAACGCGATCCTCCACAT |
| | | | CTGTGTAGCCAACTTCCGCTAATTCACTCTGAATCAT |
| | | | AGCTTGCATCTGTTCCAATTTGTGGCGATCACGTGCC |
| | | | GCCAGCATCACACGTGCACCAGATAAAGCCAATAAGC |
| | | | GACCAATCTGCCCACCAATGCCGGCAGATCCACCCGT |
| | | | AATCAGGGCGACCTTGCCCAAATGCAACCCAATCAGT |
| | | | GATTCTGCCCATCCAACAGAGGCGCTACGTGCGCCCG |
| | | | TCGTCGCCGACATTAATACCTCCTGTCCTTGTTGGTC |
| | | | AATTGCTCGTGAAAACACCTAAACGGACTGGCTGTTT |
| | | | CCTGTGTGAGCTAGCATTATACCTAGGACTGAGCTAG |
| | | | CTGTCAATTAATAAGATGATCTTCTTGAGATCGTTTT |
| | | | GGTCTGCGCGTAATCTCTTGCTCTGAAAACGAAAAAA |
| | | | CCGCCTTGCAGGGCGGTTTTTCGAAGGTTCTCTGAGC |
| | | | TACCAACTCTTTGAACCGAGGTAACTGGCTTGGAGGA |
| | | | GCGCAGTCACCAAAACTTGTCCTTTCAGTTTAGCCTT |
| | | | AACCGGCGCATGACTTCAAGACTAACTCCTCTAAATC |
| | | | AATTACCAGTGGCTGCTGCCAGTGGTGCTTTTGCATG |
| | | | TCTTTCCGGGTTGGACTCAAGACGATAGTTACCGGAT |
| | | | AAGGCGCAGCGGTCGGACTGAACGGGGGGTTCGTGCA |
| | | | TACAGTCCAGCTTGGAGCGAACTGCCTACCCGGAACT |
| | | | GAGTGTCAGGCGTGGAATGAGACAAACGCGGCCATAA |
| | | | CAGCGGAATGACACCGGTAAACCGAAAGGCAGGAACA |
| | | | GGAGAGCGCACGAGGGAGCCGCCAGGGGGAAACGCCT |
| | | | GGTATCTTTATAGTCCTGTCGGGTTTCGCCACCACTG |
| | | | ATTTGAGCGTCAGATTTCGTGATGCTTGTCAGGGGGG |
| | | | CGGAGCCTATGGAAAAACGGCTTTGCCGCGGCCCTCT |
| | | | CACTTCCCTGTTAAGTATCTTCCTGGCATCTTCCAGG |
| | | | AAATCTCCGCCCCGTTCGTAAGCCATTTCCGCTCGCC |
| | | | GCAGTCGAACGACCGAGCGTAGCGAGTCAGTGAGCGA |
| | | | GGAAGCGGAATATATCCTGTATCACATATTCTGCTGA |
| | | | CGCACCGGTGCAGCCTTTTTTCTCCTGCCACATGAAG |
| | | | CACTTCACTGACACCCTCATCAGTGCCAACATAGTAA |
| | | | GCCAGTATACACTCCGCTAGCGCTGAGGTCTGCCTCG |
| | | | TGAAGAAGGTGTTGCTGACTCATACCAGGCCTGAATC |
| | | | GCCCCATCATCCAGCCAGAAAGTGAGGGAGCCACGGT |
| | | | TGATGAGAGCTTTGTTGTAGGTGGACCAGTTGGTGAT |
| | | | TTTGAACTTTTGCTTTGCCACGGAACGGTCTGCGTTG |
| | | | TCGGGAAGATGCGTGATCTGATCCTTCAACTCAGCAA |
| | | | AAGTTCGATTTATTCAACAAAGCCACGTTGTGTCTCA |
| | | | AAATCTCTGATGTTACATTGCACAAGATAAAAATATA |
| | | | TCATCATGAACAATAAAACTGTCTGCTTACATAAACA |
| | | | GTAATACAAGGGGTGTTATGA |
| 48. | IMMO | | GAACCCCCAGGATGCGCATAGCGCTTATATTCGCGGT |
| | | | GACGTGGAGTTGGTGCGGATTCGTGATGCCGAAGGGC |
| | | | GAATTGCGGCAGAAGGGGCGTTGCCTTATCCACCTGG |
| | | | CGTGCTTTGCGTGGTACCCGGGGAAGTCTGGGGTGGG |
| | | | GCGGTTCAACGTTATTTCCTTGCACTGGAAGAAGGGG |
| | | | TGAATTTGTTGCCGGGATTTTCGCCGGAGCTGCAAGG |
| | | | TGTTTATAGCGAAACCGATGCGGATGGCGTGAAACGG |
| | | | TTGTACGGTTATGTGTTGAAGTAAGAATAAAAAAAAC |
| | | | GGGTCACCTTCTGGCGACCCGTTTTTCTTTGCGTAAT |
| | | | TAGTGGCTAACCGTCTGTGTGCCTGTCGGGACACGAA |
| | | | CGTGTTTATATTTGAACATCGCCATGAACGCGAAGGC |
| | | | CAGAACCACGGAGTAACCAGCGAAAATCAACCATACG |
| | | | GTCTGCCAGTCGGTAATGCCGTTTTGGGTGTACATCT |
| | | | CAACAACTTTACCGCTCACGATGCCGCCGAGGATACA |
| | | | GCCGAAGCCGTTAGTCATCATCAGGAACATCCCTTGT |
| | | | GCACTGGCGCGAATTGCCGGGCTAACTTCTTTTTCGA |
| | | | CAAACACCGAACCAGAGATGTTGAAGAAGTCGAATGC |
| | | | GCAACCGTAAACGATCATCGACAGTACCAGCAGTACA |
| | | | GTACCTCACACTGCTTCCGGTAGTCAATAAACCGGTA |
| | | | AACCAGCAATAGACATAAGCGGTGCATAATGTGCCTG |
| | | | TCAAATGGACGAAGCAGGGATTCTGCAAACCCTATGC |
| | | | TACTCCGTCAAGCCGTCAATTGTCTGATTCGTTACCA |
| | | | ATTATGACAACTTGACGGCTACATCATTCACTTTTTC |
| | | | TTCACAACCGGCACGGAACTCGCTCGGGCTGGCCCCG |
| | | | GTGCATTTTTTAAATACCCGCGAGAAATAGAGTTGAT |
| | | | CGTCAAAACCAACATTGCGACCGACGGTGGCGATAGG |
| | | | CATCCGGGTGGTGCTCAAAAGCAGCTTCGCCTGGCTG |
| | | | ATACGTTGGTCCTCGCGCCAGCTTAAGACGCTAATCC |
| | | | CTAACTGCTGGCGGAAAAGATGTGACAGACGCGACGG |
| | | | CGACAAGCAAACATGCTGTGCGACGCTGGCGATATCA |
| | | | AAATTGCTGTCTGCCAGGTGATCGCTGATGTACTGAC |
| | | | AAGCCTCGCGTACCCGATTATCCATCGGTGGATGGAG |
| | | | CGACTCGTTAATCGCTTCCATGCGCCGCAGTAACAAT |
| | | | TGCTCAAGCAGATTTATCGCCAGCAGCTCCGAATAGC |
| | | | GCCCTTCCCCTTGCCCGGCGTTAATGATTTGCCCAAA |
| | | | CAGGTCGCTGAAATGCGGCTGGTGCGCTTCATCCGGG |
| | | | CGAAAGAACCCCGTATTGGCAAATATTGACGGCCAGT |
| | | | TAAGCCATTCATGCCAGTAGGCGCGCGGACGAAAGTA |
| | | | AACCCACTGGTGATACCATTCGCGAGCCTCCGGATGA |
| | | | CGACCGTAGTGATGAATCTCTCCTGGCGGGAACAGCA |
| | | | AAATATCACCCGGTCGGCAAACAAATTCTCGTCCCTG |
| | | | ATTTTTCACCACCCCCTGACCGCGAATGGTGAGATTG |
| | | | AGAATATAACCTTTCATTCCCAGCGGTCGGTCGATAA |
| | | | AAAAATCGAGATAACCGTTGGCCTCAATCGGCGTTAA |
| | | | ACCCGCCACCAGATGGGCATTAAACGAGTATCCCGGC |
| | | | AGCAGGGGATCATTTTGCGCTTCAGCCATACTTTTCA |
| | | | TACTCCCGCCATTCAGAGAAGAAACCAATTGTCCATA |
| | | | TTGCATCAGACATTGCCGTCACTGCGTCTTTTACTGG |
| | | | CTCTTCTCGCTAACCAAACCGGTAACCCCGCTTATTA |
| | | | AAAGCATTCTGTAACAAAGCGGGACCAAAGCCATGAC |
| | | | AAAAACGCGTAACAAAAGTGTCTATAATCACGGCAGA |
| | | | AAAGTCCACATTGATTATTTGCACGGCGTCACACTTT |
| | | | GCTATGCCATAGCATTTTTATCCATAAGATTAGCGGA |
| | | | TCCTACCTGACGCTTTTTATCGCAACTCTGGACAATG |
| | | | TCTCCATACCCGTTTTTTTGGGCGACCTCGTCGGAGG |
| | | | TTGTATGTCCGGTGTTCCGTGACGTCATCGGGCATTC |
| | | | ATCATTCATAGAATGTGTTACGGAGGAAACAAGTAAT |
| | | | GGCACTTAGCACCGCAACCAAGGCCGCGACGGACGCG |
| | | | CTGGCTGCCAATCGGGCACCCACCAGCGGGAATGCAC |
| | | | AGGAAGTGCACCGTTGGCTCCAGAGCTTCAACTGGGA |
| | | | TTTCAAGAACAACCGGACCAAGTACGCCACCAAGTAC |
| | | | AAGATGGCGAACGAGACCAAGGAACAGTTCAAGCTGA |
| | | | TCGCCAAGGAATATGCGCGCATGGAGGCAGTCAAGGA |
| | | | CGAAAGGCAGTTCGGTAGCCTGCAGGATGCGCTGACC |
| | | | CGCCTCAACGCCGGTGTTCGCGTTCATCCGAAGTGGA |
| | | | ACGAGACCATGAAAGTGGTTTCGAACTTCCTGGAAGT |
| | | | GGGCGAATACAACGCCATCGCCGCTACCGGGATGCTG |
| | | | TGGGATTCCGCCCAGGCGGCGGAACAGAAGAACGGCT |
| | | | ATCTGGCCCAGGTGTTGGATGAAATCCGCCACACCCA |
| | | | CCAGTGTGCCTACGTCAACTACTACTTCGCGAAGAAC |
| | | | GGCCAGGACCCGGCCGGTCACAACGATGCTCGCCGCA |
| | | | CCCGTACCATCGGTCCGCTGTGGAAGGGCATGAAGCG |
| | | | CGTGTTTTCCGACGGCTTCATTTCCGGCGACGCCGTG |
| | | | GAATGCTCCCTCAACCTGCAGCTGGTGGGTGAGGCCT |
| | | | GCTTCACCAATCCGCTGATCGTCGCAGTGACCGAATG |
| | | | GGCTGCCGCCAACGGCGATGAAATCACCCCGACGGTG |
| | | | TTCCTGTCGATCGAGACCGACGAACTGCGCCACATGG |
| | | | CCAACGGTTACCAGACCGTCGTTTCCATCGCCAACGA |
| | | | TCCGGCTTCCGCCAAGTATCTCAACACGGACCTGAAC |
| | | | AACGCCTTCTGGACCCAGCAGAAGTACTTCACGCCGG |
| | | | TGTTGGGCATGCTGTTCGAGTATGGCTCCAAGTTCAA |
| | | | GGTCGAGCCGTGGGTCAAGACGTGGAACCGCTGGGTG |
| | | | TACGAGGACTGGGGCGGCATCTGGATCGGCCGTCTGG |
| | | | GCAAGTACGGGGTGGAGTCGCCGCGCAGCCTCAAGGA |
| | | | CGCCAAGCAGGACGCTTACTGGGCTCACCACGACCTG |
| | | | TATCTGCTGGCTTATGCGCTGTGGCCGGGCGGCTTCT |
| | | | TCCGTCTGGCGCTGCCGGATCAGGAAGAAATGGAGTG |
| | | | GTTCGAGGCCAACTACCCCGGCTGGTACGACCACTAC |
| | | | GGCAAGATCTACGAGGAATGGCGCGCCCGCGGTTGCG |
| | | | AGGATCCGTCCTCGGGCTTCATCCCGCTGATGTGGTT |
| | | | CATCGAAAACAACCATCCCATCTACATCGATCGCGTG |
| | | | TCGCAAGTGCCGTTCTGCCCGAGCTTGGCCAAGGGCG |
| | | | CCAGCACCCTGCGCGTGCACGAGTACAACGGCCAGAT |
| | | | GCACACCTTCAGCGACCAGTGGGGCGAGCGCATGTGG |
| | | | CTGGCCGAGCCGGAGCGCTACGAGTGCCAGAACATCT |
| | | | TCGAACAGTACGAAGGACGCGAACTGTCGGAAGTGAT |
| | | | CGCCGAACTGCACGGGCTGCGCAGTGATGGCAAGACC |
| | | | CTGATCGCCCAGCCGCATGTCCGTGGCGACAAGCTGT |
| | | | GGACGTTGGACGATATCAAACGCCTGAACTGCGTCTT |
| | | | CAAGAACCCGGTGAAGGCATTCAATTGAAACGGGTGT |
| | | | CGGGCTCCGTCACAGGGGGGGGCCCGACGCACGATCG |
| | | | TTCGATCAACCTCAAACCAAAAAGGAACATCGATATG |
| | | | AGCATGTTAGGAGAAAGACGCCGCGGTCTGACCGATC |
| | | | CGGAAATGGCGGCCGTCATTTTGAAGGCGCTTCCTGA |
| | | | AGCTCCGCTGGACGGCAACAACAAGATGGGTTATTTC |
| | | | GTCACCCCCCGCTGGAAACGCTTGACGGAATATGAAG |
| | | | CCCTGACCGTTTATGCGCAGCCCAACGCCGACTGGAT |
| | | | CGCCGGCGGCCTGGACTGGGGCGACTGGACCCAGAAA |
| | | | TTCCACGGCGGCCGCCCTTCCTGGGGCAACGAGACCA |
| | | | CGGAGCTGCGCACCGTCGACTGGTTCAAGCACCGTGA |
| | | | CCCGCTCCGCCGTTGGCATGCGCCGTACGTCAAGGAC |
| | | | AAGGCCGAGGAATGGCGCTACACCGACCGCTTCCTGC |
| | | | AGGGTTACTCCGCCGACGGTCAGATCCGGGCGATGAA |
| | | | CCCGACCTGGCGGGACGAGTTCATCAACCGGTATTGG |
| | | | GGCGCCTTCCTGTTCAACGAATACGGATTGTTCAACG |
| | | | CTCATTCGCAGGGCGCCCGGGAGGCGCTGTCGGACGT |
| | | | AACCCGCGTCAGCCTGGCTTTCTGGGGCTTCGACAAG |
| | | | ATCGACATCGCCCAGATGATCCAACTCGAACGGGGTT |
| | | | TCCTCGCCAAGATCGTACCCGGTTTCGACGAGTCCAC |
| | | | AGCGGTGCCGAAGGCCGAATGGACGAACGGGGAGGTC |
| | | | TACAAGAGCGCCCGTCTGGCCGTGGAAGGGCTGTGGC |
| | | | AGGAGGTGTTCGACTGGAACGAGAGCGCTTTCTCGGT |
| | | | GCACGCCGTCTATGACGCGCTGTTCGGTCAGTTCGTC |
| | | | CGCCGCGAGTTCTTTCAGCGGCTGGCTCCCCGCTTCG |
| | | | GCGACAATCTGACGCCATTCTTCATCAACCAGGCCCA |
| | | | GACATACTTCCAGATCGCCAAGCAGGGCGTACAGGAT |
| | | | CTGTATTACAACTGTCTGGGTGACGATCCGGAGTTCA |
| | | | GCGATTACAACCGTACCGTGATGCGCAACTGGACCGG |
| | | | CAAGTGGCTGGAGCCCACGATCGCCGCTCTGCGCGAC |
| | | | TTCATGGGGCTGTTTGCGAAGCTGCCGGCGGGCACCA |
| | | | CTGACAAGGAAGAAATCACCGCGTCCCTGTACCGGGT |
| | | | GGTCGACGACTGGATCGAGGACTACGCCAGCAGGATC |
| | | | GACTTCAAGGCGGACCGCGATCAGATCGTTAAAGCGG |
| | | | TTCTGGCAGGATTGAAATAATAGAGGAACTATTACGA |
| | | | TGAGCGTAAACAGCAACGCATACGACGCCGGCATCAT |
| | | | GGGCCTGAAAGGCAAGGACTTCGCCGATCAGTTCTTT |
| | | | GCCGACGAAAACCAAGTGGTCCATGAAAGCGACACGG |
| | | | TCGTTCTGGTCCTCAAGAAGTCGGACGAGATCAATAC |
| | | | CTTTATCGAGGAGATCCTTCTGACGGACTACAAGAAG |
| | | | AACGTCAATCCGACGGTAAACGTGGAAGACCGCGCGG |
| | | | GTTACTGGTGGATCAAGGCCAACGGCAAGATCGAGGT |
| | | | CGATTGCGACGAGATTTCCGAGCTGTTGGGGCGGCAG |
| | | | TTCAACGTCTACGACTTCCTCGTCGACGTTTCCTCCA |
| | | | CCATCGGCCGGGCCTATACCCTGGGCAACAAGTTCAC |
| | | | CATTACCAGTGAGCTGATGGGCCTGGACCGCAAGCTC |
| | | | GAAGACTCTCACGCTTAAGGAGAATGACATGGCGAAA |
| | | | CTGGGTATACACAGCAACGACACCCGCGACGCCTGGG |
| | | | TGAACAAGATCGCGCAGCTCAACACCCTGGAAAAAGC |
| | | | GGCCGAGATGCTGAAGCAGTTCCGGATGGACCACACC |
| | | | ACGCCGTTCCGCAACAGCTACGAACTGGACAACGACT |
| | | | ACCTCTGGATCGAGGCCAAGCTCGAAGAGAAGGTCGC |
| | | | CGTCCTCAAGGCAGAAGCCTTCAACGAGGTGGACTTC |
| | | | CGTCATAAGACCGCTTTCGGCGAGGATGCCAAGTCCG |
| | | | TTCTGGACGGCACCGTCGCGAAGATGAACGCGGCCAA |
| | | | GGACAAGTGGGAGGCGGAGAAGATCCATATCGGTTTC |
| | | | CGCCAGGCCTACAAGCCGCCGATCATGCCGGTGAACT |
| | | | ATTTCCTGGACGGCGAGCGTCAGTTGGGGACCCGGCT |
| | | | GATGGAACTGCGCAACCTCAACTACTACGACACGCCG |
| | | | CTGGAAGAACTGCGCAAACAGCGCGGTGTGCGGGTGG |
| | | | TGCATCTGCAGTCGCCGCACTGAAGGGAGGAAGTCTC |
| | | | GCCCTGGACGCGACGGCATCGCCGTGAAGTCCAGGGG |
| | | | GCAGGGATGCCGTTCCGGGCCGGCAGGCTGGCCCGGA |
| | | | ATCTCTGGTTTTCAGGGGGCGTGCCGGTCCACGGCTC |
| | | | CCCCCTCCATCTTTCGTAAGGAAATCACCATGGTCGA |
| | | | ATCGGCATTTCAGCCATTTTCGGGCGACGCAGACGAA |
| | | | TGGTTCGAGGAACCACGGCCCCAGGCCGGTTTCTTCC |
| | | | CTTCCGCGGACTGGCATCTGCTCAAACGGGACGAGAC |
| | | | CTACGCAGCCTATGCCAAGGATCTCGATTTCATGTGG |
| | | | CGGTGGGTCATCGTCCGGGAAGAAAGGATCGTCCAGG |
| | | | AGGGTTGCTCGATCAGCCTGGAGTCGTCGATCCGCGC |
| | | | CGTGACGCACGTACTGAATTATTTTGGTATGACCGAA |
| | | | CAACGCGCCCCGGCAGAGGACCGGACCGGCGGAGTTC |
| | | | AACATTGAACAGGTAAGTTTATGCAGCGAGTTCACAC |
| | | | TATCACGGCGGTGACGGAGGATGGCGAATCGCTCCGC |
| | | | TTCGAATGCCGTTCGGACGAGGACGTCATCACCGCCG |
| | | | CCCTGCGCCAGAACATCTTTCTGATGTCGTCCTGCCG |
| | | | GGAGGGCGGCTGTGCGACCTGCAAGGCCTTGTGCAGC |
| | | | GAAGGGGACTACGACCTCAAGGGCTGCAGCGTTCAGG |
| | | | CGCTGCCGCCGGAAGAGGAGGAGGAAGGGTTGGTGTT |
| | | | GTTGTGCCGGACCTACCCGAAGACCGACCTGGAAATC |
| | | | GAACTGCCCTATACCCATTGCCGCATCAGTTTTGGTG |
| | | | AGGTCGGCAGTTTCGAGGCGGAGGTCGTCGGCCTCAA |
| | | | CTGGGTTTCGAGCAACACCGTCCAGTTTCTTTTGCAG |
| | | | AAGCGGCCCGACGAGTGCGGCAACCGTGGCGTGAAAT |
| | | | TCGAACCCGGTCAGTTCATGGACCTGACCATCCCCGG |
| | | | CACCGATGTCTCCCGCTCCTACTCGCCGGCGAACCTT |
| | | | CCTAATCCCGAAGGCCGCCTGGAGTTCCTGATCCGCG |
| | | | TGTTACCGGAGGGACGGTTTTCGGACTACCTGCGCAA |
| | | | TGACGCGCGTGTCGGACAGGTCCTCTCGGTCAAAGGG |
| | | | CCACTGGGCGTGTTCGGTCTCAAGGAGCGGGGCATGG |
| | | | CGCCGCGCTATTTCGTGGCCGGCGGCACCGGGTTGGC |
| | | | GCCGGTGGTCTCGATGGTGCGGCAGATGCAGGAGTGG |
| | | | ACCGCGCCGAACGAGACCCGCATCTATTTCGGTGTGA |
| | | | ACACCGAGCCGGAATTGTTCTACATCGACGAGCTCAA |
| | | | ATCCCTGGAACGATCGATGCGCAATCTCACCGTGAAG |
| | | | GCCTGTGTCTGGCACCCGAGCGGGGACTGGGAAGGCG |
| | | | AGCAGGGCTCGCCCATCGATGCGTTGCGGGAAGACCT |
| | | | GGAGTCCTCCGACGCCAACCCGGACATTTATTTGTGC |
| | | | GGTCCGCCGGGCATGATCGATGCCGCCTGCGAGCTGG |
| | | | TACGCAGCCGCGGTATCCCCGGCGAACAGGTCTTCTT |
| | | | CGAAAAATTCCTGCCGTCCGGGGCGGCCTGAACCGGG |
| | | | GAAGTACCGTGACCACCGAGCAGTTCCCGCCCCAATT |
| | | | CCTGCGTGAAATGATCGAGCAGCTGGACGCCAGCATC |
| | | | CAGGAGCTCGCACGCAAGGAAAAGGGACTTGCGGCAT |
| | | | CCCTGGGCACGGGCCGGGTCGCCGAGCTCAAGGAATA |
| | | | CTGGGACCACGTAGTCGGGTCGCCGTAAGCAAACAGC |
| | | | GCAAAACGCAGGATCCACGCCACAATACTGATCATCA |
| | | | TTACGTTCTTAATACCGTAGCGGCTTAAGAAGAACGG |
| | | | GATGGTCAGAATGAACAGGGTTTCAGAGATCTGCGAA |
| | | | ATCGACATGATGATTGACGCATGCTGCACAATAAAGC |
| | | | TGCTGGCAAACATCGGATCTTTGTCGAAGCTGTGCAG |
| | | | GAAGGTATTACCGAACATGTTGGTAATCTGCAGTTCC |
| | | | GCGCCCAGCAGCATTGAGAAGATAAAGAAGATTGCCA |
| | | | TACGCTTGTTTTTAAACAGCGCGAATGCATCGAGGCC |
| | | | CAGCAGGGTTGTCCAGCTCTGATTCGCTTGCTGTTTA |
| | | | GCAACCGGAATATGCGGCAGAGTCAGGGTAAACAGAA |
| | | | CCAGAATGGCGGAAAGTGCTGCGCCAATATACAGCTG |
| | | | CATGTGGCTTAATTCGAAGCCAGACAGGCTCACCACC |
| | | | CACATTGCCATGATAAAGCCGATGGTGCCCCAGATAC |
| | | | GGATTGGC |
| 49. | IMMO | site 2 (SS9) | TTTTCTCAAGTTCTGCCAGCTTCTGCTGGGTTTCTTT |
| | | | GTCTTGTGCAGGATGCTGCATCACCGGCATCATGTTG |
| | | | TCGGCAATAGTGGTCGCCGGTTTAACCAGATACTGGT |
| | | | TTGGGTGATCGTATCCGGCAAAGCCTTTGCGTGCGGT |
| | | | GGCGGTTGACGGGGTATCTGCTGCGTTGGCCATGAGT |
| | | | GGAAGAGCGGCGGCGACAGCTACAACAAGAAGTTTGG |
| | | | GTGAAAACGAAAATTCCATGCAAAATGCTCCGGTTTC |
| | | | ATGTCGTCAAAATGTTGACGTAATTAAGCATTGATAA |
| | | | TTGATAATTGAGATCCCTCTCCCTGACAGGATGATTG |
| | | | CATAAATAATAGTGATGAAAATAAATTATTTATTTAT |
| | | | CCAGAAAATGAATTGGAAAATCAGGAGAGCGTTTTCA |
| | | | ATCCTACCCCTAACTGACTATGGCCTTCTGTAGTCAC |
| | | | ACTGCTTCCGGTAGTCAATAAACCGGTAAACCAGCAA |
| | | | TAGACATAAGCGGTGCATAATGTGCCTGTCAAATGGA |
| | | | CGAAGCAGGGATTCTGCAAACCCTATGCTACTCCGTC |
| | | | AAGCCGTCAATTGTCTGATTCGTTACCAATTATGACA |
| | | | ACTTGACGGCTACATCATTCACTTTTTCTTCACAACC |
| | | | GGCACGGAACTCGCTCGGGCTGGCCCCGGTGCATTTT |
| | | | TTAAATACCCGCGAGAAATAGAGTTGATCGTCAAAAC |
| | | | CAACATTGCGACCGACGGTGGCGATAGGCATCCGGGT |
| | | | GGTGCTCAAAAGCAGCTTCGCCTGGCTGATACGTTGG |
| | | | TCCTCGCGCCAGCTTAAGACGCTAATCCCTAACTGCT |
| | | | GGCGGAAAAGATGTGACAGACGCGACGGCGACAAGCA |
| | | | AACATGCTGTGCGACGCTGGCGATATCAAAATTGCTG |
| | | | TCTGCCAGGTGATCGCTGATGTACTGACAAGCCTCGC |
| | | | GTACCCGATTATCCATCGGTGGATGGAGCGACTCGTT |
| | | | AATCGCTTCCATGCGCCGCAGTAACAATTGCTCAAGC |
| | | | AGATTTATCGCCAGCAGCTCCGAATAGCGCCCTTCCC |
| | | | CTTGCCCGGCGTTAATGATTTGCCCAAACAGGTCGCT |
| | | | GAAATGCGGCTGGTGCGCTTCATCCGGGCGAAAGAAC |
| | | | CCCGTATTGGCAAATATTGACGGCCAGTTAAGCCATT |
| | | | CATGCCAGTAGGCGCGCGGACGAAAGTAAACCCACTG |
| | | | GTGATACCATTCGCGAGCCTCCGGATGACGACCGTAG |
| | | | TGATGAATCTCTCCTGGCGGGAACAGCAAAATATCAC |
| | | | CCGGTCGGCAAACAAATTCTCGTCCCTGATTTTTCAC |
| | | | CACCCCCTGACCGCGAATGGTGAGATTGAGAATATAA |
| | | | CCTTTCATTCCCAGCGGTCGGTCGATAAAAAAATCGA |
| | | | GATAACCGTTGGCCTCAATCGGCGTTAAACCCGCCAC |
| | | | CAGATGGGCATTAAACGAGTATCCCGGCAGCAGGGGA |
| | | | TCATTTTGCGCTTCAGCCATACTTTTCATACTCCCGC |
| | | | CATTCAGAGAAGAAACCAATTGTCCATATTGCATCAG |
| | | | ACATTGCCGTCACTGCGTCTTTTACTGGCTCTTCTCG |
| | | | CTAACCAAACCGGTAACCCCGCTTATTAAAAGCATTC |
| | | | TGTAACAAAGCGGGACCAAAGCCATGACAAAAACGCG |
| | | | TAACAAAAGTGTCTATAATCACGGCAGAAAAGTCCAC |
| | | | ATTGATTATTTGCACGGCGTCACACTTTGCTATGCCA |
| | | | TAGCATTTTTATCCATAAGATTAGCGGATCCTACCTG |
| | | | ACGCTTTTTATCGCAACTCTGGACAATGTCTCCATAC |
| | | | CCGTTTTTTTGGGCGACCTCGTCGGAGGTTGTATGTC |
| | | | CGGTGTTCCGTGACGTCATCGGGCATTCATCATTCAT |
| | | | AGAATGTGTTACGGAGGAAACAAGTAATGGCACTTAG |
| | | | CACCGCAACCAAGGCCGCGACGGACGCGCTGGCTGCC |
| | | | AATCGGGCACCCACCAGCGGGAATGCACAGGAAGTGC |
| | | | ACCGTTGGCTCCAGAGCTTCAACTGGGATTTCAAGAA |
| | | | CAACCGGACCAAGTACGCCACCAAGTACAAGATGGCG |
| | | | AACGAGACCAAGGAACAGTTCAAGCTGATCGCCAAGG |
| | | | AATATGCGCGCATGGAGGCAGTCAAGGACGAAAGGCA |
| | | | GTTCGGTAGCCTGCAGGATGCGCTGACCCGCCTCAAC |
| | | | GCCGGTGTTCGCGTTCATCCGAAGTGGAACGAGACCA |
| | | | TGAAAGTGGTTTCGAACTTCCTGGAAGTGGGCGAATA |
| | | | CAACGCCATCGCCGCTACCGGGATGCTGTGGGATTCC |
| | | | GCCCAGGCGGCGGAACAGAAGAACGGCTATCTGGCCC |
| | | | AGGTGTTGGATGAAATCCGCCACACCCACCAGTGTGC |
| | | | CTACGTCAACTACTACTTCGCGAAGAACGGCCAGGAC |
| | | | CCGGCCGGTCACAACGATGCTCGCCGCACCCGTACCA |
| | | | TCGGTCCGCTGTGGAAGGGCATGAAGCGCGTGTTTTC |
| | | | CGACGGCTTCATTTCCGGCGACGCCGTGGAATGCTCC |
| | | | CTCAACCTGCAGCTGGTGGGTGAGGCCTGCTTCACCA |
| | | | ATCCGCTGATCGTCGCAGTGACCGAATGGGCTGCCGC |
| | | | CAACGGCGATGAAATCACCCCGACGGTGTTCCTGTCG |
| | | | ATCGAGACCGACGAACTGCGCCACATGGCCAACGGTT |
| | | | ACCAGACCGTCGTTTCCATCGCCAACGATCCGGCTTC |
| | | | CGCCAAGTATCTCAACACGGACCTGAACAACGCCTTC |
| | | | TGGACCCAGCAGAAGTACTTCACGCCGGTGTTGGGCA |
| | | | TGCTGTTCGAGTATGGCTCCAAGTTCAAGGTCGAGCC |
| | | | GTGGGTCAAGACGTGGAACCGCTGGGTGTACGAGGAC |
| | | | TGGGGCGGCATCTGGATCGGCCGTCTGGGCAAGTACG |
| | | | GGGTGGAGTCGCCGCGCAGCCTCAAGGACGCCAAGCA |
| | | | GGACGCTTACTGGGCTCACCACGACCTGTATCTGCTG |
| | | | GCTTATGCGCTGTGGCCGGGCGGCTTCTTCCGTCTGG |
| | | | CGCTGCCGGATCAGGAAGAAATGGAGTGGTTCGAGGC |
| | | | CAACTACCCCGGCTGGTACGACCACTACGGCAAGATC |
| | | | TACGAGGAATGGCGCGCCCGCGGTTGCGAGGATCCGT |
| | | | CCTCGGGCTTCATCCCGCTGATGTGGTTCATCGAAAA |
| | | | CAACCATCCCATCTACATCGATCGCGTGTCGCAAGTG |
| | | | CCGTTCTGCCCGAGCTTGGCCAAGGGCGCCAGCACCC |
| | | | TGCGCGTGCACGAGTACAACGGCCAGATGCACACCTT |
| | | | CAGCGACCAGTGGGGCGAGCGCATGTGGCTGGCCGAG |
| | | | CCGGAGCGCTACGAGTGCCAGAACATCTTCGAACAGT |
| | | | ACGAAGGACGCGAACTGTCGGAAGTGATCGCCGAACT |
| | | | GCACGGGCTGCGCAGTGATGGCAAGACCCTGATCGCC |
| | | | CAGCCGCATGTCCGTGGCGACAAGCTGTGGACGTTGG |
| | | | ACGATATCAAACGCCTGAACTGCGTCTTCAAGAACCC |
| | | | GGTGAAGGCATTCAATTGAAACGGGTGTCGGGCTCCG |
| | | | TCACAGGGCGGGGCCCGACGCACGATCGTTCGATCAA |
| | | | CCTCAAACCAAAAAGGAACATCGATATGAGCATGTTA |
| | | | GGAGAAAGACGCCGCGGTCTGACCGATCCGGAAATGG |
| | | | CGGCCGTCATTTTGAAGGCGCTTCCTGAAGCTCCGCT |
| | | | GGACGGCAACAACAAGATGGGTTATTTCGTCACCCCC |
| | | | CGCTGGAAACGCTTGACGGAATATGAAGCCCTGACCG |
| | | | TTTATGCGCAGCCCAACGCCGACTGGATCGCCGGCGG |
| | | | CCTGGACTGGGGCGACTGGACCCAGAAATTCCACGGC |
| | | | GGCCGCCCTTCCTGGGGCAACGAGACCACGGAGCTGC |
| | | | GCACCGTCGACTGGTTCAAGCACCGTGACCCGCTCCG |
| | | | CCGTTGGCATGCGCCGTACGTCAAGGACAAGGCCGAG |
| | | | GAATGGCGCTACACCGACCGCTTCCTGCAGGGTTACT |
| | | | CCGCCGACGGTCAGATCCGGGCGATGAACCCGACCTG |
| | | | GCGGGACGAGTTCATCAACCGGTATTGGGGCGCCTTC |
| | | | CTGTTCAACGAATACGGATTGTTCAACGCTCATTCGC |
| | | | AGGGCGCCCGGGAGGCGCTGTCGGACGTAACCCGCGT |
| | | | CAGCCTGGCTTTCTGGGGCTTCGACAAGATCGACATC |
| | | | GCCCAGATGATCCAACTCGAACGGGGTTTCCTCGCCA |
| | | | AGATCGTACCCGGTTTCGACGAGTCCACAGCGGTGCC |
| | | | GAAGGCCGAATGGACGAACGGGGAGGTCTACAAGAGC |
| | | | GCCCGTCTGGCCGTGGAAGGGCTGTGGCAGGAGGTGT |
| | | | TCGACTGGAACGAGAGCGCTTTCTCGGTGCACGCCGT |
| | | | CTATGACGCGCTGTTCGGTCAGTTCGTCCGCCGCGAG |
| | | | TTCTTTCAGCGGCTGGCTCCCCGCTTCGGCGACAATC |
| | | | TGACGCCATTCTTCATCAACCAGGCCCAGACATACTT |
| | | | CCAGATCGCCAAGCAGGGCGTACAGGATCTGTATTAC |
| | | | AACTGTCTGGGTGACGATCCGGAGTTCAGCGATTACA |
| | | | ACCGTACCGTGATGCGCAACTGGACCGGCAAGTGGCT |
| | | | GGAGCCCACGATCGCCGCTCTGCGCGACTTCATGGGG |
| | | | CTGTTTGCGAAGCTGCCGGCGGGCACCACTGACAAGG |
| | | | AAGAAATCACCGCGTCCCTGTACCGGGTGGTCGACGA |
| | | | CTGGATCGAGGACTACGCCAGCAGGATCGACTTCAAG |
| | | | GCGGACCGCGATCAGATCGTTAAAGCGGTTCTGGCAG |
| | | | GATTGAAATAATAGAGGAACTATTACGATGAGCGTAA |
| | | | ACAGCAACGCATACGACGCCGGCATCATGGGCCTGAA |
| | | | AGGCAAGGACTTCGCCGATCAGTTCTTTGCCGACGAA |
| | | | AACCAAGTGGTCCATGAAAGCGACACGGTCGTTCTGG |
| | | | TCCTCAAGAAGTCGGACGAGATCAATACCTTTATCGA |
| | | | GGAGATCCTTCTGACGGACTACAAGAAGAACGTCAAT |
| | | | CCGACGGTAAACGTGGAAGACCGCGCGGGTTACTGGT |
| | | | GGATCAAGGCCAACGGCAAGATCGAGGTCGATTGCGA |
| | | | CGAGATTTCCGAGCTGTTGGGGCGGCAGTTCAACGTC |
| | | | TACGACTTCCTCGTCGACGTTTCCTCCACCATCGGCC |
| | | | GGGCCTATACCCTGGGCAACAAGTTCACCATTACCAG |
| | | | TGAGCTGATGGGCCTGGACCGCAAGCTCGAAGACTCT |
| | | | CACGCTTAAGGAGAATGACATGGCGAAACTGGGTATA |
| | | | CACAGCAACGACACCCGCGACGCCTGGGTGAACAAGA |
| | | | TCGCGCAGCTCAACACCCTGGAAAAAGCGGCCGAGAT |
| | | | GCTGAAGCAGTTCCGGATGGACCACACCACGCCGTTC |
| | | | CGCAACAGCTACGAACTGGACAACGACTACCTCTGGA |
| | | | TCGAGGCCAAGCTCGAAGAGAAGGTCGCCGTCCTCAA |
| | | | GGCAGAAGCCTTCAACGAGGTGGACTTCCGTCATAAG |
| | | | ACCGCTTTCGGCGAGGATGCCAAGTCCGTTCTGGACG |
| | | | GCACCGTCGCGAAGATGAACGCGGCCAAGGACAAGTG |
| | | | GGAGGCGGAGAAGATCCATATCGGTTTCCGCCAGGCC |
| | | | TACAAGCCGCCGATCATGCCGGTGAACTATTTCCTGG |
| | | | ACGGCGAGCGTCAGTTGGGGACCCGGCTGATGGAACT |
| | | | GCGCAACCTCAACTACTACGACACGCCGCTGGAAGAA |
| | | | CTGCGCAAACAGCGCGGTGTGCGGGTGGTGCATCTGC |
| | | | AGTCGCCGCACTGAAGGGAGGAAGTCTCGCCCTGGAC |
| | | | GCGACGGCATCGCCGTGAAGTCCAGGGGGCAGGGATG |
| | | | CCGTTCCGGGCCGGCAGGCTGGCCCGGAATCTCTGGT |
| | | | TTTCAGGGGGCGTGCCGGTCCACGGCTCCCCCCTCCA |
| | | | TCTTTCGTAAGGAAATCACCATGGTCGAATCGGCATT |
| | | | TCAGCCATTTTCGGGCGACGCAGACGAATGGTTCGAG |
| | | | GAACCACGGCCCCAGGCCGGTTTCTTCCCTTCCGCGG |
| | | | ACTGGCATCTGCTCAAACGGGACGAGACCTACGCAGC |
| | | | CTATGCCAAGGATCTCGATTTCATGTGGCGGTGGGTC |
| | | | ATCGTCCGGGAAGAAAGGATCGTCCAGGAGGGTTGCT |
| | | | CGATCAGCCTGGAGTCGTCGATCCGCGCCGTGACGCA |
| | | | CGTACTGAATTATTTTGGTATGACCGAACAACGCGCC |
| | | | CCGGCAGAGGACCGGACCGGCGGAGTTCAACATTGAA |
| | | | CAGGTAAGTTTATGCAGCGAGTTCACACTATCACGGC |
| | | | GGTGACGGAGGATGGCGAATCGCTCCGCTTCGAATGC |
| | | | CGTTCGGACGAGGACGTCATCACCGCCGCCCTGCGCC |
| | | | AGAACATCTTTCTGATGTCGTCCTGCCGGGAGGGCGG |
| | | | CTGTGCGACCTGCAAGGCCTTGTGCAGCGAAGGGGAC |
| | | | TACGACCTCAAGGGCTGCAGCGTTCAGGCGCTGCCGC |
| | | | CGGAAGAGGAGGAGGAAGGGTTGGTGTTGTTGTGCCG |
| | | | GACCTACCCGAAGACCGACCTGGAAATCGAACTGCCC |
| | | | TATACCCATTGCCGCATCAGTTTTGGTGAGGTCGGCA |
| | | | GTTTCGAGGCGGAGGTCGTCGGCCTCAACTGGGTTTC |
| | | | GAGCAACACCGTCCAGTTTCTTTTGCAGAAGCGGCCC |
| | | | GACGAGTGCGGCAACCGTGGCGTGAAATTCGAACCCG |
| | | | GTCAGTTCATGGACCTGACCATCCCCGGCACCGATGT |
| | | | CTCCCGCTCCTACTCGCCGGCGAACCTTCCTAATCCC |
| | | | GAAGGCCGCCTGGAGTTCCTGATCCGCGTGTTACCGG |
| | | | AGGGACGGTTTTCGGACTACCTGCGCAATGACGCGCG |
| | | | TGTCGGACAGGTCCTCTCGGTCAAAGGGCCACTGGGC |
| | | | GTGTTCGGTCTCAAGGAGCGGGGCATGGCGCCGCGCT |
| | | | ATTTCGTGGCCGGCGGCACCGGGTTGGCGCCGGTGGT |
| | | | CTCGATGGTGCGGCAGATGCAGGAGTGGACCGCGCCG |
| | | | AACGAGACCCGCATCTATTTCGGTGTGAACACCGAGC |
| | | | CGGAATTGTTCTACATCGACGAGCTCAAATCCCTGGA |
| | | | ACGATCGATGCGCAATCTCACCGTGAAGGCCTGTGTC |
| | | | TGGCACCCGAGCGGGGACTGGGAAGGCGAGCAGGGCT |
| | | | CGCCCATCGATGCGTTGCGGGAAGACCTGGAGTCCTC |
| | | | CGACGCCAACCCGGACATTTATTTGTGCGGTCCGCCG |
| | | | GGCATGATCGATGCCGCCTGCGAGCTGGTACGCAGCC |
| | | | GCGGTATCCCCGGCGAACAGGTCTTCTTCGAAAAATT |
| | | | CCTGCCGTCCGGGGCGGCCTGAACCGGGGAAGTACCG |
| | | | TGACCACCGAGCAGTTCCCGCCCCAATTCCTGCGTGA |
| | | | AATGATCGAGCAGCTGGACGCCAGCATCCAGGAGCTC |
| | | | GCACGCAAGGAAAAGGGACTTGCGGCATCCCTGGGCA |
| | | | CGGGCCGGGTCGCCGAGCTCAAGGAATACTGGGACCA |
| | | | CGTGTGGACAAGCATAGCATAGCCATTTCGGTGATGT |
| | | | TATATCGCGTTGATTATTGATGCTGTTTTTAGTTTTA |
| | | | ACGGCAATTAATATATATGTTATTAATTGAATGAATT |
| | | | TTATTATTCATTATATATATGTGTAGAATCGTGCGCA |
| | | | GGAGAAATATTCACTCAGGAAGTTATTACTCAGGAAG |
| | | | CAAAGAGGATTACAGAATTATCTCATAACAAGTGTTA |
| | | | AGGGATGTTATTTCCCAGTTCTCTGTGGCATAATAAA |
| | | | CGAGTAGATGCTCATTCTATCTCTTATGTTCGCCTTA |
| | | | GTGCCTCATAAACTCCGGAATGACGCAGAGCCGTTTA |
| | | | CGGTGCTTATCGTCCACTGACAGATGTCGCTTATGCC |
| | | | TCATCAGACACCATGGACACAACG |

### EXAMPLES

### Example 1: Construction of E. Coli Strains with Integrated Methane Monooxygenase (MMO)

The MMO used in the construction of these E. coli strains is a modified version of mmoXYBZDC from M. capsulatus. The enzyme was improved for the conversion of methane to methanol by directed evolution and contains four amino acid substitutions relative to the wild type sequence. Briefly, a library of single mutations was screened for activity. Beneficial mutations were identified and recombined in random combinations averaging four mutations per plasmid in plasmids encoding both MMO and chaperones, and the plasmids screened again using methane as a substrate. The variant converting methane to methanol at the highest rate, containing amino acid substitutions of mmoX (V23G, T356G), mmoZ (R70E), mmoB (Y139S) was adopted. A mutation (N409G) in the groEL-2 (mmoG) protein chaperone was also incorporated. This plasmid was named pNH284 (SEQ ID NO: 44) and this strain was named NH848.

Using a CRISPR-based strategy as described by Bassalo, et al. (ACS Synth. Biol. 5 (7): 561-568, 2016, which is incorporated by reference herein, including any drawings) the coding sequence for the optimized MMO under the control of the arabinose promoter was inserted into the E. coli genome. The strain NH283 was constructed by the deletion of a region of DNA from the E. coli genome that contains the genes araBAD using the method of Datsenko and Wanner (See, K. Datsenko and B. Wanner, One-step inactivation of chromosomal genes in Escherichia coli K-12 using PCR Products, Proceedings of the National Academy of Sciences, Vol 97, Issue 12, p.6640-5, 2000, which is incorporated by reference herein in its entirety). In one strain, YZ55, a single copy of MMO was inserted at the IS7 site (as defined in Bassalo et al.; position 3099988). In a second strain, YZ71, two copies of MMO were inserted, one at IS7 and a second at SS9 (as defined in Bassalo et al.; position 3979535). Proper insertion was confirmed by sequencing.

### Example 2: Production of 3-Hydroxypropionate from Ethane Using an Engineered Microorganism

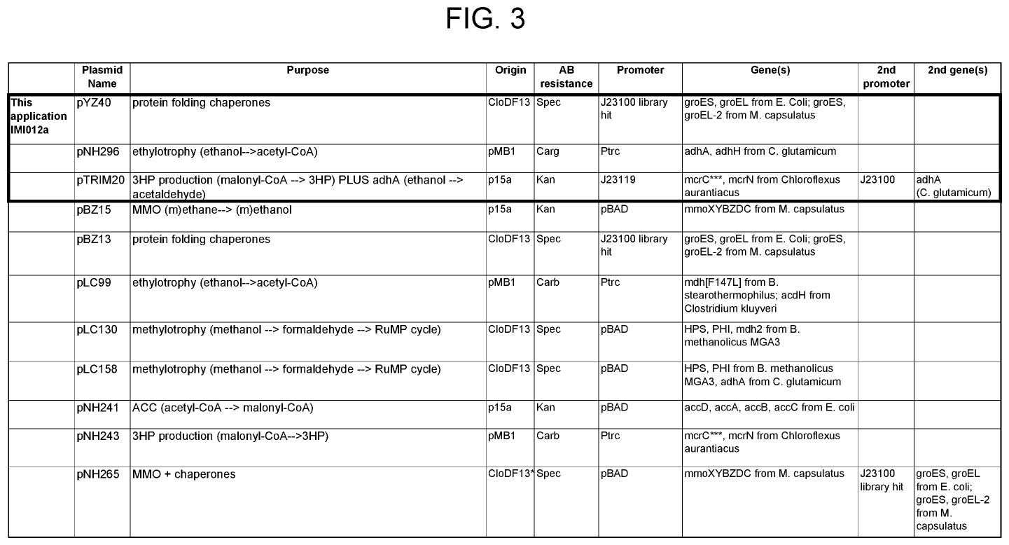

FIG. 3 shows a plasmid of comparison of the plasmids used in this invention.

3-hydroxypropionate (3HP) was produced from an ethane feedstock via the fermentation of three engineered strains of Escherichia coli. Each strain harbored three plasmids: 1) pYZ40 (SEQ ID NO: 45), conferring expression of the chaperones groEL and groES from each of E. coli and M. capsulatus; 2) pNH296 (SEQ ID NO: 46) conferring expression of adhA and adhH from C. glutamicum for the conversion of ethanol to acetyl-CoA (ethylotrophy), and 3) pTRIM20 (SEQ ID NO: 47) conferring expression of mcrC*** and mcrN from Chloroflexus aurantiacus, to enable production of 3HP from malonyl-CoA.

pYZ40 (SEQ ID NO: 45) contains a CloDF13 origin of replication, spectinomycin resistance, and the chaperone operon is under the control of the constitutive J23100 promoter. pNH296 (SEQ ID NO: 46) contains a pMB1 origin of replication, carbenicillin resistance, and the alcohol dehydrogenase operon is under the control of the IPTG-inducible pTrc promoter. pTRIM20 (SEQ ID NO: 47) contains a p15a origin of replication, kanamycin resistance, and the malonyl-CoA reductase operon is under the control of the constitutive J23119 promoter. pTRIM20 additionally contains a coding sequence for adhA under the control of the constitutive J23100 promoter.

One of the three strains for the fermentation, LC805, was derived from YZ71 (with one integrated copy of MMO) by addition of the three plasmids. Another strain, LC809, was derived from YZ55 (with two integrated copies of MMO) by addition of the three plasmids. The third strain, LC807, was derived from a strain with plasmid-borne MMO, NH848, by addition of the two plasmids pNH296 and pTRIM20. NH848 is the base strain NH283 transformed with the plasmid pNH284 (SEQ ID NO: 44).

The three strains (LC805, LC807, and LC809) were induced with arabinose and IPTG in LB for 4 hours and resuspended in BEM6. (The minimal media called BEM6 contains (in ddH₂O): 50 mM KH₂PO₄, 50 mM Na₂HPO₄*7 H₂O, 1 mM MgSO₄, 0.15% LB, 1.5625 mM glutamine, 80 μM FeSO₄, 0.1 mM CaCl₂), 1 mM IPTG, 0.1% of the 1000× metals solution, and 1 mM L-arabinose.) The 1000× metals solution contains (in ddH₂O): 50 mM FeCl₃, 20 mM CaCl₂, 10 mM MnCl₂, 10 mM ZnSO₄, 2 mM CoCl₂, 2 mM NiCl₂, 2 mM Na₂MoO₄, 2 mM Na₂SeO₃, 2 mM H₃BO₃. This cell culture was split between two serum bottles; each bottle was stoppered and injected with 90 mL of either nitrogen or ethane. The serum bottles were shaken for 96 hours at 37 C. Samples were taken and supernatant was subjected to NMR analysis. The 3HP titer was determined and the titer from the ethane condition was compared to that from the nitrogen condition either as total concentration (FIG. 1A) or normalized by starting OD₆₀₀ (FIG. 1B). This procedure was repeated using LC805 and ten-fold different starting ODs of LC809 (FIG. 1C).

LC809 (having two integrated copies of MMO) consistently produced 120-130 mg/L/OD of 3HP. LC809 shows a substantially higher production of 3HP than LC805, having a single integrated copy of MMO, as might have been expected. Surprisingly, however, LC809 also shows substantially higher production of 3HP than LC807, which had a plasmid-borne MMO.

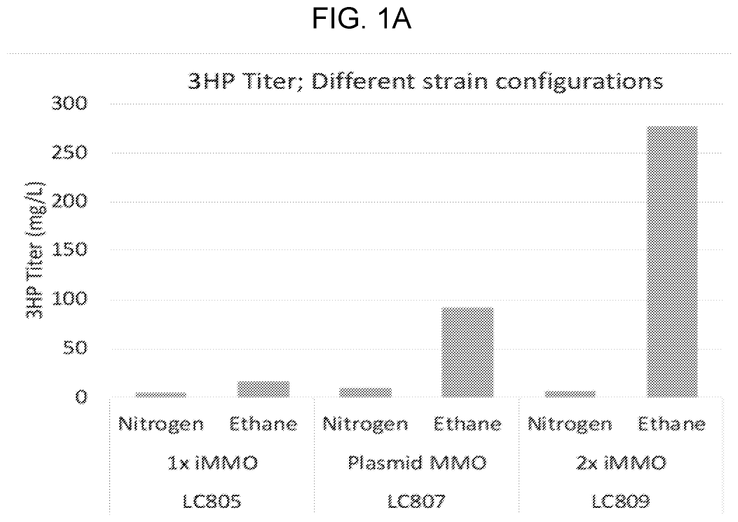

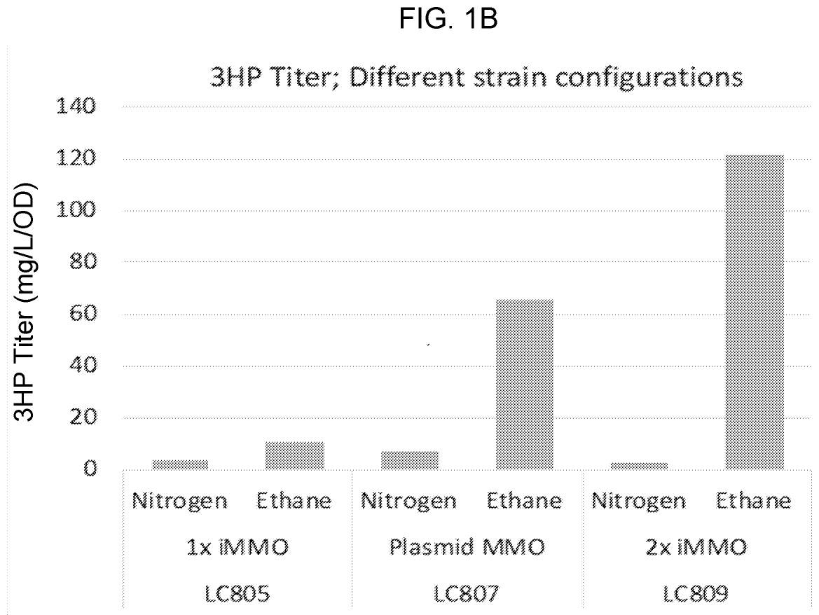

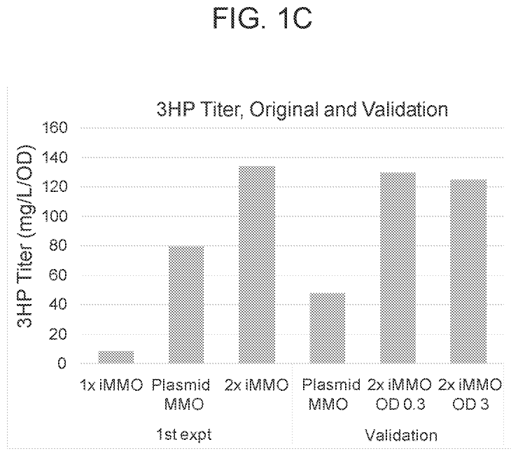

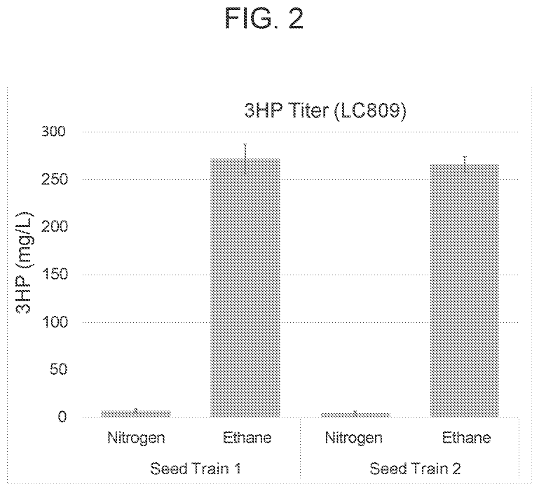

When using ethane/ethanol as a primary or sole carbon source, different biochemical pathways need to be active as opposed to when bacteria has a plentiful supply of, for example, 5-carbon sugars or 6-carbon sugars. Better growth can sometimes be obtained if ethanol is included in the seed train, to induce up-regulation of these pathways. Thus, 3HP production by LC809 was compared using a seed train of LB overnight, followed by 1:30 dilution into LB induction, followed by centrifugation and resuspension in BEM6 (starting OD˜2.3) (Seed Train 1), with the same supplemented with 0.5% ethanol (Seed Train 2). Equivalent amounts of 3HP were produced in both conditions (FIG. 2).

All publications, patents, and patent applications cited in this specification are hereby incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A synthetic microorganism comprising or consisting of at least one exogenous polynucleotide that encodes one or more polypeptides comprising or consisting of an ethane monooxygenase, an ethylotrophy pathway, and/or a malonyl-CoA-product pathway.

2. The synthetic microorganism according to claim 1, wherein the synthetic microorganism further comprises a microorganism with the at least one exogenous polynucleotide inserted or integrated into the genome of the microorganism.

3. The synthetic microorganism of claim 1, wherein the ethane monooxygenase comprises or consists of an ethane monooxygenase having an amino acid sequence that is more than about 70%, more than about 75%, more than about 80%, more than about 85%, more than about 90%, or more than about 95% identical or identical to any of SEQ ID NOs: 1-16.

4. The synthetic microorganism of claim 1, wherein the ethylotrophy pathway comprises or consists of one or more enzymes having an amino acid sequence that is more than about 70%, more than about 75%, more than about 80%, more than about 85%, more than about 90%, or more than about 95% identical or identical to any of SEQ ID NOs 22-23.

5. The synthetic microorganism of claim 1, wherein the malonyl-CoA-product pathway comprises or consists of one or more enzymes having an amino acid sequence that is more than about 70%, more than about 75%, more than about 80%, more than about 85%, more than about 90%, or more than about 95% identical or identical to any of SEQ ID NOs: 29-30.

6. The synthetic microorganism of claim 1, wherein the synthetic microorganism generates a product comprising or consisting of 3-hydroxypropionic acid.

7. The synthetic microorganism of claim 1, wherein the synthetic microorganism generates a product comprising or consisting of a polymer of 3-hydroxypropionic acid.

8. The synthetic microorganism of claim 1, wherein the synthetic microorganism generates one or more products comprising or consisting of fatty acids or fatty acid derivatives.

9. The synthetic microorganism of claim 1, wherein the synthetic microorganism generates a product comprising or consisting of polymer(s) of 3-hydroxy-fatty acids.

10. The synthetic microorganism of claim 1, wherein the synthetic microorganism generates a product comprising or consisting of malonic acid.

11. The synthetic microorganism of claim 1, further comprising one or more exogenous polynucleotides which overexpress acetyl-CoA carboxylase.

12. The synthetic microorganism of claim 1, wherein the microorganism is derived from Escherichia coli.

13. A method of culturing the synthetic microorganism of claim 1 for a suitable period of time and under conditions sufficient for conversion of ethane into the malonyl-CoA-derived product.


SEQUENCE LISTING (49 sequences)

SEQ ID NO: 1 - protein, 527 aa, Methylococcus capsulatus
MALSTATKAATDALAANRAPTSGNAQEVHRWLQSFNWDFKNNRTKYATKYKMANETKEQFKLIAKEYARMEAVKDERQFGSLQDALTRLNAGVRVHPKWNETMKVVSNFLEVGEYNAIAATGMLWDSAQAAEQKNGYLAQVLDEIRHTHQCAYVNYYFAKNGQDPAGHNDARRTRTIGPLWKGMKRVFSDGFISGDAVECSLNLQLVGEACFTNPLIVAVTEWAAANGDEITPTVFLSIETDELRHMANGYQTVVSIANDPASAKYLNTDLNNAFWTQQKYFTPVLGMLFEYGSKFKVEPWVKTWNRWVYEDWGGIWIGRLGKYGVESPRSLKDAKQDAYWAHHDLYLLAYALWPGGFFRLALPDQEEMEWFEANYPGWYDHYGKIYEEWRARGCEDPSSGFIPLMWFIENNHPIYIDRVSQVPFCPSLAKGASTLRVHEYNGQMHTFSDQWGERMWLAEPERYECQNIFEQYEGRELSEVIAELHGLRSDGKTLIAQPHVRGDKLWTLDDIKRLNCVFKNPVKAFN

SEQ ID NO: 2 - protein, 389 aa, Methylococcus capsulatus
MSMLGERRRGLTDPEMAAVILKALPEAPLDGNNKMGYFVTPRWKRLTEYEALTVYAQPNADWIAGGLDWGDWTQKFHGGRPSWGNETTELRTVDWFKHRDPLRRWHAPYVKDKAEEWRYTDRFLQGYSADGQIRAMNPTWRDEFINRYWGAFLFNEYGLFNAHSQGAREALSDVTRVSLAFWGFDKIDIAQMIQLERGFLAKIVPGFDESTAVPKAEWTNGEVYKSARLAVEGLWQEVFDWNESAFSVHAVYDALFGQFVRREFFQRLAPRFGDNLTPFFINQAQTYFQIAKQGVQDLYYNCLGDDPEFSDYNRTVMRNWTGKWLEPTIAALRDFMGLFAKLPAGTTDKEEITASLYRVVDDWIEDYASRIDFKADRDQIVKAVLAGLK

SEQ ID NO: 3 - protein, 141 aa, Methylococcus capsulatus
MSVNSNAYDAGIMGLKGKDFADQFFADENQVVHESDTVVLVLKKSDEINTFIEEILLTDYKKNVNPTVNVEDRAGYWWIKANGKIEVDCDEISELLGRQFNVYDFLVDVSSTIGRAYTLGNKFTITSELMGLDRKLEDSHA

SEQ ID NO: 4 - protein, 170 aa, Methylococcus capsulatus
MAKLGIHSNDTRDAWVNKIAQLNTLEKAAEMLKQFRMDHTTPFRNSYELDNDYLWIEAKLEEKVAVLKAEAFNEVDFRHKTAFGEDAKSVLDGTVAKMNAAKDKWEAEKIHIGFRQAYKPPIMPVNYFLDGERQLGTRLMELRNLNYYDTPLEELRKQRGVRVVHLQSPH

SEQ ID NO: 5 - protein, 103 aa, Methylococcus capsulatus
MVESAFQPFSGDADEWFEEPRPQAGFFPSADWHLLKRDETYAAYAKDLDFMWRWVIVREERIVQEGCSISLESSIRAVTHVLNYFGMTEQRAPAEDRTGGVQH

SEQ ID NO: 6 - protein, 348 aa, Methylococcus capsulatus
MQRVHTITAVTEDGESLRFECRSDEDVITAALRQNIFLMSSCREGGCATCKALCSEGDYDLKGCSVQALPPEEEEEGLVLLCRTYPKTDLEIELPYTHCRISFGEVGSFEAEVVGLNWVSSNTVQFLLQKRPDECGNRGVKFEPGQFMDLTIPGTDVSRSYSPANLPNPEGRLEFLIRVLPEGRFSDYLRNDARVGQVLSVKGPLGVFGLKERGMAPRYFVAGGTGLAPVVSMVRQMQEWTAPNETRIYFGVNTEPELFYIDELKSLERSMRNLTVKACVWHPSGDWEGEQGSPIDALREDLESSDANPDIYLCGPPGMIDAACELVRSRGIPGEQVFFEKFLPSGAA

SEQ ID NO: 7 - protein, 260 aa, Methylococcus capsulatus
MAATTIGGAAAAEAPLLDKKWLTFALAIYTVFYLWVRWYEGVYGWSAGLDSFAPEFETYWMNFLYTEIVLEIVTASILWGYLWKTRDRNLAALTPREELRRNFTHLVWLVAYAWAIYWGASYFTEQDGTWHQTIVRDTDFTPSHIIEFYLSYPIYIITGFAAFIYAKTRLPFFAKGISLPYLVLVVGPFMILPNVGLNEWGHTFWFMEELFVAPLHYGFVIFGWLALAVMGTLTQTFYSFAQGGLGQSLCEAVDEGLIAK

SEQ ID NO: 8 - protein, 420 aa, Mycobacterium sp.
MTEMTVAASDATNAAYGMALEDIDVSNPVLFRDNTWHPYFKRLREEDPVHYCKSSMFGPYWSVTKYRDIMAVETNPKVFSSEAKSGGITIMDDNAAASLPMFIAMDPPKHDVQRKTVSPIVAPENLATMESVIRQRTADLLDGLPINEEFDWVHRVSIELTTKMLATLFDFPWDDRAKLTRWSDVTTALPGGGIIDSEEQRMAELMECATYFTELWNQRVNAEPKNDLISMMAHSESTRHMAPEEYLGNIVLLIVGGNDTTRNSMTGGVLALNEFPDEYRKLSANPALISSMVSEIIRWQTPLSHMRRTALEDIEFGGKHIRQGDKVVMWYVSGNRDPEAIDNPDTFIIDRAKPRQHLSFGFGIHRCVGNRLAELQLNILWEEILKRWPDPLQIQVLQEPTRVLSPFVKGYESLPVRINA

SEQ ID NO: 9 - protein, 415 aa, Pseudomonas putida
MTTETIQSNANLAPLPPHVPEHLVFDFDMYNPSNLSAGVQEAWAVLQESNVPDLVWTRCNGGHWIATRGQLIREAYEDYRHFSSECPFIPREAGEAYDFIPTSMDPPEQRQFRALANQVVGMPVVDKLENRIQELACSLIESLRPQGQCNFTEDYAEPFPIRIFMLLAGLPEEDIPHLKYLTDQMTRPDGSMTFAEAKEALYDYLIPIIEQRRQKPGTDAISIVANGQVNGRPITSDEAKRMCGLLLVGGLDTVVNFLSFSMEFLAKSPEHRQELIERPERIPAACEELLRRFSLVADGRILTSDYEFHGVQLKKGDQILLPQMLSGLDERENACPMHVDFSRQKVSHTTFGHGSHLCLGQHLARREIIVTLKEWLTRIPDFSIAPGAQIQHKSGIVSGVQALPLVWDPATTKAV

SEQ ID NO: 10 - protein, 415 aa, Pseudomonas putida
MTTETIQSNANLAPLPPHVPEHLVFDFDMYNPSNLSAGVQEAWAVLQESNVPDLVWTRCNGGHWIATRGQLIREAYEDYRHFSSECPWIPREAGEAFDFIPLSMDPPEQRQFRALANQVVGMPVVDKLENRIQELACSLIESLRPQGQCNFTEDYAEPFPIRIFMLLAGLPEEDIPHLKYLTDQMMRPDGSMTFAEAKEALYDYLIPIIEQRRQKPGTDAISIVANGQVNGRPITSDEAKRMCGMLLLAGLDTVVNFLSFSMEFLAKSPEHRQELIERPERIPAACEELLRRFSMVADGRILTSDYEFHGVQLKKGDQILLPQMLSGLDERENACPMHVDFSRQKVSHTTFGHGSHLCPGQHLARREIIVTLKEWLTRIPDFSIAPGAQIQHKSGIVSGVQALPLVWDPATTKAV

SEQ ID NO: 11 - protein, 401 aa, Pseudomonas putida
MLEKHRVLDSAPEYVDKKKYLWILSTLWPATPMIGIWLANETGWGIFYGLVLLVWYGALPLLDAMFGEDFNNPPEEVVPKLEKERYYRVLTYLTVPMHYAALIVSAWWVGTQPMSWLEIGALALSLGIVNGLALNTGHELGHKKETFDRWMAKIVLAVVGYGHFFIEHNKGHHRDVATPMDPATSRMGESIYKFSIREIPGAFIRAWGLEEQRLSRRGQSVWSFDNEILQPMIITVILYAVLLALFGPKMLVFLPIQMAFGWWQLTSANYIEHYGLLRQKMEDGRYEHQKPHHSWNSNHIVSNLVLFHLQRHSDHHAHPTRSYQSLRDFPGLPALPTGYPGAFLMAMIPQWFRSVMDPKVVDWAGGDLNKIQIDDSMRETYLKKFGTSSAGHSSSTSAVAS

SEQ ID NO: 12 - protein, 545 aa, Gordonia sp.
MSRQSLTKAHAKITELSWEPTFATPATRFGTDYTFEKAPKKDPLKQIMRSYFPMEEEKDNRVYGAMDGAIRGNMFRQVQERWLEWQKLFLSIIPFPEISAARAMPMAIDAVPNPEIHNGLAVQMIDEVRHSTIQMNLKKLYMNNYIDPAGFDITEKAFANNYAGTIGRQFGEGFITGDAITAANIYLTVVAETAFTNTLFVAMPDEAAANGDYLLPTVFHSVQSDESRHISNGYSILLMALADERNRPLLERDLRYAWWNNHCVVDAAIGTFIEYGTKDRRKDRESYAEMWRRWIYDDYYRSYLLPLEKYGLTIPHDLVEEAWNRIVDKHYVHEVARFFATGWPVNYWRIDAMTDTDFEWFEEKYPGWYNKFGKWWENYNRLAYPGKNKPIAFEDVDYEYPHRCWTCMVPCLIREDMVTDKVDGQWRTYCSETCAWTDKVAFRPEYEGRPTPNMGRLTGFREWETLHHGKDLADIITDLGYVRDDGKTLIPQPHLDLDPKKMWTLDDVRGIPFGSPNVALNEMSDDEREAHIAAYMANKNGAVTV

SEQ ID NO: 13 - protein, 549 aa, Pseudonocardia sp.
MSRQSMSKAHKKITELSWEPTFATPAKRFGTDYTFDNAPKKDPLKQILRSYFPMEEEKDSRVFGAMDGAIRGNMFRQVQERWMEWQKLFLSIIPFPEISAARAMPMAIDAVPNPEIHNGLAVQMIDEVRHSTIQMNLKRLYMNYYIDPAGFDMTEKAFANNYAGTIGRQFGEGFITGDAITAANIYLTVVAETAFTNTLFVAMPSEAAANGDYLLPTVFHSVQSDESRHISNGYSILLMALSDEDNRQLLERDLRYAWWNNHRVVDAAIGTFIEYGTKDRRKDRESYAEMWRRWIYDDYYRAYLIPLEKYGLVIPHDLIEESWKQIWEKGYVHEVAQFFATGWLANYWRIDSMTDEDFEWFEYKYPGWYDKYGKWWENYNRLSKPNGHNPIVFEDVDYVYPARCWTCMSPCWSVRTLVTAEVDGQHRTYCHEVCRWTDVRGFPSDVPGRETPNMGRLVGKREWETLYHGWNWADVVSDMGFVRDDGKTMTPKPHLDLDPKKMWTLDHMRRCPPLQSPNVLFNEMSDAERAAYVADYNKQGPAGRPAPQS

SEQ ID NO: 14 - protein, 531 aa, Mycobacterium sp.
MTSTLPGKVSGHPSAPHASVSGQEVHSWLMDLGWDSDTIRGKYPTKYKFDPNAREQFKLVARDYGRMEGEKDDRQYGSLLDSLARLKAPTRVEPRWAEVMKLLAGALELGEYNAIAGSAVLADTTRSPELRNGYLMQVEDEVRHTTQTHYLAKYYAGQYYDPAGFTDMRKWRYINPLFPPTMQAFGENFCAGDPVFASLNLQLVAEACFTNPLIVAMTEWSAANGDEITPTIFLSIQSDEMRHMANGYQTIVSVAHDADNMQYLQTDLENAFWLQHRFATPIVGAGFEYGAVNKLEPWAKVWDRWVYEDWGGIWLGRLEKFGVKSPANLADAKRQAYWGHHYTYAVAYAVWPLLGFRMDPPNARDMEWFENNYPGWHSEVGHMWDSWREMGLADPANHTLPGQLVSDAKVPIYFCRVCNFPVIIPTLTGALDDVRILELNGRKHPLCSSWCERMFLKEPERYQGENLWEKFDGWNIADVVHAAGAVRSDGKTLLAQPHLNSERMWTLDDLRACHAVIRDPLKTGGVWLETV

SEQ ID NO: 15 - protein, 530 aa, Thauera butanivorans
MSANMAVKQALKANPVPSSVDPQEVHKWLQDFTWDFKGKTAKYPTKYEMDVNTREQFKLTAKEYARMESIKEERQYGTLLDGLDRLDAGNKVHPKWGEVMKLVSNFLETGEYGAIAGSALLWDTAQSPEQRNGYLAQVIDEIRHVNQTAYVNYYYGKHYYDPAGHTNMRQLRAINPLYPGVKRAFGEGFLAGDAVESSINLQLVGEACFTNPLIVSLTEWAAANGDEITPTVFLSIETDELRHMANGYQTIVSIMNNPETMKYLQTDLDNAFWTQHKFLTPFVGVALEYGSKYKVEPWAKSWNRWVYEDWAGIWLGRLQQFGVKTPKCLPDAKKDAVWAHHDLALLALALWPLTGIRMELPDSLAMEWFEANYPGWYNHYGKIYEEWRAAGFEDPKSGFCGALWLMERGHGIFVDHASGLPFCPSLAKSSIKPRFTEYNGKRYAFAEPYGERQWLLEPERYEFQNFFEQFEGWELSDLVKAAGGVRSDGKTLIAQPHLRDTDMWTLDDLKRINLTIPDPMKILNWQPVAQ

SEQ ID NO: 16 - protein, 276 aa, Methylacidiphilum infernorum
MAQATTQTVSISIPERSAFSWKNLWIALAIITAFELAVNVYERTFAIAKGLDYFTPQYQTYWMSILYTELILEPTTLIALCSWLWVTRDRAMENLAPAEELKRYWNLGLFVVVYTVLLYWGASYYTEQDGTWHQTVIRDTDFTPSHIIEFYQSYPIYIVAGVGSMVYAMTRLPQYAKAFSVPYAVLVGSPLMIFPNVGLNEFGHTRWFMEELFVAPLHWGFVMFGWGALAILGTWLQICPRVVELIKQVYYGKPATAPAVVLNDPEKAADPALCEI

SEQ ID NO: 17-21 - skipped (5 entries)

SEQ ID NO: 22 - protein, 345 aa, Corynebacterium glutamicum
MTTAAPQEFTAAVVEKFGHDVTVKDIDLPKPGPHQALVKVLTSGICHTDLHALEGDWPVKPEPPFVPGHEGVGEVVELGPGEHDVKVGDIVGNAWLWSACGTCEYCITGRETQCNEAEYGGYTQNGSFGQYMLVDTRYAARIPDGVDYLEAAPILCAGVTVYKALKVSETRPGQFMVISGVGGLGHIAVQYAAAMGMRVIAVDIADDKLELARKHGAEFTVNARNEDSGEAVQKYTNGGAHGVLVTAVHEAAFGQALDMARRAGTIVFNGLPPGEFPASVFNIVFKGLTIRGSLVGTRQDLAEALDFFARGLIKPTVSECSLDEVNGVLDRMRNGKIDGRVAIRF

SEQ ID NO: 23 - protein, 506 aa, Corynebacterium glutamicum
MTVYANPGTEGSIVNYEKRYENYIGGKWVPPVEGQYLENISPVTGEVFCEVARGTAADVELALDAAHAAADAWGKTSVAERALILHRIADRMEEHLEEIAVAETWENGKAVRETLAADIPLAIDHFRYFAGAIRAQEDRSSQIDHNTVAYHFNEPIGVVGQIIPWNFPILMATWKLAPALAAGNAIVMKPAEQTPASILYLINIIGDLIPEGVLNIVNGLGGEAGAALSGSNRIGKIAFTGSTEVGKLINRAASDKIIPVTLELGGKSPSIFFSDVLSQDDAFAEKAVEGFAMFALNQGEVCTCPSRALVHESIADEFLELGVKRVQNIKLGNPLDTETMMGAQASQEQMDKISSYLKIGPEEGAQTLTGGKVNKVDGMENGYYIEPTVFRGTNDMRIFREEIFGPVLSVATFSDFDEAIRIANDTNYGLGAGVWSRDQNTIYRAGRAIQAGRVWVNQYHNYPAHSAFGGYKESGIGRENHLMMLNHYQQTKNLLVSYDPNPTGLF

SEQ ID NO: 24-28 - skipped (5 entries)

SEQ ID NO: 29 - protein, 671 aa, Chloroflexus aurantiacus
MSATTGARSASVGWAESLIGLHLGKVALITGGSAGIGGQIGRLLALSGARVMLAARDRHKLEQMQAMIQSELAEVGYTDVEDRVHIAPGCDVSSEAQLADLVERTLSAFGTVDYLINNAGIAGVEEMVIDMPVEGWRHTLFANLISNYSLMRKLAPLMKKQGSGYILNVSSYFGGEKDAAIPYPNRADYAVSKAGQRAMAEVFARFLGPEIQINAIAPGPVEGDRLRGTGERPGLFARRARLILENKRLNELHAALIAAARTDERSMHELVELLLPNDVAALEQNPAAPTALRELARRFRSEGDPAASSSSALLNRSIAAKLLARLHNGGYVLPADIFANLPNPPDPFFTRAQIDREARKVRDGIMGMLYLQRMPTEFDVAMATVYYLADRVVSGETFHPSGGLRYERTPTGGELFGLPSPERLAELVGSTVYLIGEHLTEHLNLLARAYLERYGARQVVMIVETETGAETMRRLLHDHVEAGRLMTIVAGDQIEAAIDQAITRYGRPGPVVCTPFRPLPTVPLVGRKDSDWSTVLSEAEFAELCEHQLTHHFRVARWIALSDGARLALVTPETTATSTTEQFALANFIKTTLHAFTATIGVESERTAQRILINQVDLTRRARAEEPRDPHERQQELERFIEAVLLVTAPLPPEADTRYAGRIHRGRAITV

SEQ ID NO: 30 - protein, 550 aa, Chloroflexus aurantiacus
MSGTGRLAGKIALITGGAGNIGSELTRRFLAEGATVIISGRNRAKLTALAERMQAEAGVPAKRIDLEVMDGSDPVAVRAGIEAIVARHGQIDILVNNAGSAGAQRRLAEIPLTEAELGPGAEETLHASIANLLGMGWHLMRIAAPHMPVGSAVINVSTIFSRAEYYGRIPYVTPKAALNALSQLAARELGARGIRVNTIFPGPIESDRIRTVFQRMDQLKGRPEGDTAHHFLNTMRLCRANDQGALERRFPSVGDVADAAVFLASAESAALSGETIEVTHGMELPACSETSLLARTDLRTIDASGRTTLICAGDQIEEVMALTGMLRTCGSEVIIGFRSAAALAQFEQAVNESRRLAGADFTPPIALPLDPRDPATIDAVFDWGAGENTGGIHAAVILPATSHEPAPCVIEVDDERVLNFLADEITGTIVIASRLARYWQSQRLTPGARARGPRVIFLSNGADQNGNVYGRIQSAAIGQLIRVWRHEAELDYQRASAAGDHVLPPVWANQIVRFANRSLEGLEFACAWTAQLLHSQRHINEITLNIPANI

SEQ ID NO: 31-35 - skipped (5 entries)

SEQ ID NO: 36 - protein, 97 aa, Escherichia coli [sequence omitted]

SEQ ID NO: 37 - protein, 542 aa, Escherichia coli
MAAKDVKFGNDARVKMLRGVNVLADAVKVTLGPKGRNVVLDKSFGAPTITKDGVSVAREIELEDKFENMGAQMVKEVASKANDAAGDGTTTATVLAQAIITEGLKAVAAGMNPMDLKRGIDKAVTAAVEELKALSVPCSDSKAIAQVGTISANSDETVGKLIAEAMDKVGKEGVITVEDGTGLQDELDVVEGMQFDRGYLSPYFINKPETGAVELESPFILLADKKISNIREMLPVLEAVAKAGKPLLIIAEDVEGEALATLVVNTMRGIVKVAAVKAPGFGDRRKAMLQDIATLTGGTVISEEIGMELEKATLEDLGQAKRVVINKDTTTIIDGVGEEAAIQGRVAQIRQQIEEATSDYDREKLQERVAKLAGGVAVIKVGAATEVEMKEKKARVEDALHATRAAVEEGVVAGGGVALIRVASKLADLRGQNEDQNVGIKVALRAMEAPLRQIVLNCGEEPSVVANTVKGGDGNYGYNAATEEYGNMIDMGILDPTKVTRSALQYAASVAGLMITTECMVTDLPKNDAADLGAAGGMGGMM

SEQ ID NO: 38 - protein, 96 aa, Methylococcus capsulatus
VKIRPLHDRVIIKRLEEERTSAGGIVIPDSAAEKPMRGEILAVGNGKVLDNGEVRALQVKVGDKVLFGKYAGTEVKVDGEDVVVMREDDILAVLES

SEQ ID NO: 39 - protein, 559 aa, Methylococcus capsulatus
MAKEVVYRGSARQRMMQGIEILARAAIPTLGATGPSVMIQHRADGLPPISTRDGVTVANSIVLKDRVANLGARLLRDVAGTMSREAGDGTTTAIVLARHIAREMFKSLAVGADPIALKRGIDRAVARVSEDIGARAWRGDKESVILGVAAVATKGEPGVGRLLLEALDAVGVHGAVSIELGQRREDLLDVVDGYRWEKGYLSPYFVTDRARELAELEDVYLLMTDREVVDFIDLVPLLEAVTEAGGSLLIAADRVHEKALAGLLLNHVRGVFKAVAVTAPGFGDKRPNRLLDLAALTGGRAVLEAQGDRLDRVTLADLGRVRRAVVSADDTALLGIPGTEASRARLEGLRLEAEQYRALKPGQGSATGRLHELEEIEARIVGLSGKSAVYRVGGVTDVEMKERMVRIEGAYRSVVSALEEGVLPGGGVGFLGSMPVLAELEARDADEARGIGIVRSALTEPLRIIGENSGLSGEAVVAKVMDHANPGWGYDQESGSFCDLHARGIWDAAKVLRLALEKAASVAGTFLTTEAVVLEIPDTDAFAGFSAEWAAATREDPRV

SEQ ID NO: 40-43 - skipped (4 entries)

SEQ ID NO: 44 - DNA, 13237 bp, Artificial Sequence
  Description of Artificial Sequence: Synthetic polynucleotide
ttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggctggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctcccaatgtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttttgacggctagctcagtcctagggataatgctagcaccagcctcgagggaaaccacgtaagctccggcgtttaaacacccataacagatacggactttctcaaaggagagttatcagtgaaaatccgcccgttacatgaccgtgtcatcatcaaacgcttggaagaagagcgtacctcggcgggcgggattgtcattccagatagcgcagctgaaaaaccgatgcgtggtgaaatcctggcagtgggcaatggaaaagtgcttgataatggagaggtacgtgctttacaggtgaaagtgggtgataaagtgctctttgggaaatacgcgggtacggaggttaaagtagatggggaagatgttgttgtcatgcgtgaagatgacattctggctgtgttagaatcttaatccgcgcacgacactgaacatacgaatttaaggaataaagataatggcgaaagaagttgtgtatcgtggtagtgcgcgccagcgtatgatgcagggtattgaaattctcgctcgcgccgctattccaacgctgggggcaaccggcccgagcgtcatgattcaacatcgcgccgatggtctgccacccatttctacacgcgatggcgttaccgtagcgaattctattgttttaaaagaccgtgtcgcgaacctgggtgcccgcctgctgcgcgacgtagccggtacaatgagccgtgaagccggcgacggcacgacgactgcgatcgtattggcccgccacatcgcccgtgagatgtttaaatcgctggccgtgggtgcagatccgatcgcgctgaaacgtggtatcgatcgcgccgttgctcgtgtgtccgaagatattggggcgcgtgcgtggcgtggcgataaagaaagcgtgatcctgggtgtcgctgctgtggcgacgaaaggcgaaccgggcgttggccgtctgctgctggaggctctcgatgcagtgggtgttcacggtgccgtttctatcgaactgggccaacgtcgtgaagatctgctggacgtcgtcgatggctatcgctgggaaaaaggttatttatctccctactttgtcacggaccgtgcccgcgaactcgcggaactggaggatgtctacctgctcatgaccgaccgcgaagtggttgacttcatcgaccttgtacctctgctggaggccgtgacggaagcaggaggctccctgctgattgccgcggatcgtgtgcacgaaaaggccttagcggggctgcttctgaatcacgtgcgcggtgtcttcaaggccgtggccgtaaccgctccgggttttggcgacaaacgcccgaaccgtttacttgacctggccgcgttaaccggcggtcgtgccgtgctcgaagctcaaggcgaccgtctggaccgtgttaccctcgcggatctgggccgtgtgcgccgtgccgtggtgtcggcagatgataccgcgctgcttggcatcccgggcaccgaagctagccgtgcacgcctcgaaggtctgcgtttagaagcagagcagtaccgtgcgctgaaaccagggcagggttctgccaccgggcgcctgcacgaacttgaagaaattgaagcgcgcattgtgggtctgtccggaaagagcgccgtttatcgcgtcggaggtgtgaccgatgtggaaatgaaagagcgcatggttcgcatcgaaggagcttaccgttcggtggtaagtgcgctggaggaaggcgtgctccctggcggtggtgtcggctttctgggtagtatgccggtgcttgcggaattggaggcccgcgacgcagatgaagctcgcgggattgggattgtacgcagcgccttaacggagcctcttcgtattatcggcgaaaatagtggcttgagcggtgaagccgttgttgccaaagtcatggatcatgccaacccgggatggggttacgaccaggagtctggctctttttgcgacctgcatgcgcgtgggatctgggatgctgctaaagtgttacgtctcgcgttggagaaggcagcctctgttgctgggacctttctgacaaccgaagctgttgttctcgaaattccggatacagatgcgttcgcagggttcagtgcagaatgggctgccgccacgcgcgaagatccgcgcgtatgagtttaaacgcggccgcaatttgaacgcacccataacagatacggactttctcaaaggagagttatcaatgaatattcgtccattgcatgatcgcgtgatcgtcaagcgtaaagaagttgaaactaaatctgctggcggcatcgttctgaccggctctgcagcggctaaatccacccgcggcgaagtgctggctgtcggcaatggccgtatccttgaaaatggcgaagtgaagccgctggatgtgaaagttggcgacatcgttattttcaacgatggctacggtgtgaaatctgagaagatcgacaatgaagaagtgttgatcatgtccgaaagcgacattctggcaattgttgaagcgtaatccgcgcacgacactgaacatacgaatttaaggaataaagataatggcagctaaagacgtaaaattcggtaacgacgctcgtgtgaaaatgctgcgcggcgtaaacgtactggcagatgcagtgaaagttaccctcggtccaaaaggccgtaacgtagttctggataaatctttcggtgcaccgaccatcaccaaagatggtgtttccgttgctcgtgaaatcgaactggaagacaagttcgaaaatatgggtgcgcagatggtgaaagaagttgcctctaaagcaaacgacgctgcaggcgacggtaccaccactgcaaccgtactggctcaggctatcatcactgaaggtctgaaagctgttgctgcgggcatgaacccgatggacctgaaacgtggtatcgacaaagcggttaccgctgcagttgaagaactgaaagcgctgtccgtaccatgctctgactctaaagcgattgctcaggttggtaccatctccgctaactccgacgaaaccgtaggtaaactgatcgctgaagcgatggacaaagtcggtaaagaaggcgttatcaccgttgaagacggtaccggtctgcaggacgaactggacgtggttgaaggtatgcagttcgaccgtggctacctgtctccttacttcatcaacaagccggaaactggcgcagtagaactggaaagcccgttcatcctgctggctgacaagaaaatctccaacatccgcgaaatgctgccggttctggaagctgttgccaaagcaggcaaaccgctgctgatcatcgctgaagatgtagaaggcgaagcgctggcaactctggttgttaacaccatgcgtggcatcgtgaaagtcgctgcggttaaagcaccgggcttcggcgatcgtcgtaaagctatgctgcaggatatcgcaaccctgactggcggtaccgtgatctctgaagagatcggtatggagctggaaaaagcaaccctggaagacctgggtcaggctaaacgtgttgtgatcaacaaagacaccaccactatcatcgatggcgtgggtgaagaagctgcaatccagggccgtgttgctcagatccgtcagcagattgaagaagcaacttctgactacgaccgtgaaaaactgcaggaacgcgtagcgaaactggcaggcggcgttgcagttatcaaagtgggtgctgctaccgaagttgaaatgaaagagaaaaaagcacgcgttgaagatgccctgcacgcgacccgtgctgcggtagaagaaggcgtggttgctggtggtggtgttgcgctgatccgcgtagcgtctaaactggctgacctgcgtggtcagaacgaagaccagaacgtgggtatcaaagttgcactgcgtgcaatggaagctccgctgcgtcagatcgtattgaactgcggcgaagaaccgtctgttgttgctaacaccgttaaaggcggcgacggcaactacggttacaacgcagcaaccgaagaatacggcaacatgatcgacatgggtatcctggatccaaccaaagtaactcgttctgctctgcagtacgcagcttctgtggctggcctgatgatcaccaccgaatgcatggttaccgacctgccgaaaaacgatgcagctgacttaggcgctgctggcggtatgggcggcatgatgtaagtttaaacgcggccgcaatttgaacgccagcacatggactctcgagtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggtgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctggacaatgtctccatacccgtttttttgggcgacctcgtcggaggttgtatgtccggtgttccgtgacgtcatcgggcattcatcattcatagaatgtgttacggaggaaacaagtaatggcacttagcaccgcaaccaaggccgcgacggacgcgctggctgccaatcgggcacccaccagcgggaatgcacaggaagtgcaccgttggctccagagcttcaactgggatttcaagaacaaccggaccaagtacgccaccaagtacaagatggcgaacgagaccaaggaacagttcaagctgatcgccaaggaatatgcgcgcatggaggcagtcaaggacgaaaggcagttcggtagcctgcaggatgcgctgacccgcctcaacgccggtgttcgcgttcatccgaagtggaacgagaccatgaaagtggtttcgaacttcctggaagtgggcgaatacaacgccatcgccgctaccgggatgctgtgggattccgcccaggcggcggaacagaagaacggctatctggcccaggtgttggatgaaatccgccacacccaccagtgtgcctacgtcaactactacttcgcgaagaacggccaggacccggccggtcacaacgatgctcgccgcacccgtaccatcggtccgctgtggaagggcatgaagcgcgtgttttccgacggcttcatttccggcgacgccgtggaatgctccctcaacctgcagctggtgggtgaggcctgcttcaccaatccgctgatcgtcgcagtgaccgaatgggctgccgccaacggcgatgaaatcaccccgacggtgttcctgtcgatcgagaccgacgaactgcgccacatggccaacggttaccagaccgtcgtttccatcgccaacgatccggcttccgccaagtatctcaacacggacctgaacaacgccttctggacccagcagaagtacttcacgccggtgttgggcatgctgttcgagtatggctccaagttcaaggtcgagccgtgggtcaagacgtggaaccgctgggtgtacgaggactggggcggcatctggatcggccgtctgggcaagtacggggtggagtcgccgcgcagcctcaaggacgccaagcaggacgcttactgggctcaccacgacctgtatctgctggcttatgcgctgtggccgggcggcttcttccgtctggcgctgccggatcaggaagaaatggagtggttcgaggccaactaccccggctggtacgaccactacggcaagatctacgaggaatggcgcgcccgcggttgcgaggatccgtcctcgggcttcatcccgctgatgtggttcatcgaaaacaaccatcccatctacatcgatcgcgtgtcgcaagtgccgttctgcccgagcttggccaagggcgccagcaccctgcgcgtgcacgagtacaacggccagatgcacaccttcagcgaccagtggggcgagcgcatgtggctggccgagccggagcgctacgagtgccagaacatcttcgaacagtacgaaggacgcgaactgtcggaagtgatcgccgaactgcacgggctgcgcagtgatggcaagaccctgatcgcccagccgcatgtccgtggcgacaagctgtggacgttggacgatatcaaacgcctgaactgcgtcttcaagaacccggtgaaggcattcaattgaaacgggtgtcgggctccgtcacagggcggggcccgacgcacgatcgttcgatcaacctcaaaccaaaaaggaacatcgatatgagcatgttaggagaaagacgccgcggtctgaccgatccggaaatggcggccgtcattttgaaggcgcttcctgaagctccgctggacggcaacaacaagatgggttatttcgtcaccccccgctggaaacgcttgacggaatatgaagccctgaccgtttatgcgcagcccaacgccgactggatcgccggcggcctggactggggcgactggacccagaaattccacggcggccgcccttcctggggcaacgagaccacggagctgcgcaccgtcgactggttcaagcaccgtgacccgctccgccgttggcatgcgccgtacgtcaaggacaaggccgaggaatggcgctacaccgaccgcttcctgcagggttactccgccgacggtcagatccgggcgatgaacccgacctggcgggacgagttcatcaaccggtattggggcgccttcctgttcaacgaatacggattgttcaacgctcattcgcagggcgcccgggaggcgctgtcggacgtaacccgcgtcagcctggctttctggggcttcgacaagatcgacatcgcccagatgatccaactcgaacggggtttcctcgccaagatcgtacccggtttcgacgagtccacagcggtgccgaaggccgaatggacgaacggggaggtctacaagagcgcccgtctggccgtggaagggctgtggcaggaggtgttcgactggaacgagagcgctttctcggtgcacgccgtctatgacgcgctgttcggtcagttcgtccgccgcgagttctttcagcggctggctccccgcttcggcgacaatctgacgccattcttcatcaaccaggcccagacatacttccagatcgccaagcagggcgtacaggatctgtattacaactgtctgggtgacgatccggagttcagcgattacaaccgtaccgtgatgcgcaactggaccggcaagtggctggagcccacgatcgccgctctgcgcgacttcatggggctgtttgcgaagctgccggcgggcaccactgacaaggaagaaatcaccgcgtccctgtaccgggtggtcgacgactggatcgaggactacgccagcaggatcgacttcaaggcggaccgcgatcagatcgttaaagcggttctggcaggattgaaataatagaggaactattacgatgagcgtaaacagcaacgcatacgacgccggcatcatgggcctgaaaggcaaggacttcgccgatcagttctttgccgacgaaaaccaagtggtccatgaaagcgacacggtcgttctggtcctcaagaagtcggacgagatcaatacctttatcgaggagatccttctgacggactacaagaagaacgtcaatccgacggtaaacgtggaagaccgcgcgggttactggtggatcaaggccaacggcaagatcgaggtcgattgcgacgagatttccgagctgttggggcggcagttcaacgtctacgacttcctcgtcgacgtttcctccaccatcggccgggcctataccctgggcaacaagttcaccattaccagtgagctgatgggcctggaccgcaagctcgaagactctcacgcttaaggagaatgacatggcgaaactgggtatacacagcaacgacacccgcgacgcctgggtgaacaagatcgcgcagctcaacaccctggaaaaagcggccgagatgctgaagcagttccggatggaccacaccacgccgttccgcaacagctacgaactggacaacgactacctctggatcgaggccaagctcgaagagaaggtcgccgtcctcaaggcagaagccttcaacgaggtggacttccgtcataagaccgctttcggcgaggatgccaagtccgttctggacggcaccgtcgcgaagatgaacgcggccaaggacaagtgggaggcggagaagatccatatcggtttccgccaggcctacaagccgccgatcatgccggtgaactatttcctggacggcgagcgtcagttggggacccggctgatggaactgcgcaacctcaactactacgacacgccgctggaagaactgcgcaaacagcgcggtgtgcgggtggtgcatctgcagtcgccgcactgaagggaggaagtctcgccctggacgcgacggcatcgccgtgaagtccagggggcagggatgccgttccgggtcggcaggctggcccggaatctctggttttcagggggcgtgccggtccacggctcccccctccatctttcgtaaggaaatcaccatggtcgaatcggcatttcagccattttcgggcgacgcagacgaatggttcgaggaaccacggccccaggccggtttcttcccttccgcggactggcatctgctcaaacgggacgagacctacgcagcctatgccaaggatctcgatttcatgtggcggtgggtcatcgtccgggaagaaaggatcgtccaggagggttgctcgatcagcctggagtcgtcgatccgcgccgtgacgcacgtactgaattattttggtatgaccgaacaacgcgccccggcagaggaccggaccggcggagttcaacattgaacaggtaagtttatgcagcgagttcacactatcacggcggtgacggaggatggcgaatcgctccgcttcgaatgccgttcggacgaggacgtcatcaccgccgccctgcgccagaacatctttctgatgtcgtcctgccgggagggcggctgtgcgacctgcaaggccttgtgcagcgaaggggactacgacctcaagggctgcagcgttcaggcgctgccgccggaagaggaggaggaagggttggtgttgttgtgccggacctacccgaagaccgacctggaaatcgaactgccctatacccattgccgcatcagttttggtgaggtcggcagtttcgaggcggaggtcgtcggcctcaactgggtttcgagcaacaccgtccagtttcttttgcagaagcggcccgacgagtgcggcaaccgtggcgtgaaattcgaacccggtcagttcatggacctgaccatccccggcaccgatgtctcccgctcctactcgccggcgaaccttcctaatcccgaaggccgcctggagttcctgatccgcgtgttaccggagggacggttttcggactacctgcgcaatgacgcgcgtgtcggacaggtcctctcggtcaaagggccactgggcgtgttcggtctcaaggagcggggcatggcgccgcgctatttcgtggccggcggcaccgggttggcgccggtggtctcgatggtgcggcagatgcaggagtggaccgcgccgaacgagacccgcatctatttcggtgtgaacaccgagccggaattgttctacatcgacgagctcaaatccctggaacgatcgatgcgcaatctcaccgtgaaggcctgtgtctggcacccgagcggggactgggaaggcgagcagggctcgcccatcgatgcgttgcgggaagacctggagtcctccgacgccaacccggacatttatttgtgcggtccgccgggcatgatcgatgccgcctgcgagctggtacgcagccgcggtatccccggcgaacaggtcttcttcgaaaaattcctgccgtccggggcggcctgaaccggggaagtaccgtgaccaccgagcagttcccgccccaattcctgcgtgaaatgatcgagcagctggacgccagcatccaggagctcgcacgcaaggaaaagggacttgcggcatccctgggcacgggccgggtcgccgagctcaaggaatactgggaccacgttgttacaaccaattaaccaattctgactatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcg

SEQ ID NO: 45 - DNA, 6364 bp, Artificial Sequence
  Description of Artificial Sequence: Synthetic polynucleotide
cggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggctggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctcccaatgtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttttgacggctagctcagtcctagggataatgctagcaccagcctcgagggaaaccacgtaagctccggcgtttaaacacccataacagatacggactttctcaaaggagagttatcagtgaaaatccgcccgttacatgaccgtgtcatcatcaaacgcttggaagaagagcgtacctcggcgggcgggattgtcattccagatagcgcagctgaaaaaccgatgcgtggtgaaatcctggcagtgggcaatggaaaagtgcttgataatggagaggtacgtgctttacaggtgaaagtgggtgataaagtgctctttgggaaatacgcgggtacggaggttaaagtagatggggaagatgttgttgtcatgcgtgaagatgacattctggctgtgttagaatcttaatccgcgcacgacactgaacatacgaatttaaggaataaagataatggcgaaagaagttgtgtatcgtggtagtgcgcgccagcgtatgatgcagggtattgaaattctcgctcgcgccgctattccaacgctgggggcaaccggcccgagcgtcatgattcaacatcgcgccgatggtctgccacccatttctacacgcgatggcgttaccgtagcgaattctattgttttaaaagaccgtgtcgcgaacctgggtgcccgcctgctgcgcgacgtagccggtacaatgagccgtgaagccggcgacggcacgacgactgcgatcgtattggcccgccacatcgcccgtgagatgtttaaatcgctggccgtgggtgcagatccgatcgcgctgaaacgtggtatcgatcgcgccgttgctcgtgtgtccgaagatattggggcgcgtgcgtggcgtggcgataaagaaagcgtgatcctgggtgtcgctgctgtggcgacgaaaggcgaaccgggcgttggccgtctgctgctggaggctctcgatgcagtgggtgttcacggtgccgtttctatcgaactgggccaacgtcgtgaagatctgctggacgtcgtcgatggctatcgctgggaaaaaggttatttatctccctactttgtcacggaccgtgcccgcgaactcgcggaactggaggatgtctacctgctcatgaccgaccgcgaagtggttgacttcatcgaccttgtacctctgctggaggccgtgacggaagcaggaggctccctgctgattgccgcggatcgtgtgcacgaaaaggccttagcggggctgcttctgaatcacgtgcgcggtgtcttcaaggccgtggccgtaaccgctccgggttttggcgacaaacgcccgaaccgtttacttgacctggccgcgttaaccggcggtcgtgccgtgctcgaagctcaaggcgaccgtctggaccgtgttaccctcgcggatctgggccgtgtgcgccgtgccgtggtgtcggcagatgataccgcgctgcttggcatcccgggcaccgaagctagccgtgcacgcctcgaaggtctgcgtttagaagcagagcagtaccgtgcgctgaaaccagggcagggttctgccaccgggcgcctgcacgaacttgaagaaattgaagcgcgcattgtgggtctgtccggaaagagcgccgtttatcgcgtcggaggtgtgaccgatgtggaaatgaaagagcgcatggttcgcatcgaaggagcttaccgttcggtggtaagtgcgctggaggaaggcgtgctccctggcggtggtgtcggctttctgggtagtatgccggtgcttgcggaattggaggcccgcgacgcagatgaagctcgcgggattgggattgtacgcagcgccttaacggagcctcttcgtattatcggcgaaaatagtggcttgagcggtgaagccgttgttgccaaagtcatggatcatgccaacccgggatggggttacgaccaggagtctggctctttttgcgacctgcatgcgcgtgggatctgggatgctgctaaagtgttacgtctcgcgttggagaaggcagcctctgttgctgggacctttctgacaaccgaagctgttgttctcgaaattccggatacagatgcgttcgcagggttcagtgcagaatgggctgccgccacgcgcgaagatccgcgcgtatgagtttaaacgcggccgcaatttgaacgcacccataacagatacggactttctcaaaggagagttatcaatgaatattcgtccattgcatgatcgcgtgatcgtcaagcgtaaagaagttgaaactaaatctgctggcggcatcgttctgaccggctctgcagcggctaaatccacccgcggcgaagtgctggctgtcggcaatggccgtatccttgaaaatggcgaagtgaagccgctggatgtgaaagttggcgacatcgttattttcaacgatggctacggtgtgaaatctgagaagatcgacaatgaagaagtgttgatcatgtccgaaagcgacattctggcaattgttgaagcgtaatccgcgcacgacactgaacatacgaatttaaggaataaagataatggcagctaaagacgtaaaattcggtaacgacgctcgtgtgaaaatgctgcgcggcgtaaacgtactggcagatgcagtgaaagttaccctcggtccaaaaggccgtaacgtagttctggataaatctttcggtgcaccgaccatcaccaaagatggtgtttccgttgctcgtgaaatcgaactggaagacaagttcgaaaatatgggtgcgcagatggtgaaagaagttgcctctaaagcaaacgacgctgcaggcgacggtaccaccactgcaaccgtactggctcaggctatcatcactgaaggtctgaaagctgttgctgcgggcatgaacccgatggacctgaaacgtggtatcgacaaagcggttaccgctgcagttgaagaactgaaagcgctgtccgtaccatgctctgactctaaagcgattgctcaggttggtaccatctccgctaactccgacgaaaccgtaggtaaactgatcgctgaagcgatggacaaagtcggtaaagaaggcgttatcaccgttgaagacggtaccggtctgcaggacgaactggacgtggttgaaggtatgcagttcgaccgtggctacctgtctccttacttcatcaacaagccggaaactggcgcagtagaactggaaagcccgttcatcctgctggctgacaagaaaatctccaacatccgcgaaatgctgccggttctggaagctgttgccaaagcaggcaaaccgctgctgatcatcgctgaagatgtagaaggcgaagcgctggcaactctggttgttaacaccatgcgtggcatcgtgaaagtcgctgcggttaaagcaccgggcttcggcgatcgtcgtaaagctatgctgcaggatatcgcaaccctgactggcggtaccgtgatctctgaagagatcggtatggagctggaaaaagcaaccctggaagacctgggtcaggctaaacgtgttgtgatcaacaaagacaccaccactatcatcgatggcgtgggtgaagaagctgcaatccagggccgtgttgctcagatccgtcagcagattgaagaagcaacttctgactacgaccgtgaaaaactgcaggaacgcgtagcgaaactggcaggcggcgttgcagttatcaaagtgggtgctgctaccgaagttgaaatgaaagagaaaaaagcacgcgttgaagatgccctgcacgcgacccgtgctgcggtagaagaaggcgtggttgctggtggtggtgttgcgctgatccgcgtagcgtctaaactggctgacctgcgtggtcagaacgaagaccagaacgtgggtatcaaagttgcactgcgtgcaatggaagctccgctgcgtcagatcgtattgaactgcggcgaagaaccgtctgttgttgctaacaccgttaaaggcggcgacggcaactacggttacaacgcagcaaccgaagaatacggcaacatgatcgacatgggtatcctggatccaaccaaagtaactcgttctgctctgcagtacgcagcttctgtggctggcctgatgatcaccaccgaatgcatggttaccgacctgccgaaaaacgatgcagctgacttaggcgctgctggcggtatgggcggcatgatgtaagtttaaacgcggccgcaatttgaacgccagcacatggactctcgagtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccagctcaaggaatactgggaccacgttgttacaaccaattaaccaattctgactatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactg

SEQ ID NO: 46 - DNA, 7032 bp, Artificial Sequence
  Description of Artificial Sequence: Synthetic polynucleotide
ccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatttcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcacaattctcatgtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagccagtccgtttaggtgttttcacgagcaattgaccaacaaggacgtcaatagagttaaggagggaggggatgaccactgctgcaccccaagaatttaccgctgctgttgttgaaaaattcggtcatgacgtgaccgtgaaggatattgaccttccaaagccagggccacaccaggcattggtgaaggtactcacctccggcatctgccacaccgacctccacgccttggagggcgattggccagtaaagccggaaccaccattcgtaccaggacacgaaggtgtaggtgaagttgttgagctcggaccaggtgaacacgatgtgaaggtcggcgatattgtcggcaatgcgtggctctggtcagcgtgtggcacctgcgaatactgcatcaccggcagggaaactcagtgcaacgaagctgagtatggtggctacacccaaaatggatccttcggccagtacatgctggtggatacccgttacgccgctcgcatcccagacggcgtggactacctcgaagcagcaccaattctgtgtgcaggcgtgactgtctacaaggcactcaaagtctctgaaacccgcccgggccaattcatggtgatctccggtgtcggcggacttggccacatcgcagtccaatacgcagcggcgatgggcatgcgtgtcattgcggtagatattgccgatgacaagctggaacttgcccgtaagcacggtgcggaatttaccgtgaatgcgcgtaatgaagattcaggcgaagctgtacagaagtacaccaacggtggcgcacacggcgtgcttgtgactgcagttcacgaggcagcattcggccaggcactggatatggctcgacgtgcaggaacaattgtgttcaacggtctgccaccgggagagttcccagcatccgtgttcaacatcgtattcaagggcctgaccatccgtggatccctcgtgggaacccgccaagacttggccgaagcgctcgatttctttgcacgcggactaatcaagccaaccgtgagtgagtgctccctcgatgaggtcaatggtgtgcttgaccgcatgcgaaacggcaagatcgatggtcgtgtggcgattcgtttctaacatgctaaggtgctggctgcatgctaagttgatacgcctgcgacaaatttttctaggagcgttagtatgactgtctacgcaaatccaggaaccgaaggctcgatcgttaactatgaaaagcgctacgagaactacattggtggcaagtgggttccaccggtagagggccagtaccttgagaacatttcacctgtcactggtgaagttttctgtgaggtcgcacgtggcaccgcagcggacgtggagcttgcactggatgctgcacatgcagccgctgatgcgtggggcaagacttctgtcgctgaacgtgctctgatcctgcaccgcattgcggaccgcatggaagagcacctggaagaaatcgcagttgcagaaacctgggagaacggcaaggcagtccgtgagactcttgctgcagatatcccactggcaatcgaccacttccgctactttgctggcgcgatccgtgctcaggaagatcgttcctcacagatcgaccacaacactgttgcttaccacttcaacgagccaatcggtgttgttggtcagatcattccttggaacttcccaatcctcatggctacctggaagctcgcaccggcacttgctgcaggtaacgcgatcgtcatgaagccagctgagcagaccccagcatccattttgtatctgattaacatcatcggcgatctcatcccagagggcgtcctcaacatcgtcaacggactcggcggtgaagcaggcgctgcactgtccggctctaatcggattggcaagattgctttcaccggttccaccgaggtcggcaagctgatcaaccgcgctgcatccgacaagatcattcctgtcaccctggagctcggcggtaagtccccatccatcttcttctccgatgttctgtcacaggatgacgccttcgcagagaaggcagttgaaggcttcgcgatgttcgccctcaatcagggtgaagtttgtacctgtccttcccgtgcacttgttcatgagtccatcgctgatgaattcctcgagcttggcgtgaagcgagttcagaacatcaagctgggtaacccacttgatactgaaaccatgatgggtgctcaggcgtcccaggagcagatggacaagatctcctcctacctgaagatcggcccagaagaaggcgctcaaaccctcactggtggcaaggtcaacaaggttgatggcatggagaacggttactacattgagccaaccgttttccgcggcaccaacgacatgaggatcttccgcgaggaaatcttcggaccagtcctttctgttgctaccttcagcgacttcgatgaggccatccgtattgcaaacgacaccaactacggcctcggcgctggtgtctggagccgtgaccaaaacaccatttatcgtgcaggtcgcgcaatccaggctggtcgagtttgggtcaaccagtaccacaactacccagcgcactccgctttcggtggatacaaggagtccggcatcggccgtgagaaccacctcatgatgctgaaccactaccagcagaccaagaacctgttggtctcctacgatccaaacccaaccggactgttctgagtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttcggtccgttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgtttcccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttccttaggactgagcgtcaaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatataaggtgcactgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgcagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgaaatt

SEQ ID NO: 47 - DNA, 7162 bp, Artificial Sequence
  Description of Artificial Sequence: Synthetic polynucleotide
gccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaataaaaaatgccctcttgggttatcaagagggtcattatatttcgcaaaaaaccccgcttcggcggggttttttcgcttgacggctagctcagtcctaggtacagtgctagcaattaaaggaggcctaatgaccactgctgcaccccaagaatttactgctgctgttgttgaaaaattcggtcatgacgtgaccgtgaaggatattgaccttccaaagccagggccacaccaggcattggtgaaggtactcacctccggcatctgccacaccgacctccacgccttggagggcgattggccagtaaagccggaaccaccattcgtaccaggacacgaaggtgtaggtgaagttgttgagctcggaccaggtgaacacgatgtgaaggtcggcgatattgtcggcaatgcgtggctctggtcagcgtgtggcacctgcgaatactgcatcaccggcagggaaactcagtgcaacgaagctgagtatggtggctacacccaaaatggatccttcggccagtacatgctggtggatacccgttacgccgctcgcatcccagacggcgtggactacctcgaagcagcaccaattctgtgtgcaggcgtgactgtctacaaggcactcaaagtctctgaaacccgcccgggccaattcatggtgatctccggtgtcggcggacttggccacatcgcagtccaatacgcagcggcgatgggcatgcgtgtcattgcggtagatattgccgatgacaagctggaacttgcccgtaagcacggtgcggaatttaccgtgaatgcgcgtaatgaagattcaggcgaagctgtacagaagtacaccaacggtggcgcacacggcgtgcttgtgactgcagttcacgaggcagcattcggccaggcactggatatggctcgacgtgcaggaacaattgtgttcaacggtctgccaccgggagagttcccagcatccgtgttcaacatcgtattcaagggcctgaccatccgtggatccctcgtgggaacccgccaagacttggccgaagcgctcgatttctttgcacgcggactaatcaagccaaccgtgagtgagtgctccctcgatgaggtcaatggtgtgcttgaccgcatgcgaaacggcaagattgatggtcgtgtggcaattcgctactaagcatggacgagctgtacaagtaagactgctaaagcgtcaaaaggccggattttccggccttttttattactaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgctcctaagtcatttagatattggctgggatgttcagggtgatttcgttaatatggcgttgggagtgtaaaagttgagcggtccatgcgcaggcgaactccaaaccctccagcgagcggtttgcaaaacgtacgatttggtttgcccatacgggcggcagaacatggtcgcccgcggcagacgcgcgttgatagtccagctcggcttcgtggcgccaaacacgaattaattgaccgatggctgccgactgaatgcggccataaacatttccgttctgatctgccccattagacagaaagatcacacgtggtccgcgggcgcgcgccccaggcgtcaggcgttgtgactgccagtagcgagcaagacggctagcaatgacaatcgtcccggtgatttcgtccgcaagaaagttcagcacacgttcgtcatctacttcaattacacacggtgcgggctcatgggatgtcgcgggcaggataaccgccgcatgaatacccccagtgttttctccagctccccaatcgaatacagcgtcgatcgtagcagggtcacgcggatcaagaggtaaggcgatcggcggggtaaagtctgcgccagctaaacgacgcgactcatttactgcctgttcaaactgtgctaatgcggcggcgctacgaaaaccaataattacctccgatccgcatgtgcgcagcatacccgttaaagccattacctcctcaatctgatccccggcacagataagggtggtacggccagaggcatcgatagtgcgcagatcagtgcgcgctaacagactcgtttccgaacaggccggcagctccattccatgggtcacctcaatcgtttccccagataatgctgcagactcagcagaagccaagaagaccgcggcatcggcgacatcgccgacggaagggaagcggcgttctaatgccccttggtcgtttgcgcgacacaggcgcatcgtgttcaagaaatggtgcgccgtgtcaccctcaggacggcccttcagctgatccatacgttgaaatacagtgcgaatgcggtccgattcgatgggacccggaaagattgtatttacgcgaattccacgcgcgcccagttcgcgcgcagccagctggcttaacgcattcagtgcggctttgggggtcacatatgggatacgtccgtagtattcggcacgagaaaagatggtggacacattgataacagctgatccaacgggcatgtgcggggccgcaatgcgcatcagatgccagcccattccaagaagattggcgatactagcgtgcaatgtttcctctgctccgggacccaattctgcttcagttaaagggatttccgctaaacgacgctgagcaccagcgctaccggcgttgttgacaagaatgtcaatctggccgtggcgagcaacaatagcctcaatgcctgcgcgcacggcaactgggtcgcttccgtccataacttccaagtcaatgcgtttagcggggacgccggcctcagcttgcatacgctcggccagcgcagtcagcttagcacgattacgtccagagataatgactgtcgcaccctctgccaggaagcgacgggttaattcggagccaatattgccggcacctcccgtaatcaaggcaatttttcccgccaagcgccctgttcctgacattgagtacctcctttcttaattataccgtaatcgcgcgaccgcggtggatacgtccggcataacgagtgtccgcttcgggtggcaacggtgcggtaactaacagcacggcttcgatgaagcgctccaattcctgttgacgttcgtgagggtcacgcggctcctctgcgcgagcacgacgagttaaatcgacttgattgatcaggatgcgctgcgctgtgcgttcagactctacaccgatcgttgcggtgaaagcatgaagcgttgtcttaatgaagttggccaaggcgaactgctcggttgtgctagttgccgtagtctcgggcgtgactaaggcaagacgtgcaccgtcagacagggcgatccaacgtgctacacggaagtgatgcgttaactgatgctcacataactcagcgaattctgcctcgctcaagacggtagaccaatcgctgtccttgcggcctacaagaggaacagtgggcaatggacgaaaaggggtacagactacgggacctggacgtccgtaacgagtaatcgcttggtcaatcgcggcctcgatctggtcaccagccacaatcgtcatcaagcgtcctgcctctacgtggtcgtgcagtaaacgacgcatggtttcagctccagtttccgtttctacgatcataactacctggcgtgccccgtagcgctctaagtaagcgcgtgcaagcaagtttaaatgctcagttaaatgttcaccgatcagatagactgtggagccgacaagctctgctaaacgttccggcgaaggcaggccaaacaattctccccctgtcggggtgcgttcgtagcgcaaacctccagacggatggaaagtctcgcctgagacaacacggtccgccaaataataaaccgtcgccatcgcaacatcaaattccgtaggcatgcgctgaagataaagcattcccataattccatcgcgtaccttacgagcctcgcggtcgatctgggcgcgggtgaaaaaagggtctggtgggttcggcaaattggcaaaaatgtctgctggcagaacatatccaccattgtgcaaacgagcaagtaatttcgccgcaatggagcgatttaataaagcactggacgaagacgctgcagggtctccctcactgcggaagcgacgcgccagctcacgaagtgcggtaggggcagcggggttttgttcaagcgccgcgacgtcattcggcaacaacaattcaaccaactcatgcatcgaacgttcatcggtacgagctgccgcgattaacgcagcatgaagctcatttaaacgcttgttctccaagataaggcgtgcacggcgagcgaacaatccaggacgctcaccagttccgcgcagacgatctccctctacgggtccaggtgcaatagcattgatctgaatctcgggtcccaaaaagcgcgcaaagacttcagccatagcgcgctggccggctttactaacagcatagtcggcacggttgggatacggaattgctgcgtctttctcccctccgaaatacgagcttacgttgaggatgtaccccgacccctgcttcttcatcagtggtgccagcttacgcataaggctgtagttcgagatgaggtttgcgaacagcgtatgacgccaaccctccacgggcatgtcgataaccatctcctctacacccgcaatgccggcgttattgattaaatagtcaacggtaccgaaagctgacaatgtgcgttcaaccaagtctgcaagttgggcctcacttgaaacgtcgcaccctggtgcgatatgaacgcgatcctccacatctgtgtagccaacttccgctaattcactctgaatcatagcttgcatctgttccaatttgtggcgatcacgtgccgccagcatcacacgtgcaccagataaagccaataagcgaccaatctgcccaccaatgccggcagatccacccgtaatcagggcgaccttgcccaaatgcaacccaatcagtgattctgcccatccaacagaggcgctacgtgcgcccgtcgtcgccgacattaatacctcctgtccttgttggtcaattgctcgtgaaaacacctaaacggactggctgtttcctgtgtgagctagcattatacctaggactgagctagctgtcaattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatga

SEQ ID NO: 48 - DNA, 8111 bp, Escherichia coli
gaacccccaggatgcgcatagcgcttatattcgcggtgacgtggagttggtgcggattcgtgatgccgaagggcgaattgcggcagaaggggcgttgccttatccacctggcgtgctttgcgtggtacccggggaagtctggggtggggcggttcaacgttatttccttgcactggaagaaggggtgaatttgttgccgggattttcgccggagctgcaaggtgtttatagcgaaaccgatgcggatggcgtgaaacggttgtacggttatgtgttgaagtaagaataaaaaaaacgggtcaccttctggcgacccgtttttctttgcgtaattagtggctaaccgtctgtgtgcctgtcgggacacgaacgtgtttatatttgaacatcgccatgaacgcgaaggccagaaccacggagtaaccagcgaaaatcaaccatacggtctgccagtcggtaatgccgttttgggtgtacatctcaacaactttaccgctcacgatgccgccgaggatacagccgaagccgttagtcatcatcaggaacatcccttgtgcactggcgcgaattgccgggctaacttctttttcgacaaacaccgaaccagagatgttgaagaagtcgaatgcgcaaccgtaaacgatcatcgacagtaccagcagtacagtacctcacactgcttccggtagtcaataaaccggtaaaccagcaatagacataagcggtgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctggacaatgtctccatacccgtttttttgggcgacctcgtcggaggttgtatgtccggtgttccgtgacgtcatcgggcattcatcattcatagaatgtgttacggaggaaacaagtaatggcacttagcaccgcaaccaaggccgcgacggacgcgctggctgccaatcgggcacccaccagcgggaatgcacaggaagtgcaccgttggctccagagcttcaactgggatttcaagaacaaccggaccaagtacgccaccaagtacaagatggcgaacgagaccaaggaacagttcaagctgatcgccaaggaatatgcgcgcatggaggcagtcaaggacgaaaggcagttcggtagcctgcaggatgcgctgacccgcctcaacgccggtgttcgcgttcatccgaagtggaacgagaccatgaaagtggtttcgaacttcctggaagtgggcgaatacaacgccatcgccgctaccgggatgctgtgggattccgcccaggcggcggaacagaagaacggctatctggcccaggtgttggatgaaatccgccacacccaccagtgtgcctacgtcaactactacttcgcgaagaacggccaggacccggccggtcacaacgatgctcgccgcacccgtaccatcggtccgctgtggaagggcatgaagcgcgtgttttccgacggcttcatttccggcgacgccgtggaatgctccctcaacctgcagctggtgggtgaggcctgcttcaccaatccgctgatcgtcgcagtgaccgaatgggctgccgccaacggcgatgaaatcaccccgacggtgttcctgtcgatcgagaccgacgaactgcgccacatggccaacggttaccagaccgtcgtttccatcgccaacgatccggcttccgccaagtatctcaacacggacctgaacaacgccttctggacccagcagaagtacttcacgccggtgttgggcatgctgttcgagtatggctccaagttcaaggtcgagccgtgggtcaagacgtggaaccgctgggtgtacgaggactggggcggcatctggatcggccgtctgggcaagtacggggtggagtcgccgcgcagcctcaaggacgccaagcaggacgcttactgggctcaccacgacctgtatctgctggcttatgcgctgtggccgggcggcttcttccgtctggcgctgccggatcaggaagaaatggagtggttcgaggccaactaccccggctggtacgaccactacggcaagatctacgaggaatggcgcgcccgcggttgcgaggatccgtcctcgggcttcatcccgctgatgtggttcatcgaaaacaaccatcccatctacatcgatcgcgtgtcgcaagtgccgttctgcccgagcttggccaagggcgccagcaccctgcgcgtgcacgagtacaacggccagatgcacaccttcagcgaccagtggggcgagcgcatgtggctggccgagccggagcgctacgagtgccagaacatcttcgaacagtacgaaggacgcgaactgtcggaagtgatcgccgaactgcacgggctgcgcagtgatggcaagaccctgatcgcccagccgcatgtccgtggcgacaagctgtggacgttggacgatatcaaacgcctgaactgcgtcttcaagaacccggtgaaggcattcaattgaaacgggtgtcgggctccgtcacagggcggggcccgacgcacgatcgttcgatcaacctcaaaccaaaaaggaacatcgatatgagcatgttaggagaaagacgccgcggtctgaccgatccggaaatggcggccgtcattttgaaggcgcttcctgaagctccgctggacggcaacaacaagatgggttatttcgtcaccccccgctggaaacgcttgacggaatatgaagccctgaccgtttatgcgcagcccaacgccgactggatcgccggcggcctggactggggcgactggacccagaaattccacggcggccgcccttcctggggcaacgagaccacggagctgcgcaccgtcgactggttcaagcaccgtgacccgctccgccgttggcatgcgccgtacgtcaaggacaaggccgaggaatggcgctacaccgaccgcttcctgcagggttactccgccgacggtcagatccgggcgatgaacccgacctggcgggacgagttcatcaaccggtattggggcgccttcctgttcaacgaatacggattgttcaacgctcattcgcagggcgcccgggaggcgctgtcggacgtaacccgcgtcagcctggctttctggggcttcgacaagatcgacatcgcccagatgatccaactcgaacggggtttcctcgccaagatcgtacccggtttcgacgagtccacagcggtgccgaaggccgaatggacgaacggggaggtctacaagagcgcccgtctggccgtggaagggctgtggcaggaggtgttcgactggaacgagagcgctttctcggtgcacgccgtctatgacgcgctgttcggtcagttcgtccgccgcgagttctttcagcggctggctccccgcttcggcgacaatctgacgccattcttcatcaaccaggcccagacatacttccagatcgccaagcagggcgtacaggatctgtattacaactgtctgggtgacgatccggagttcagcgattacaaccgtaccgtgatgcgcaactggaccggcaagtggctggagcccacgatcgccgctctgcgcgacttcatggggctgtttgcgaagctgccggcgggcaccactgacaaggaagaaatcaccgcgtccctgtaccgggtggtcgacgactggatcgaggactacgccagcaggatcgacttcaaggcggaccgcgatcagatcgttaaagcggttctggcaggattgaaataatagaggaactattacgatgagcgtaaacagcaacgcatacgacgccggcatcatgggcctgaaaggcaaggacttcgccgatcagttctttgccgacgaaaaccaagtggtccatgaaagcgacacggtcgttctggtcctcaagaagtcggacgagatcaatacctttatcgaggagatccttctgacggactacaagaagaacgtcaatccgacggtaaacgtggaagaccgcgcgggttactggtggatcaaggccaacggcaagatcgaggtcgattgcgacgagatttccgagctgttggggcggcagttcaacgtctacgacttcctcgtcgacgtttcctccaccatcggccgggcctataccctgggcaacaagttcaccattaccagtgagctgatgggcctggaccgcaagctcgaagactctcacgcttaaggagaatgacatggcgaaactgggtatacacagcaacgacacccgcgacgcctgggtgaacaagatcgcgcagctcaacaccctggaaaaagcggccgagatgctgaagcagttccggatggaccacaccacgccgttccgcaacagctacgaactggacaacgactacctctggatcgaggccaagctcgaagagaaggtcgccgtcctcaaggcagaagccttcaacgaggtggacttccgtcataagaccgctttcggcgaggatgccaagtccgttctggacggcaccgtcgcgaagatgaacgcggccaaggacaagtgggaggcggagaagatccatatcggtttccgccaggcctacaagccgccgatcatgccggtgaactatttcctggacggcgagcgtcagttggggacccggctgatggaactgcgcaacctcaactactacgacacgccgctggaagaactgcgcaaacagcgcggtgtgcgggtggtgcatctgcagtcgccgcactgaagggaggaagtctcgccctggacgcgacggcatcgccgtgaagtccagggggcagggatgccgttccgggccggcaggctggcccggaatctctggttttcagggggcgtgccggtccacggctcccccctccatctttcgtaaggaaatcaccatggtcgaatcggcatttcagccattttcgggcgacgcagacgaatggttcgaggaaccacggccccaggccggtttcttcccttccgcggactggcatctgctcaaacgggacgagacctacgcagcctatgccaaggatctcgatttcatgtggcggtgggtcatcgtccgggaagaaaggatcgtccaggagggttgctcgatcagcctggagtcgtcgatccgcgccgtgacgcacgtactgaattattttggtatgaccgaacaacgcgccccggcagaggaccggaccggcggagttcaacattgaacaggtaagtttatgcagcgagttcacactatcacggcggtgacggaggatggcgaatcgctccgcttcgaatgccgttcggacgaggacgtcatcaccgccgccctgcgccagaacatctttctgatgtcgtcctgccgggagggcggctgtgcgacctgcaaggccttgtgcagcgaaggggactacgacctcaagggctgcagcgttcaggcgctgccgccggaagaggaggaggaagggttggtgttgttgtgccggacctacccgaagaccgacctggaaatcgaactgccctatacccattgccgcatcagttttggtgaggtcggcagtttcgaggcggaggtcgtcggcctcaactgggtttcgagcaacaccgtccagtttcttttgcagaagcggcccgacgagtgcggcaaccgtggcgtgaaattcgaacccggtcagttcatggacctgaccatccccggcaccgatgtctcccgctcctactcgccggcgaaccttcctaatcccgaaggccgcctggagttcctgatccgcgtgttaccggagggacggttttcggactacctgcgcaatgacgcgcgtgtcggacaggtcctctcggtcaaagggccactgggcgtgttcggtctcaaggagcggggcatggcgccgcgctatttcgtggccggcggcaccgggttggcgccggtggtctcgatggtgcggcagatgcaggagtggaccgcgccgaacgagacccgcatctatttcggtgtgaacaccgagccggaattgttctacatcgacgagctcaaatccctggaacgatcgatgcgcaatctcaccgtgaaggcctgtgtctggcacccgagcggggactgggaaggcgagcagggctcgcccatcgatgcgttgcgggaagacctggagtcctccgacgccaacccggacatttatttgtgcggtccgccgggcatgatcgatgccgcctgcgagctggtacgcagccgcggtatccccggcgaacaggtcttcttcgaaaaattcctgccgtccggggcggcctgaaccggggaagtaccgtgaccaccgagcagttcccgccccaattcctgcgtgaaatgatcgagcagctggacgccagcatccaggagctcgcacgcaaggaaaagggacttgcggcatccctgggcacgggccgggtcgccgagctcaaggaatactgggaccacgtagtcgggtcgccgtaagcaaacagcgcaaaacgcaggatccacgccacaatactgatcatcattacgttcttaataccgtagcggcttaagaagaacgggatggtcagaatgaacagggtttcagagatctgcgaaatcgacatgatgattgacgcatgctgcacaataaagctgctggcaaacatcggatctttgtcgaagctgtgcaggaaggtattaccgaacatgttggtaatctgcagttccgcgcccagcagcattgagaagataaagaagattgccatacgcttgtttttaaacagcgcgaatgcatcgaggcccagcagggttgtccagctctgattcgcttgctgtttagcaaccggaatatgcggcagagtcagggtaaacagaaccagaatggcggaaagtgctgcgccaatatacagctgcatgtggcttaattcgaagccagacaggctcaccacccacattgccatgataaagccgatggtgccccagatacggattggc

SEQ ID NO: 49 - DNA, 7757 bp, Escherichia coli
ttttctcaagttctgccagcttctgctgggtttctttgtcttgtgcaggatgctgcatcaccggcatcatgttgtcggcaatagtggtcgccggtttaaccagatactggtttgggtgatcgtatccggcaaagcctttgcgtgcggtggcggttgacggggtatctgctgcgttggccatgagtggaagagcggcggcgacagctacaacaagaagtttgggtgaaaacgaaaattccatgcaaaatgctccggtttcatgtcgtcaaaatgttgacgtaattaagcattgataattgataattgagatccctctccctgacaggatgattgcataaataatagtgatgaaaataaattatttatttatccagaaaatgaattggaaaatcaggagagcgttttcaatcctacccctaactgactatggccttctgtagtcacactgcttccggtagtcaataaaccggtaaaccagcaatagacataagcggtgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctggacaatgtctccatacccgtttttttgggcgacctcgtcggaggttgtatgtccggtgttccgtgacgtcatcgggcattcatcattcatagaatgtgttacggaggaaacaagtaatggcacttagcaccgcaaccaaggccgcgacggacgcgctggctgccaatcgggcacccaccagcgggaatgcacaggaagtgcaccgttggctccagagcttcaactgggatttcaagaacaaccggaccaagtacgccaccaagtacaagatggcgaacgagaccaaggaacagttcaagctgatcgccaaggaatatgcgcgcatggaggcagtcaaggacgaaaggcagttcggtagcctgcaggatgcgctgacccgcctcaacgccggtgttcgcgttcatccgaagtggaacgagaccatgaaagtggtttcgaacttcctggaagtgggcgaatacaacgccatcgccgctaccgggatgctgtgggattccgcccaggcggcggaacagaagaacggctatctggcccaggtgttggatgaaatccgccacacccaccagtgtgcctacgtcaactactacttcgcgaagaacggccaggacccggccggtcacaacgatgctcgccgcacccgtaccatcggtccgctgtggaagggcatgaagcgcgtgttttccgacggcttcatttccggcgacgccgtggaatgctccctcaacctgcagctggtgggtgaggcctgcttcaccaatccgctgatcgtcgcagtgaccgaatgggctgccgccaacggcgatgaaatcaccccgacggtgttcctgtcgatcgagaccgacgaactgcgccacatggccaacggttaccagaccgtcgtttccatcgccaacgatccggcttccgccaagtatctcaacacggacctgaacaacgccttctggacccagcagaagtacttcacgccggtgttgggcatgctgttcgagtatggctccaagttcaaggtcgagccgtgggtcaagacgtggaaccgctgggtgtacgaggactggggcggcatctggatcggccgtctgggcaagtacggggtggagtcgccgcgcagcctcaaggacgccaagcaggacgcttactgggctcaccacgacctgtatctgctggcttatgcgctgtggccgggcggcttcttccgtctggcgctgccggatcaggaagaaatggagtggttcgaggccaactaccccggctggtacgaccactacggcaagatctacgaggaatggcgcgcccgcggttgcgaggatccgtcctcgggcttcatcccgctgatgtggttcatcgaaaacaaccatcccatctacatcgatcgcgtgtcgcaagtgccgttctgcccgagcttggccaagggcgccagcaccctgcgcgtgcacgagtacaacggccagatgcacaccttcagcgaccagtggggcgagcgcatgtggctggccgagccggagcgctacgagtgccagaacatcttcgaacagtacgaaggacgcgaactgtcggaagtgatcgccgaactgcacgggctgcgcagtgatggcaagaccctgatcgcccagccgcatgtccgtggcgacaagctgtggacgttggacgatatcaaacgcctgaactgcgtcttcaagaacccggtgaaggcattcaattgaaacgggtgtcgggctccgtcacagggcggggcccgacgcacgatcgttcgatcaacctcaaaccaaaaaggaacatcgatatgagcatgttaggagaaagacgccgcggtctgaccgatccggaaatggcggccgtcattttgaaggcgcttcctgaagctccgctggacggcaacaacaagatgggttatttcgtcaccccccgctggaaacgcttgacggaatatgaagccctgaccgtttatgcgcagcccaacgccgactggatcgccggcggcctggactggggcgactggacccagaaattccacggcggccgcccttcctggggcaacgagaccacggagctgcgcaccgtcgactggttcaagcaccgtgacccgctccgccgttggcatgcgccgtacgtcaaggacaaggccgaggaatggcgctacaccgaccgcttcctgcagggttactccgccgacggtcagatccgggcgatgaacccgacctggcgggacgagttcatcaaccggtattggggcgccttcctgttcaacgaatacggattgttcaacgctcattcgcagggcgcccgggaggcgctgtcggacgtaacccgcgtcagcctggctttctggggcttcgacaagatcgacatcgcccagatgatccaactcgaacggggtttcctcgccaagatcgtacccggtttcgacgagtccacagcggtgccgaaggccgaatggacgaacggggaggtctacaagagcgcccgtctggccgtggaagggctgtggcaggaggtgttcgactggaacgagagcgctttctcggtgcacgccgtctatgacgcgctgttcggtcagttcgtccgccgcgagttctttcagcggctggctccccgcttcggcgacaatctgacgccattcttcatcaaccaggcccagacatacttccagatcgccaagcagggcgtacaggatctgtattacaactgtctgggtgacgatccggagttcagcgattacaaccgtaccgtgatgcgcaactggaccggcaagtggctggagcccacgatcgccgctctgcgcgacttcatggggctgtttgcgaagctgccggcgggcaccactgacaaggaagaaatcaccgcgtccctgtaccgggtggtcgacgactggatcgaggactacgccagcaggatcgacttcaaggcggaccgcgatcagatcgttaaagcggttctggcaggattgaaataatagaggaactattacgatgagcgtaaacagcaacgcatacgacgccggcatcatgggcctgaaaggcaaggacttcgccgatcagttctttgccgacgaaaaccaagtggtccatgaaagcgacacggtcgttctggtcctcaagaagtcggacgagatcaatacctttatcgaggagatccttctgacggactacaagaagaacgtcaatccgacggtaaacgtggaagaccgcgcgggttactggtggatcaaggccaacggcaagatcgaggtcgattgcgacgagatttccgagctgttggggcggcagttcaacgtctacgacttcctcgtcgacgtttcctccaccatcggccgggcctataccctgggcaacaagttcaccattaccagtgagctgatgggcctggaccgcaagctcgaagactctcacgcttaaggagaatgacatggcgaaactgggtatacacagcaacgacacccgcgacgcctgggtgaacaagatcgcgcagctcaacaccctggaaaaagcggccgagatgctgaagcagttccggatggaccacaccacgccgttccgcaacagctacgaactggacaacgactacctctggatcgaggccaagctcgaagagaaggtcgccgtcctcaaggcagaagccttcaacgaggtggacttccgtcataagaccgctttcggcgaggatgccaagtccgttctggacggcaccgtcgcgaagatgaacgcggccaaggacaagtgggaggcggagaagatccatatcggtttccgccaggcctacaagccgccgatcatgccggtgaactatttcctggacggcgagcgtcagttggggacccggctgatggaactgcgcaacctcaactactacgacacgccgctggaagaactgcgcaaacagcgcggtgtgcgggtggtgcatctgcagtcgccgcactgaagggaggaagtctcgccctggacgcgacggcatcgccgtgaagtccagggggcagggatgccgttccgggccggcaggctggcccggaatctctggttttcagggggcgtgccggtccacggctcccccctccatctttcgtaaggaaatcaccatggtcgaatcggcatttcagccattttcgggcgacgcagacgaatggttcgaggaaccacggccccaggccggtttcttcccttccgcggactggcatctgctcaaacgggacgagacctacgcagcctatgccaaggatctcgatttcatgtggcggtgggtcatcgtccgggaagaaaggatcgtccaggagggttgctcgatcagcctggagtcgtcgatccgcgccgtgacgcacgtactgaattattttggtatgaccgaacaacgcgccccggcagaggaccggaccggcggagttcaacattgaacaggtaagtttatgcagcgagttcacactatcacggcggtgacggaggatggcgaatcgctccgcttcgaatgccgttcggacgaggacgtcatcaccgccgccctgcgccagaacatctttctgatgtcgtcctgccgggagggcggctgtgcgacctgcaaggccttgtgcagcgaaggggactacgacctcaagggctgcagcgttcaggcgctgccgccggaagaggaggaggaagggttggtgttgttgtgccggacctacccgaagaccgacctggaaatcgaactgccctatacccattgccgcatcagttttggtgaggtcggcagtttcgaggcggaggtcgtcggcctcaactgggtttcgagcaacaccgtccagtttcttttgcagaagcggcccgacgagtgcggcaaccgtggcgtgaaattcgaacccggtcagttcatggacctgaccatccccggcaccgatgtctcccgctcctactcgccggcgaaccttcctaatcccgaaggccgcctggagttcctgatccgcgtgttaccggagggacggttttcggactacctgcgcaatgacgcgcgtgtcggacaggtcctctcggtcaaagggccactgggcgtgttcggtctcaaggagcggggcatggcgccgcgctatttcgtggccggcggcaccgggttggcgccggtggtctcgatggtgcggcagatgcaggagtggaccgcgccgaacgagacccgcatctatttcggtgtgaacaccgagccggaattgttctacatcgacgagctcaaatccctggaacgatcgatgcgcaatctcaccgtgaaggcctgtgtctggcacccgagcggggactgggaaggcgagcagggctcgcccatcgatgcgttgcgggaagacctggagtcctccgacgccaacccggacatttatttgtgcggtccgccgggcatgatcgatgccgcctgcgagctggtacgcagccgcggtatccccggcgaacaggtcttcttcgaaaaattcctgccgtccggggcggcctgaaccggggaagtaccgtgaccaccgagcagttcccgccccaattcctgcgtgaaatgatcgagcagctggacgccagcatccaggagctcgcacgcaaggaaaagggacttgcggcatccctgggcacgggccgggtcgccgagctcaaggaatactgggaccacgtgtggacaagcatagcatagccatttcggtgatgttatatcgcgttgattattgatgctgtttttagttttaacggcaattaatatatatgttattaattgaatgaattttattattcattatatatatgtgtagaatcgtgcgcaggagaaatattcactcaggaagttattactcaggaagcaaagaggattacagaattatctcataacaagtgttaagggatgttatttcccagttctctgtggcataataaacgagtagatgctcattctatctcttatgttcgccttagtgcctcataaactccggaatgacgcagagccgtttacggtgcttatcgtccactgacagatgtcgcttatgcctcatcagacaccatggacacaacg